SEQUENCE LISTING (122 sequences)

SEQ ID NO: 1 - protein, 572 aa, synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLTSLPFQNIHPITIGKCPKYVKSTKLRLATGLRNVPSIQSRGLFGAIAGFIEGGWTGMVDGWYGYHHQNEQGSGYAADLKSTQNAIDITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSSVEYSLQCRICI

SEQ ID NO: 2 - protein, 560 aa, synthetic construct
MKVKLLVLLCTFTATYADTICIGYHANNSTDTVDTVLEKNVTVTHSVNLLEDSHNGKLCLLKGIAPLQLGNCSVAGWILGNPECELLISKESWSYIVETPNPENGTCYPGFADYEELREQLSSVSSFERFEIFPKESSWPNHTVTKGVSASCSHNGSSFYRNLLWLTGKNGLYPNLSKSYANNKEKEVLVLWGVHHPPNIGDQLYHTENAYVSVVSSHYSRFTPEIAKRPKVRDQEGRINYYWTLLEPGDTIIFEANGNLIAPRYAFALSRGFGSGIISNAPMDECDAKCQTPQGAINSSLPFQNVHPVTIGECPKYVRSAKLRMVTGLRNIPSIQSRGLFGAIAGFIEGGWTGMVDGWYGYHHQNEQGSGYAADQKSTQNAINGITNKVNSVIEKMNTQFTAVGKEFNKLERRMENLNKKVDDGFLDIWTYNAELLVLLENERTLDFHDSNVKNLYEKVKSQLKNNAKEIGNGCFEFYHKCNDECMESVKNGTYDYPKYSEESKLNREKIDGVKLESMGVYQILAIYSTVASSLVLLVSLGAISFWMCSNGSLQCRICI

SEQ ID NO: 3 - protein, 565 aa, synthetic construct
MKVKLLVLLCTFTATYADTICIGYHANNSTDTVDTVLEKNVTVTHSVNLLENSHNGKLCLLKGIAPLQLGNCSVAGWILGNPECELLISKESWSYIVEKPNPENGTCYPGHFADYEELREQLSSVSSLERFEIFPKESSWPNHTVTGVSASCSHNGESSFYRNLLWLTGKNGLYPNLSKSYANNKEKEVLVLWGVHHPPNIGDQRALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSVQSRGLFGAIAGFIEGGWTGMIDGWYGYHHQNEQGSGYAADQKSTQNAINGITNKVNSVIEKMNIQFTAVGKEFNKLERRMENLNKKVDDGFIDIWTYNAELLVLLENERTLDFHDSNVKNLYEKVKSQLKNNAKEVGNGCFEFYHKCNDECMESVKNETYDYPKYSEESKLNREKIDGVKLDSMGVYQILTIYSTVASSLVLLVSLGAISFWMCSNGSLQCKICI

SEQ ID NO: 4 - protein, 566 aa, synthetic construct
MKAILVVLLYTFTTANADTLCIGYHANNSTDTVDTILEKNVTVTHSVNLLEDKHNGKLCKLRGVAPLHLGKCNIAGWILGNPECESLSTASSWSYIVETSNSDNGTCYPGDFINYEELREQLSSVSSFERFEIFPKTSSWPNHDPNKGVTTACPHAGAKSFYKNLIWLVEKGNSYPKLSKSYINDKGKEILVLWGIHHPSTSADQQSLYQNADTYVFVGTSKYSKKFKPEIAIRPKVRDQEGRMNYYWTLIEPGDKITFETTGNLVVPRYAFAMERNAGSGIIISDTSVHDCNTTCQTPKGAINTSLPFHNIHPITIGECPKYVRSTKLRMVTGLRNIPSIQSRGLFGAIAGFIEGGWTGMVDGWYGYHHQNAQGSGYAADQKSTQNAIDEITNKVNSVIEKMNTQFTAVGKEFNKLEKRMENLNKKVDDGFLDIWTYNAELLILLENERTLDYHDSNVKNLYEKVRSQLKNNAKETGNGCFEFYHKCDNTCMESVKNGTYNYPKYSEEAKLNREKIDGVKLESMGVYQILAIYSTVASSLVLVVSLGAVSFWMCSNGSLRCRICI

SEQ ID NO: 5 - protein, 566 aa, synthetic construct
MEAILVVLLYTFATANADTLCVGYHANNSTDTVDTLLEKNVTVTHSVNLLEDSHNGKLCRLKGIAPLQLGKCSIAGWILGNPECETLSTASSWSYIVETSSSDNGTCYPGDFIDYEELREQLSSVSSFERFEMFPKTSSWPNHDSNKGVTAACPHAGAKGFYKNLIWLVKKGNSYPKLSQSYINDKGKEVLVLWGIHHPSTTADQQSLYQNADAYVFVGTSRYSKKFKPEIATRPKVRDQERRMNYYWTLVEPGDKITFEATGNLVVPRYAFTMERNAGSGIIISDTPVHDCNTTCQTPEGAINTSLPFQNIHPITIGKCPKYVKSTKLRLATGLRNVPSIQSRGLLGAIAGFIEGGWTGMMDGWYGYHHQNGQGSGYAADLKSTQNAIDKITNKVNSVIDKMNTQFTAVGKEFNHLEKRIENLNKKVDDGFIDVWTYNAELLVVLENERTLEYHDSNVKNLYEKVRNQLKNNAKEIGNGCFEFYHKCNNECMESVKNGTYDYPKYSEEAKLNREEIDGVKLESTRIYQILAIYSTAASSLVLVVSLGAISFWMCSNGSLQCRICI

SEQ ID NO: 6 - protein, 566 aa, synthetic construct
MKAILVVLLYTFATANADTLCIGYHANNSTDTVDTVLEKNVTVTHSVNLLEDKHNGKLCKLRGVAPLHLGKCNIAGWILGNPECESLSTASSWSYIVETSSSDNGTCYPGDFIDYEELREQLSSVSSFERFEIFPKTSSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFIEGGWTGMVDGWYGYHHQNEQGSGYAADLKSTQNAIDKITNKVNSVIEKMNTQFTAVGKEFNHLEKRIENLNKKVDDGFLDIWTYNAELLVLLENERTLDYHDSNVKNLYEKVRSQLKNNAKEIGNGCFEFYHKCDNTCMESVKNGTYDYPKYSEEAKLNREEIDGVKLESTRIYQILAIYSTVASSLVLVVSLGAISFWMCSNGSLQCRICI

SEQ ID NO: 7 - protein, 563 aa, synthetic construct
MKVKLLVLLCTFTATYADTICIGYHANNSTDTVDTILEKNVTVTHSVNLLENSHNGKLCLLKGIAPLQLGNCSVAGWILGNPECELLISKESWSYIVEKPNPENGTCYPGDFINYEELREQLSSVSSLERFEIFPKESSWPNHTVTGVSASCSHNGESSFYRNLLWLTGKNGLYPNLSKSYANNKEKEVLVLWGVHHPPNIGDQRALYHTENAYVSVVSSHYSRKFTPEIAKRPKVRDQEGRINYYWTLLEPGDTIIFEANGNLIAPRYAFALSRGFGSGIINSNAPMDKCDAKCQTPQGAINSSLPFQNVHPVTIGECPKYVRSAKLRMVTGLRNIPSIQSRGLLGAIAGFIEGGWTGMIDGWYGYHHQNAQGSGYAADQKSTQNAIDEITNKVNSVIEKMNIQFTAVGKEFNKLERRMENLNKKVDDGFIDIWTYNAELLILLENERTLDFHDSNVKNLYEKVRNQLKNNAKEVGNGCFEFYHKCNDECMESVKNGIYDYPKYSEESKLNREKIDGVKLESMGVYQILAIYSTVASSLVLLVSLGAISFWMCSGHSCRICI

SEQ ID NO: 8 - protein, 566 aa, synthetic construct
MEAILVVLLYTFTTANADTLCVGYHANNSTDTVDTVLERNVTVTHSVNLLEDSHNGKLCRLKGIAPLQLGKCNIAGWLLGNPECESLFSKESWSYIVETPNPENGTCYPGHFADYEELREQLNSVSSFERFEMFPKTSSWPDHDSNKGVTTACPHAGAKGFYKNLIWLTKKGNSYPKLSQSYINDKGKEILVLWGIHHPSTTADQQSLYQNADTYVFVGTSKYSKKFKPEIATRPKVRDQERRMNYYWTLIEPGDKITFEANGNLVVPRYAFTMERNAGSGIIISDTSVHDCNTTCQTPEGAINTSLPFHNIHPITIGECPKYVRSTKLRMVTGLRNVPSVQSRGLFGAIAGFIEGGWTGMMDGWYGYHHQNGQGSGYAADQKSTQNAINGITNKVNSVIDKMNTQFTAVGKEFNKLEKRMENLNKKVDDGFIDVWTYNAELLILLENERTLDYHDSKVKNLYEKVKSQLKNNAKETGNGCFEFYHKCNNECMESVKNGTYDYPRYSEEAKLNREKIDGVKLESTRVYQILAIYSTAASSLVLVVSLGEISFWMCSNGSLRCRICI

SEQ ID NO: 9 - protein, 558 aa, synthetic construct
MAIIYLILLFTAVRGDQICIGYHANNSTEKVDTILERNVTVTHAKDILEKTHNGKLCKLNGIPPLELGDCSIAGWLLGNPECDRLLVPEWSYIMEKENPRGLCYPGSFNDYEELKHLLSSVKHFEKVKILPKDRWTQHTTTGGSRACAVSGNPSFFRNMVWLTKKGSNYPVAKGSYNNTSGEQMLIIWGVHHPNDETEQRTLYQNVGTYVSVTSTLNKRSTPEIARPKVNGLGSRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIESRGLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEIKGVKLSSMGVYQILAIYATVAGSLSLAIMMAGISFWMCSNGSLQCRICI

SEQ ID NO: 10 - protein, 562 aa, synthetic construct
MVIIYLILLFTAVRGDKICIGYHANNSTEMVDTILERNVTVTHAKDILEKTHNGKLCKLNGIPPLELGNCSIAGWLLGNPECDRLLRVPEWSYIMEKENPRDGLCYPGSFNDYEELKHFLSSVKHFEKVKILPKDRWTQHTTTGGSRACAVSGNPSFFRNMVWLTKKGSNYPVAQGSYNNTSGEKMLIIWGVHHPNDETEQRTLYQNVGTYVSVGTSTLNKRSTPDIATRPKVNGLGGRMEFSWTLLDMWDTINFESTGNLIAPEYGFKISKRGRSGIMKTEGTLENCETKCQTPLGAINTTLPFHNVHPLAIGECPKYVRSEKLVLATGLRNVPQIESRGLFGAIAGFIEGGWQGMIDGWYGYHHSNDQGSGYAADKESTQRAFDGITNKVNSVIEKMNTQFESVGKEFSNLERRLENLNKKMEDGFLDVWTYNAELLVLMENERTLGFHDSNVKNLYNKVRMQLRDNVKELGDGCFEFYHKCNDECMNSVKNGTYYYPKYEEESKLNRNEIKGVKLSSMGIYQILAIYATVAGSLSLAIMMAGISFWMCSNGSLQFRICI

SEQ ID NO: 11 - protein, 562 aa, synthetic construct
MAIIYLILLFATVRGDQICIGYHANNSTETVDTILERNVTVTHAKDILEKAHNGKLCKLNGIPPLELGDCSIAGWLLGNPKCDRLLSVPEWSYIMEKENPKYSLCYPGSLNDYEELKHLLSSVKHFEKVRILPKDGWTQHTTTGGSKACAVSGKPSFFRNMVWLTEKGSNYPVAKGSYNNTSGEQMLIIWGVHHPIDEAEQRALYQEVGTYVSASTSTLNKRSIPEIAARPKVNGLGSRMEFFWTLLDVWDTINFESTGNLVVPEYGFKISKRGSSGIMKTEGTLGNCETKCQTPLGAINTTLPFHNVHPLTIGECPRYVKSEKLVLATGLRNIPQIESRGLFGAIAGFIEGGWQGMVDGWYGYHHSNEQGSGYAADKESTQKAFDGITNKENSVIEKMNTQFEAVGKEFNNLEKRLENLNKRMEDGFLDVWTYNAELLVLMENERTLDFHDSNVKNLYEKVRMQLRDNVKELGNGCFEFYHKCDNECMDSVKNGTYDYPKYEEESKAKRNEIKGVKLSSMGVYQILAIYATVAGSLSLAIMVAGISFWMCSNGSLQCRICI

SEQ ID NO: 12 - protein, 562 aa, synthetic construct
MAIIYFILLFTAVRGDQICIGYHANNSTEKVDTILERNVTVTHAKNILEKTHNGRLCKLNGIPPLELGDCSITGWLLGNPECDRLLSVPEWSYIMEKENPRYSLCYPGSFNDYEELKHLLSSVKHLEKVKILPKDGWTQHETDGGSKACAVSGEPSFFRNMVWLTKKGPNYPVAKRSYNNTSGEQMLIIWGVHHPNDEAEQRALYQKVGTYVSASTSTLNKRSTPEIAARPEVNGLGSRMEFSWTLLDVWDTISFESTGNLVAPEYGFKISKRGSSGIIKTEGTLGNCETKCQTPLGAINTTLPFHNVHPLTIGECPKYVKSEKLVLATGPRNVPQIESRGLFGAIAGFIEGGWQGMVDGWYGYHHSKEQGSGYAADKESTQKAFDRITNKVNSVIEKINTQFEAVGKEFSNLEKRLENLNKKMEDGFIDVWTYNAELLVLMENERILDFHDSNVKNLYDKVRMQLRDNAKELGNGCFEFYHKCDDECMNSVKNGTYDYPKYEEESKLKRNEIKGVELSSMGVYQILVIYATVAGSLSLAIMIAGISFWMCSNGSLQCRVCI

SEQ ID NO: 13 - protein, 562 aa, synthetic construct
MAIIYLILLFTAVRGDQICIGYHANNSTEKVDTILERNVTVTHAKDILEKTHNGKLCKLNGIPPLELGDCSIAGWLLGNPECDRLLSVPEWSYIMEKENPRDGLCYPGSFNDYEELKHLLSSVKHFEKVKILPKDRWTQHTTTGGSRACAVSGNPSFFRNMVWLTKKGSNYPVAKGSYNNTSGEQMLIIWGVHHPNDETEQRTLYQNVGTYVSVGTSTLNKRSTPEIATRPKVNGLGSRMEFSWTLLDMWDTINFESTGNLIAPEYGFKISKRGSSGIMKTEGTLENCETKCQTPLGAINTTLPFHNVHPLTIGECPKYVKSEKLVLATGLRNVPQIESRGLFGAIAGFIEGGWQGMVDGWYGYHHSNDQGSGYAADKESTQKAFDGITNKVNSVIEKMNTQFEAVGKEFSNLERRLENLNKKMEDGFLDVWTYNAELLVLMENERTLDFHDSNVKNLYDKVRMQLRDNVKELGNGCFEFYHKCDDECMNSVKNGTYDYPKYEEESKLNRNEIKGVKLSSMGVYQILAIYATVAGSLSLAIMMAGISFWMCSNGSLQCRICI

SEQ ID NO: 14 - protein, 562 aa, synthetic construct
MAIIYLILLFATVRGDQICIGYHANNSTETVDTILERNVTVTHAKNILEKTHNGKLCKLNGIPPLELGDCSIAGWLLGNPECDRLLRVPEWSYIMEKENPRYSLCYPGSLNDYEELKHLLSSVKHFEKVRILPKDGWTQHTTTGGSKACAVSGKPSFFRNMVWLTKKGPNYPVAKRSYNNTSGEQMLIIWGVHHPNDEAEQRALYQKVGTYVSASTSTLNKRSTPDIATRPKVNGQGGRMEFSWTLLDVWDTINFESTGNLVAPEYGFKISKRGSSGIMKTEGTLGNCETKCQTPLGAINTTLPFHNVHPLTIGECPKYVKSEKLVLATGPRNVPQIESRGLFGAIAGFIEGGWQGMVDGWYGYHHSNDQGSGYAADKESTQKAFDRITNKVNSVIEKMNTQFEAVGKEFNNLEKRLENLNKKMEDGFLDVWTYNAELLVLMENERTLDFHDSNVKNLYEKVRMQLRDNVKELGNGCFEFYHKCDNECMDSVKNGTYDYPKYEEESKLKRNEIKGVKLSSMGIYQILAIYATVAGSLSLAIMIAGISFWMCSNGSLQCRICI

SEQ ID NO: 15 - protein, 562 aa, synthetic construct
MAIIYFILLFTAVRGDQICIGYHANNSTEKVDTILERNVTVTHAKNILEKTHNGKLCKLNGIPPLELGDCSIAGWLLGNPECDRLLSVPEWSYIMEKENPKYSLCYPGSFNDYEELKHLLSSVKHFEKVKILPKDGWTQHETDGGSKACAVSGEPSFFRNMVWLTEKGSNYPVAKGSYNNTSGEQMLIIWGVHHPIDEAEQRALYQEVGTYVSASTSTLNKRSIPEIAARPKVNGLGGRMEFSWTLLDVWDTISFESTGNLVAPEYGFKISKRGSSGIMKTEGTLENCETKCQTPLGAINTTLPFHNVHPLTIGECPKYVKSEKLVLATGPRNVPQIESRGLFGAIAGFIEGGWQGMVDGWYGYHHSNDQGSGYAADKESTQRAFDGITNKVNSVIEKMNTQFEAVGKEFSNLEKRLENLNKKMEDGFLDVWTYNAELLVLMENERTLDFHDSNVKNLYNKVRMQLRDNVKELGNGCFEFYHKCDDECMSSVKNGTYDYPKYEEESKLKRNEIKGVKLSSMGVYQILAIYATVAGSLSLAIMVAGISFWMCSNGSLQCRICI

SEQ ID NO: 16 - protein, 564 aa, synthetic construct
MKTIIALSYILCLVFAQKLPGNDNNSTATLCLGHHAVPNGTIVKTITNDQIEVTNATELVQSSTGEICDSPHQILDGENCTLIDALLGDPQCDGFQNKKWDLFVERSKAYSNCYPYDVPDYASLRSLVASSGTLEFNNESFNWTGVTQNGTSSACIRRSNSFFSRLNWLTHLNFKYPALNVTMPNNEQFDKLYIWGVHHPGTDKDQIFLYAQSGRITVSTKRSQQAVIPNIGSRPRRIPSRISIYWTIVKPGDILLINSTGNLIAPRGYFKIRSGKSSIMRSDAPIGKCSECITPNGSIPNDKPFQNVNRITYGACPRYVKQTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNIRCNICI

SEQ ID NO: 17 - protein, 566 aa, synthetic construct
MKTIIALSHILCLVFAQKIPGNDNSTATLCIGHHAVPNGMIVKTITNDRIEVTNATELVQNSSIGEICDSPHQILDGENCTLIDALLGDPHCDGFQNEKWDLFVERSKAFSNCYPYDVPDYASLRSLVASSGTLEFKNESFNWTGVAQNGTSSACIRRSSSSFFSRLNWLTHLNYTYPALNVTMPNKEQFDKLYIWGVHHPGTDKDQIFLYAQSSGRITVSTKRSQQTAIPNIGSRPRIRDIPSRISIYWTIVKPGDVLVINSNGNLIAPRGYFKIQSGKSSIMRSDAPIGKCKSECITPNGSISNDKPFQNVNKITYGACPRYVKHSTLKLATGMRNVPEKQTRGLFGAIAGFIENGWEGMKDGWYGFRHQNSEGRGQAADLKSTQAAINQINGKLNRLIGKTNEKFHQIEKEFSEVEGRVQDLEKYVEDSKIDLWSYNAGLLVALENQHTIDLTDSEMNKLFERTRKQLRENAEDMGNGCFTIYHKCDNACIGSIRNGTYDHNVYRDEALNKRFQIKGIELKSGYKDWILWISFAISCFLLCVALLGFIMWACQKGNIRCNICI

SEQ ID NO: 18 - protein, 566 aa, synthetic construct
MKTIIALSYIFCLVFAQKLPGNDNSTAMLCLGHHAVPNGTLVKTITNDQIEVTNATELVQSSSTGRICDSPHRILDGKNCTLMDALLGDPHCDGFQNKEWDLFVERSKAYSNCYPYDVPDYVSLRSLVASSGTLEFNNESFNWAGVTQNGTSSACKRRSNSSFFSRLNWLTHLNFKYPALNVTMPNNEQFDKLYIWGVHHPSTDSDQISLYAQASGRVTVSTKRSQQAVIPNIGSRPRIRNIPSRISIYWTIVKPGDILVINSNGNLIAPRGYFKIRNGKSSIMRSNAPIGKCNSECITPNGSIPNDKPFQNVNRVTYGACPRYVKQSTLKLATGMRNVPEKQTRGIFGAIAVFIENGWEGMVDGWYGFRHQNSEGTGQAADLKSTQAAVDQINGKLNRLIEKTNEKFHQIEKEFTEVEGRIQDLEKYVEDTKVDLWSYNAELLVALENQHTIDLTDAEMNKLFERTKKQLRENAEDIGNGCFKIYHKCDNACIESIRNGTYDHDVYRDEALNNRFHIKGVELKSGYKNWILWISFAISCFLLCVVLLGFIMWACQKGNIRCSICI

SEQ ID NO: 19 - protein, 566 aa, synthetic construct
MKTIIALSYILCLVFAQKLPENDNSTATLCLGHHAVPNGTIVKTITNDQTEVTNATELVQSSSTGEICNSPHQILDGKNCTLIDALLGDPQCDGFQNKKWDLFVERSKAHSNCYPYDVPDYASLRSLIASSGTLEFNNESFNWTGVTQNGTSSACIRRSNNSFFSRLNWLTHLKFKYPALNVTMPNNEKFDKLYIWGVHHPGTDNDQIFLYAQASGRITVSTKRSQQTVIPNIGSRPRVRNIPSRVSIYWTIVKPGDILLINSTGNLIAPRGYFKIRSGKSSIMRSDAPIGKCNSACITPNGSIPNGKPFQNVNRITYGACPRYVKQNTLKLATGMRNVPERQTRGIFGAIAGFIENGWEGMMDGWYGFRHQNSEGIGQAADLKSTQAAIDQINGKLNRVIEKTNEKFHQIEKEFSEVEGRIQDIEKYVEDTKIDLWSYNAEFLVALENQHTIHLTDSEMNKLFEKTKKQLRENAEDMGNGCFKIYHKCNNACIGSIRNETYDHNVYRDEALNNRFQIKGVELKSGYKDWVLWISFAISCFLLCIVLLGFIMWACQRGNIRCNICI

SEQ ID NO: 20 - protein, 566 aa, synthetic construct
MKTIIALSYILCLVFAQKLPGNDNSTATLCLGHHAVPNGTIVKTITNDQIEVTNATELVQNSSIGEICDSPHQILDGENCTLIDALLGDPQCDGFQNKKWDLFVERSKAYSNCYPYDVPDYASLRSLVASSGTLEFNNESFNWTGVTQNGTSSACIRRSNNSFFSRLNWLTHLNFKYPALNVTMPNNEQFDKLYIWGVHHPGTDKDQIFLYAQSSGRITVSTKRSQQAVIPNIGSRPRIRDIPSRISIYWTIVKPGDILLINSTGNLIAPRGYFKIRSGKSSIMRSDAPIGKCKSECITPNGSIPNDKPFQNVNRITYGACPRYVKQNTLKLATGMRNVPEKQTRGIFGAIAGFIENGWEGMVDGWYGFRHQNSEGRGQAADLKSTQAAIDQINGKLNRLIGKTNEKFHQIEKEFSEVEGRIQDLEKYVEDTKIDLWSYNAELLVALENQHTIDLTDSEMNKLFEKTKKQLRENAEDMGNGCFKIYHKCDNACIGSIRNGTYDHDVYRDEALNNRFQIKGVELKSGYKDWILWISFAISCFLLCVALLGFIMWACQKGNIRCNICI

SEQ ID NO: 21 - protein, 566 aa, synthetic construct
MKTIIALSHILCLVFAQKIPGNDNSTAMLCLGHHAVSNGTIVKTITNDRIEVTNATELVQSSSTGEICDSPHRILDGKNCTLIDALLGDPHCDGFQNKEWDLFVERSKAHSNCYPYDVPDYVSLRSLVASSGTLEFKNESFNWTGVAQNGTSSACIRRSSSSFFSRLNWLTHLNYTYPALNVTMPNNEKFDKLYIWGVHHPGTDNDQIFLYAQASGRITVSTKRSQQTVIPNIGSRPRVRNIPSRISIYWTIVKPGDVLVINSNGNLIAPRGYFKIQSGKSSIMRSDAPIGKCNSECITPNGSISNDKPFQNVNKITYGACPRYVKHSTLKLATGMRNVPERQTRGIFGAIAVFIENGWEGMMDGWYGFRHQNSEGTGQAADLKSTQAAINQINGKLNRLIEKTNEKFHQIEKEFSEVEGRVQDLEKYVEDSKIDLWSYNAGLLVALENQHTIHLTDSEMNKLFERTKKQLRENAEDMGNGCFTIYHKCDNACIESIRNGTYDHNVYRDEALNKRFQIKGIELKSGYKDWVLWISFAISCFLLCVVLLGFIMWACQRGNIRCNICI

SEQ ID NO: 22 - protein, 566 aa, synthetic construct
MRTIIALSYIFCLVFAQKLPENDNSTATLCIGHHAVPNGTLVKTITNDQVEVTNATELVQSSSTGGICDSPHQILDGKNCTLVDALLGDPHCDGFQNEKWDLFVERSKAFSNCYPYDVQDYASLRSLIASSGTLEFNNESFNWAGVTQNGTSSACKRRSNSSFFSRLNWLTHLKFKYPALNVTMPNKEQFDKLYIWGVHHPSTDSDQISLYAQASGRVTVSTKRSQQTAIPNIGSRPRIRNIPSRVSIYWTIVKPGDILLINCTGNLIAPRGYFKIRNGKSSIMRSNAPIGKCNSACITPNGSIPNGKPFQNVNRVTYGACPRYVKQSTLKLATGMRNVPEKQTRGLFGAIAGFIENGWEGMKDGWYGFRHQNSEGIGQAADLKSTQAAVDQINGKLNRVIEKTNEKFHQIEKEFTEVEGRIQDIEKYVEDTKVDLWSYNAELLVSLENQHTIDMTDSEMNKLFERTRKQLRENAEDIGNGCFKIYHKCNNACIGSIRNETYDHNVYRDEALNNRFHIKGVELKSGYKNWILWISFATSCFLLCIVLLGFIMWACQKGNIKCNICI

SEQ ID NO: 23 - protein, 573 aa, synthetic construct
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGSSFFRNVVWLIKKNNTYPTIKRSYNNTNQEDLLVLWGIHHPNDAAEQTLYQNPTTYISVGTSTLNQRLVPKIATRSKVNGQSGRMEFFWTILKPNDAINFESNGNFIAPEYAYKIVKKGDSTIMKSELEYGNCNTKCQTPIGAINSSMPFHNIHPLTIGECPKYVKSNRLVLATGLRNSPQRERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICIKFCEFRL

SEQ ID NO: 24 - protein, 566 aa, synthetic construct
MEKIVLLLAIVSIVKSDQICIGYHANNSSEQVDTIMERNVTVTHAQDILEKTHNGKLCNLDGVKPLILRDCSVAGWLLGNPMCDEFLNVPEWSYIVEKINPANDLCYPGNFNDYEELKHLLSRITHFEKIQIIPKNSWSDHEASGVSSACPYQGRSSFFRNVVWLTKKDNAYPTIKRSYNNTNQEDLLVLWGIHHPNDAAEQTRLYQNPTTYISVGTSTLNQRLVPRIATRSKINGQSGRIEFFWTILKSNDAINFESNGNFIAPENAYKIVKKGDSTIMKSELEYGNCNTKCQTPIGAINSSMPLHNIHPLTIGECPKYVKSNRLVLATGLRNSPQGERRRKKRGLFGAIACFIEGGWQGMVDGWYGSHHSNDQGSGYAADKESTQRAIDGVTNKVNSIINKMNTQFEAVGREFNNLERRIGNLNKKMEDGFLDVWTYNAELLVLMGNERTLDFHDSNVKNLYDKVRLQLKDNAKELGNGCFEFYHRCDNECMESVRNGTYDYPQYSEEARLRREEISGVKLESIGVYQILSIYSTAASSLALAIMVAGLFLWMCSNGSLHADCI

SEQ ID NO: 25 - protein, 568 aa, synthetic construct
MEKIVLLFAIVSLVKSDQICIGYHANNSTKQVDTIMEKNVTVTHAQDILEKKHNGKLCDLDGVRPLILRDCSVAGWLLGNPMCDEFINVPEWSYIVEKANPVNDLCYPGDFNDYEELKHLLSRINHFEKIQIIPKSSWSSHEASLGVSSACPYQGKSSFFRNVVWLIKKNNTYPTIKRSYNNTNQEDLLVMWGIHHPNDEAEQIKLYQNPTTYVSVGTSTLNQRLTPRIATRSKVNGQSGRMEFFWTILKPNDAINFESNGNFVAPEYAYKIVKKGDSAIMKSELEYSNCNTKCQTPMGAINSSMPFHHIHPLTIGECPKYVKSTRLVLATGLRNSPQRESRRKKRGLFGAIAGFIEAGWQGMVDGWYGYHHSNEQGSGYAADKESTQKAIDGVTNKVNSIIDKMNTQFEAIGREFNNLERRIENLNKKMEDGFLDVWTYKAELLVLMENERTLDYHDSNVRNLYDKVRLQLRDNTKELGNGCFEFYHKCDDECMESVKNGTYDYPQYSEEARLNREEISGVKLESIGIYQILSIYSTVASSLVLAIMVAGLSLWMCSNGSLQCRVCI

SEQ ID NO: 26 - protein, 574 aa, synthetic construct
MEQIVLLLAIVSLVKSDHICIGYHANNSTEQVDTIMEKNVTVTHAQNILEKTHNGKLCDLDGVKPLILKDCSVAGWLLGNPMCDEFIRVPEWSYIVEKANPANDLCYPGSFNDYEELKHLLSRINHFEKLQIIPKSSWSDHEASSGVSSACPYQGTPSFFRNVVWLIKKNSTYPTIKKSYNNTNQEDLLILWGIHHSNDAAEQTKLYQNPTTYISIGTSTLNQRLVPKIATRSKVNGQSGRMDFFWTILKPNDAIHFESNGNFIAPEYAYKIVKEGDSAIMKSEVEYGNCNTKCQTPVGAINSSMPFHNIHPLTIGECPKYVKSNKLVLATGLRNSPLRERRRKRGLFGAIAGFIEGGWQGMVDGWYGFHHSNEQGSGYAADRESTQKAIDGVTNKVNSIIDKMNTQFEVVGREFNNLESRIENLNKKMEDGFLDVWTYNAELLVLMENERTLDFHDSNVKNLYDRVRLQLRDNAKELGNGCFEFYHKCDNECMESIRNGTYNYPQYSEEARLKREEISGVKLESIGTYQILSIYSTVASSLALAIMIAGLSLWMCSNGSLQCRICIYFCEFRL

SEQ ID NO: 27 - protein, 569 aa, synthetic construct
MEKIVLLLAIVSLVKSDQICIGYHANNSTEQVDTIMEKNVTVTHAQDILEKTHNGKLCDLDGVKPLILRDCSVAGWLLGNPMCDEFINVPEWSYIVEKANPANDLCYPGNFNDYEELKHLLSRINHFEKIQIIPKSSWSDHEASSGVSSACPYQGRSSFFRNVVWLIKKNNTYPTIKRSYNNTNQEDLLVLWGIHHPNDAAEQTRLYQNPTTYISVGTSTLNQRLVPKIATRSKVNGQSGRMEFFWTILKPNDAINFESNGNFIAPEYAYKIVKKGDSTIMKSELEYGNCNTKCQTPIGAINSSMPFHNIHPLTIGECPKYVKSNRLVLATGLRNSPQGERRRKKRGLFGAIAGFIEGGWQGMVDGWYGYHHSNEQGSGYAADKESTQKAIDGVTNKVNSIIDKMNTQFEAVGREFNNLERRIENLNKKMEDGFLDVWTYNAELLVLMENERTLDFHDSNVKNLYDKVRLQLRDNAKELGNGCFEFYHKCDNECMESVRNGTYDYPQYSEEARLKREEISGVKLESIGTYQILSIYSTVASSLALAIMVAGLSLWMCSNGSLQCRICIK

SEQ ID NO: 28 - protein, 566 aa, synthetic construct
MEKIVLLFAIVSLVKSDQICIGYHANNSTKQVDTIMEKNVTVTHAQDILEKTHNGKLCNLDGVKPLILKDCSVAGWLLGNPMCDEFLNVPEWSYIVEKINPANDLCYPGDFNDYEELKHLLSRINHFEKIQIIPKNSWSDHEASGVSSACPYQGTPSFFRNVVWLIKKNSTYPTIKKSYNNTNQEDLLILWGIHHSNDAAEQTKLYQNPTTYISIGTSTLNQRLTPRIATRSKVNGQSGRMDFFWTILKSNDAINFESNGNFIAPENAYKIVKKGDSAIMKSEVEYGNCNTKCQTPMGAINSSMPFHNIHPLTIGECPKYVKSNKLVLATGLRNSPLRERRRKRGLFGAIAGFIEGGWQGMVDGWYGFHHSNEQGSGYAADKESTQRAIDGVTNKVNSIINKMNTQFEAVGREFNNLERRIENLNKKMEDGFLDVWTYNAELLVLMENERTLDFHDSNVKNLYDKVRLQLKDNAKELGNGCFEFYHRCDNECMESIRNGTYNYPQYSEEARLNREEISGVKLESIGIYQILSIYSTAASSLALAIMVAGLFLWMCSNGSLQCRVCI

SEQ ID NO: 29 - protein, 568 aa, synthetic construct
MEKIVLLLAIVSIVKSDQICIGYHANNSTKQVDTIMEKNVTVTHAQDILEKKHNGKLCDLDGVRPLILRDCSVAGWLLGNPMCDEFIRVPEWSYIVEKANPVNDLCYPGVFNDYEELKHLLSRINHFEKLQIIPKSSWSSHEASLGVSSACPYQGKSSFFRNVVWLTKKDNAYPTIKRSYNNTNQEDLLVMWGIHHPNDAAEQTKLYQNPTTYVSVGTSTLNQRLVPRIATRSKVNGQNGRMEFFWTILKSNDVINFESNGNFIAPEYAYKIVKEGDSAIMKSELEYSNCNTKCQTPMGAINSSMPFHNIHPLTIGECPKYVKSTRLVLATGLRNSPQRESRRKKRGLFGAIAGFIEGGWQGMVDGWYGFHHSNEQGSGYAADRESTQKAIDGVTNKVNSIINKMNTQFEAVGREFNNLERRIENLNKKMEDGFLDVWTYNAELLVLMENERTLDFHDSNVKNLYDKVRLQLRDNAKELGNGCFEFYHKCDNECMESVKNGTYDYPQYSEEARLNREEISGVKLESIGVYQILSIYSTVASSLALAIMIAGLSLWMCSNGSLQCRICI

SEQ ID NO: 30 - protein, 472 aa, synthetic construct
MNPNQKIITIGSVCMTIGMANLILQIGNIISIWSHSIQLGNQNQIETCTICNQSVITYENNTWVNQTYVNISNTNFAAGQSVVSVKLAGNSSLCPVSGWAIYSKDNSIRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTIMTDGPSDGQASYKIFRIEKGKIVKSVEMNAPNYHYEECSCYPDSSEITCVCRDNWHGSNRPWVSFNQNLEYQIGYICSGVFGDNPRPNDKTGSCGPVSSNGANGVKGFSFKYGNGVWIGRTKSISSRKGFEMIWDPNGWTGTDNFSIKQDIVGINEWSGYSGSFVQHPELTGLDCIRPCFWVELIRGRPKENTTIWTSGSSISFCGVNSDTVGWSWPDGAELPFTIDKY

SEQ ID NO: 31 - protein, 470 aa, synthetic construct
MNPNQKIITIGSISIAIGIISLMLQIGNIISIWASHSIQTGSQNHTGICNQRIITYENSTWVNHTYVSINNTNVVAGKDKTSVTLAGNSSLCSISGWAIYTKDNSIRIGSKGDVFVIREPFISCSHLECKTFFLTQGALLNDKHSNGTVKDRSPYRALMSCPLGEAPSPYNSRFESIAWSASACHDGINWLTIGISGPDDGAVAVLKYNGIITGTIKSWKKQILRTQESECVCVNGSCFTVMTDGPSNGQASYKIFRIEKGKIVKSVEMNAPNYHYEECSCYPDSSEITCVCRDNWHASNRPWVSFNQNLDYQIGYICSGVFGDNPRPEDGEGSCNPVTVDGANGVKGFSYKYDNGVWIGRTKSNRLRKGFEMIWDPNGWTNTDSDFSVKQDVVAITDWSGYSGSFVQYPELTGLDCMRPCFWVELVRGLPRENTTIWTSGSSISFCGVNSDTANWSWPDGAELPFTIDK

SEQ ID NO: 32 - protein, 469 aa, synthetic construct
MNPTQKIITIGSVCMTIGMANLILQIGNIISIWISHSIQLGNQNQIETCNQSIITYENNTWVNQTYVNISNTNFAAGQSVVSVKLAGNSSLCPVSGWAIYSKDNSIRIGSRGDVFVIRESFISCSHLECRTFFLTQGALLNDRHSNGTVKDRSPHRTLMSCPVGEAPSPYNSKFESVAWSASACHDGMGWLTIGISGPDNGAVAVLKYNGIITETIKSWKKRILRTQESECVCMNGSCFTIMTDGPSNGAASYKIFKIEKGKVTKSIELNAPNFHYEECSCYPDTGTVMCVCRDNWHGANRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIVGIKEWSGYSGSFIQHPELTGLNCIRPCFWVELIRGRPKENTIWTSGSSISFCGVDSDTANWSWPDGAELPFIIDK

SEQ ID NO: 33 - protein, 469 aa, synthetic construct
MNPNKKIITIGSICMTIGMANLMLQIGNIISIWVSHSIQIGNQSQIETCNQSVITYENNTWVNHTYVNINNTNVVAGEDKTSVTLAGNSSLCPIRGWAIYSKDNSVRIGSKGDVFVMREPFISCSPLECRTFFLTQGTLLNDKHSNGTIKDRSPYRTLMSCPIGEVPSPYNSRFESVAWSASACHDGTSWLTIGISGPDSGAVAVLKYNGIITDTIKSWRNNILRTQESECACVNGSCFTIMTDGPSDGQASYKIFRIEKGKIIKSVEMKAPNYHYEECSCYPDTGIVMCVCRDNWHGSNRPWVSFNQNLEYQMGYICSGVFGDNPRPKDGKGSCNPVTVDGADGVKGFSYKYGNGVWIGRTKSISSRKGFEMIWDTNGWTGTDNKFSIKQDIVGINEWSGYSGSFVQHPELTGLDCIRPCFWVELIRGRPEENTIWTSGSIISFCGVNSDTVGWSWPDGAELPFTVDK

SEQ ID NO: 34 - protein, 469 aa, synthetic construct
MNPNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSIRIGSKGDVFVIREPFISCSPLECRTFFLTQGALLNDKHSNGTIKDRSPYRTLMSCPIGEVPSPYNSRFESVAWSASACHDGINWLTIGISGPDNGAVAVLKYNGIITDTIKSWRNNILRTQESECACVNGSCFTIMTDGPSDGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRKGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 35 - protein, 470 aa, synthetic construct
MNPTQKIITIGSISIAIGIISLMLQIGNIISIWASHSIQTGSQNHTGICNQRIITYENSTWVNHTYVNINNTNVVAGKDKTSVTLAGNSSLCPIRGWAIYSKDNSVRIGSKGDVFVMREPFISCSHLECRTFFLTQGTLLNDKHSNGTVKDRSPYRALMSCPLGEAPSPYNSKFESVAWSASACHDGMGWLTIGISGPDSGAVAVLKYNGIITGTIKSWKKQILRTQESECVCVNGSCFTVMTDGPSNGAASYKIFKIEKGKVTKSIELNAPNFHYEECSCYPDTGTVMCVCRDNWHGANRPWVSFNQNLDYQIGYICSGVFGDNPRPEDGEGSCNPVTVDGANGVKGFSYKYGNGVWIGRTKSISSRNGFEMIWDPNGWTNTDSDFSVKQDVVAITDWSGYSGSFIQHPELTGLDCMRPCFWVELVRGLPRENTTIWTSGSSMSFCGVNSDTANWSWPDGAELPFIIDK

SEQ ID NO: 36 - protein, 469 aa, synthetic construct
MNPNKKIITIGSICMTIGMANLMLQIGNIISIWVSHSIQIGNQSQIETCNQSIITYENNTWVNHTYVSINNTNVVAGEDKTSVTLAGNSSLCSISGWAIYTKDNSIRIGSRGDVFVIREPFISCSHLECKTFFLTQGALLNDRHSNGTVKDRSPHRTLMSCPVGEAPSPYNSRFESVAWSASACHDGTSWLTIGISGPDDGAVAVLKYNGIITETIKSWKKRILRTQESECVCMNGSCFTIMTDGPSNGQASYKIFRIEKGKIIKSVEMKAPNYHYEECSCYPDTGIVMCVCRDNWHGSNRPWISFNQNLEYQMGYICSGVFGDNPRPKDGKGSCNPVTVDGADGVKGFSYKYDNGVWIGRTKSNRLRKGFEMIWDSNGWTGTDNKFSIKQDIVGIKEWSGYSGSFIQHPELTGLNCIRPCFWVELIRGRPEENTIWTSGSSISFCGVDSDTANWSWPDGAELPFTVDK

SEQ ID NO: 37 - protein, 469 aa, synthetic construct
MNPNQKIITIGSVSLTISTICFFMQIAILITTVTLHFKQYEFNSPPNNQVMLCEPTTIIERNITEIVYLTNTTIEKEICPKPAEYRNWSKPQCITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKYILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 38 - protein, 469 aa, synthetic construct
MNPNQKIIAIGSVSLTIATICFLMQIAILVTTVTLHFKQYECNSPPNNQVMPCEPIIIERNITEIVYLNNTTIEKEICPKLAEYRNWSKPQCNITGFAPFSKDNSIRLSASGDIWVTREPYVSCDPDKCYQFALGQGTTLNNGHSNDTVHDRIPHRTLLMNELGVPFHLGTRQVCIAWSSSSCHDGKAWLHVCVTGHDENATASFIYDGRLVDSIGSWSKKILRTQESECVCINGTCTVVMTDGSASGRADTKILFIEEGKIVHISPLSGSAQHVEECSCYPRYSGVRCVCRDNWKGSNRPIVDINVKDYSIVSSYVCSGLVGDTPRKNDRSSSSYCRNPNNEKGSHGVKGWAFDNGDDVWMGRTISEKLRSGYETFKVIEGWSKPNSKLQINRQVIVERGNRSGYSGIFSVEGKNCINRCFYVELIRGREQETRVWWTSNSIIVFCGTSGTYGTGSWPDGADINLMPI

SEQ ID NO: 39 - protein, 469 aa, synthetic construct
MNPNKKIITIGSVSLTISTICFFMQIAILITTVTLHFKQHECNSPPNNQVMLCEPTIIERNMTEIVYLTNITIEKEICPKPAEYRNWSKPQCGITGFAPFSRDNSIRLSAGGNIWVTREPYVSCDPNKCYQFALGQGTTLNNVHSNNTVRDRTPYRTLLMSELGVPFHLGTKQVCMAWSSSSCHDGKAWLHVCITGDDKNATASIIYNGRLVDSVVSWSKDILRTQESECVCIDGTCTVVMTDGNATGKADTKILFIKEGKIVHTSKLSGSAQHVEECSCYPRYPDVRCICRDNWKGSNRPIVDINIKDHSIVSSYVCSGLVGDTPRKSDSSSSSHCLNPNNEEGGHRVKGWAFDDGDDVWMGRTINETSRLGYETFKVVEGWSNPKSKLQINRQVIVDRGDRSGYSGIFSVESKSCINRCFYVELIRGRKQETEVWWTSNSIVVFCGTSGTYGTGSWPDGADLNLMHI

SEQ ID NO: 40 - protein, 469 aa, synthetic construct
MNPNQKIITIGSVSLTIATVCFLMQIAILATTVTLHFKQYEFNSPPNNQVVLCEPTIIERNITEIVYLTNTTIEKEICPKLVEYRNWSKPQCDITGFAPFSKDNSIRLSAGGDIWVTREPYVSCDPGKCYQFALGQGTTLNNRHSNDTVHDRTPYRTLLMNELGIPFHLGTKQVCIAWSSSSCYDGKAWLHVCVTGDDKNATASFIYNGRLVDSIVSWSKEILRTQESECVCINGTCAVVMTDGSASGKADTKILFIEEGKIVHTSTLSGSAQHVEECSCYPRYPGVRCVCRDNWRGSNRPIVDINIKDYSIVSRYVCSGLVGDTPRKNDSSSSSHCLDPNNEEGGHGVKGWAFDDGNDVWMGRTISEKSRLGYETFKVIEGWSNPKSKLQTNRQVIVDRGNRSGYSGIFSVEGKSCINRCFFVELIRGRKEETEVLWTSNSIVVLCGTSGTYGAGSWPDGADLNLMPI

SEQ ID NO: 41 - protein, 469 aa, synthetic construct
MNPNQKIITIGSVSLTISTICFFMQIAILITTVTLHFKQYEFNSPPNNQVMLCEPTIIERNITEIVYLTNTTIEKEICPKPAEYRNWSKPQCGITGFAPFSKDNSIRLSAGGDIWVTREPYVSCDPDKCYQFALGQGTTLNNVHSNNTVRDRTPYRTLLMNELGVPFHLGTKQVCIAWSSSSCHDGKAWLHVCITGDDKNATASFIYNGRLVDSVVSWSKEILRTQESECVCINGTCTVVMTDGSASGKADTKILFIEEGKIVHTSTLSGSAQHVEECSCYPRYPGVRCVCRDNWKGSNRPIVDINIKDHSIVSSYVCSGLVGDTPRKNDSSSSSHCLDPNNEEGGHGVKGWAFDDGNDVWMGRTINETSRLGYETFKVIEGWSNPKSKLQINRQVIVDRGDRSGYSGIFSVEGKSCINRCFYVELIRGRKEETEVLWTSNSIVVFCGTSGTYGTGSWPDGADLNLMPI

SEQ ID NO: 42 - protein, 469 aa, synthetic construct
MNPNQKIISIGSVSLTIATICFLMQIAILVTTVTLHFKQYECNSPPNNQVMPCEPIIIERNITEIVYLNNTTIEKEICPKLAEYRNWSKPQCDITGFAPFSRDNSIRLSAGGNIWVTREPYVSCDPGKCYQFALGQGTTLNNGHSNDTVHDRTPYRTLLMSELGVPFHLGTRQVCIAWSSSSCYDGKAWLHVCVTGDDENATASFIYDGRLVDSVVSWSKDILRTQESECVCIDGTCTVVMTDGNATGKADTKILFIKEGKIVHTSKLSGSAQHVEECSCYPRYPDVRCICRDNWKGSNRPIVDINVKDYSIVSSYVCSGLVGDTPRKSDSSSSSHCLNPNNEEGGHGVKGWAFDNGDDVWMGRTISEKSRLGYETFKVVEGWSNPKSKLQTNRQVIVDRGNRSGYSGIFSVESKSCINRCFFVELIRGRKQETEVWWTSNSIVVLCGTSGTYGAGSWPDGADINLMPI

SEQ ID NO: 43 - protein, 469 aa, synthetic construct
MNPNKKIITIGSVSLTIATVCFLMQIAILATTVTLHFKQHECNSPPNNQVVLCEPTIIERNMTEIVYLTNITIEKEICPKLVEYRNWSKPQCNITGFAPFSKDNSVRLSAGGDIWVAREPYVSCDPNKCYQFALGQGTTLNNRHSNDTVHDRIPHRTLLMNELGIPFHLGTKQVCMAWSSSSCHDGKAWLHVCVTGDDKNATASIIYNGRLVDSIGSWSKKILRTQESECVCINGTCAVVMTDGSASGRADTKILFIEEGKIVHISPLSGSAQHVEECSCYPRYPGVRCICRDNWKGSNRPIVDINIKDYSIVSRYVCSGLVGDTPRKNDRSSSSYCRNPNNEKGSHGVKGWAFDDGDDVWMGRTISEKLRSGYETFKVIEGWSKPNSKLQINRQVIVERGNRSGYSGIFSVEGKNCINRCFYVELIRGREQETRVWWTSNSIIVFCGTSGTYGTGPWPDGADLNLMHI

SEQ ID NO: 44 - protein, 584 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEAPGGPYKIGTSGSCPNITNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 45 - protein, 584 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHIRLSTHNVINAEAPGGPYKIGTSGSCPNITNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 46 - protein, 585 aa, synthetic construct
MKAIIVLLMVVTSSADRICTGITSSSSPHVVKTATQGEINVTGVIPLTTTPIKSHFANLKGTETRGKLCPKCPNCTDLDVALGRPKCTGKIPSARVSILHEARPVTSGCFPIMHDRTKIRQLPNLLRGYEHVRLSTHNVINAEDAPGGPYKIGTSGSCPNITNGNGFFATMAWAVPKNDKNKTATNPLTIEVPYVCTEGEDQITVWGFHSDNEAQMAKLYGDSKPQKFTSSANGVATHYVSQIGGFPNQTEDGGLPQSGRIIVDYMVQKPGKTGTIVYQRGILLPQKVWCTSGRSKVIKGFLPLIGEADCLHEKYGGLNKSRPYYTGEHAKAVGNCPIWVKTPLKLANGTKYRPPAKLLKEKGFFGAIAGFLEGGWEGMVAGWHGYTSHGAHGIAVAADLKSTQEAINRITKNLNSLSELEVKNLQRLSSAMDELHNEILELDEKVDDLRADTISSQIAEAVHFSNEGIINSEDEHLMALERKLKKMLGPSAVEIGNGCFETQHKCNQTCLDKIAAGTFDAEEFSLPTFDSLNITAASLDDDGLDNHTILLYYSTAASSLAVTLMIAIFIVYMISRDNVSCSICL

SEQ ID NO: 47 - protein, 583 aa, synthetic construct
MKAMIVLLMVVTSNADRICTGITSSNSPHIVKTATQGEVNVTGVIPLTTTPTKSHFANLKGTKTRGKLCPKCLNCTDLDVALGRPKCTGNIPSARVSILHEVRPVTSGCFPIMHDRTKITQLPNLLRGYEHIRLSTHNVINAENAPGGPYKIGTSGSCPNVTNGNGFFATMAWAVPDKNKTATNSLTIEVPYICTEGEDQITIWGFHSDNETQMAKLYGDSNPQKFTSSANGITTHYVSQIGGFPDQTEDGGLPQSGRIVVDYMVQKSGKTGTITYQRGILLPQKVWCVSGRSKVIKGSLPLIGEADCLHEKYGGLNKSKPYYTGEHAKAIGNCPIWVKTPLTLANGTKYRPPAKLLRERGFFGAIAGFLEGGWEGMIAGWHGYTSHGAHGVAVAADLKSTQEAINKITQNLNSLSELEIKNLQRLSGAMDELHNEIIELDEKVDDLRADTISSQIELTVLLSNEGIINNEDEHLLALERKLKKMLGPSAIEIGNGCFETKHKCNQTCIDRIAAGTFDAGEFSLPTFDSLNITAASLNDDGLDNHTIMLYYSTAASSLAVTLMIAIFVVYMVSRDNFSCSICL

SEQ ID NO: 48 - protein, 584 aa, synthetic construct
MKTIIVLLMVVTSKADRICTGITSSNSPHVVKTATQGEVNVTGAIPLTTTPTKSYFANLKGTRTRGKLCPDCLNCTDLDVALGRPMCVGTTPSAKASILHEVRPATSGCFPIMHDRTKIRQLANLLRGYENIRLSTQNVIDAEKAPGGPYRLGTSGSCPNATSKSGFFATMAWAVPKDNNKNATNPLTVEVPYICTEGEDQITVWGFHSDNKTQMKNLYGDSNPQRFTSSANGVTTHYVSQIGDFPDQTEDGGLPQSGRIVVDYMMQKPGKTGTIVYQRGVLLPQKVWCASGRSKVIKGSLPLIGEADCLHEEYGGLNKSKPYYTGKHAKAIGNCPIWVKAPLKLANGTKYRPPAKLLKERGFFGAIAGFLEGGWEGMITGWHGYTSHGAHGVAIAADLKSTQEAINKITKNLNSLSELEVKNLQRLSGAMNELHNEILELDEKVDDLRADTISSQIELAVLLSNEGIINSEDEHLLALERRLKKMLGPSAVDIGNGCFETKHKCNQTCLDRIAAGTFNAGEFSLPTFDSLNITAASLNEDGLDNHTILLYYSTVASSLAVTLMLAIFIVYMVSRDNVSCSVCL

SEQ ID NO: 49 - protein, 585 aa, synthetic construct
MKAIIVLLMVVTSNADRICTGITSSNSPHVVKTATQGEVNVTGVIPLTTTPTKSHFANLKGTETRGKLCPKCLNCTDLDVALGRPKCTGKIPSARVSILHEVRPVTSGCFPIMHDRTKIRQLPNLLRGYEHVRLSTHNVINAEDAPGGPYKIGTSGSCPNITNGNGFFATMAWAVPKNDKNKTATNPLTIEVPYICTEGEDQITVWGFHSDNETQMAKLYGDSKPQKFTSSANGVTTHYVSQIGGFPNQTEDGGLPQSGRIVVDYMVQKSGKTGTITYQRGILLPQKVWCASGRSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVEIGNGCFETKHKCNQTCLDRIAAGTFDAGEFSLPTFDSLNITAASLNDDGLDNHTILLYYSTAASSLAVTLMIAIFVVYMVSRDNVSCSICL

SEQ ID NO: 50 - protein, 584 aa, synthetic construct
MKTIIVLLMVVTSSADRICTGITSSNSPHIVKTATQGEVNVTGAIPLTTTPTKSYFANLKGTRTRGKLCPDCLNCTDLDVALGRPMCVGTTPSAKASILHEVRPVTSGCFPIMHDRTKIRQLANLLRGYENIRLSTQNVIDAEKAPGGPYRLGTSGSCPNATSKSGFFATMAWAVPKDNNKNATNPLTVEVPYVCTEGEDQITVWGFHSDNKTQMKNLYGDSNPQKFTSSANGITTHYVSQIGGFPDQTEDGGLPQSGRIVVDYMMQKPGKTGTIVYQRGVLLPQKVWCVSGRSKVIKGSLPLIGEADCLHEEYGGLNKSKPYYTGKHAKAIGNCPIWVKTPLKLANGTKYRPPAKLLRERGFFGAIAGFLEGGWEGMIAGWHGYTSHGAHGIAVAADLKSTQEAINKITQNLNSLSELEIKNLQRLSGAMNELHNEILELDEKVDDLRADTISSQIELAVLLSNEGIINSEDEHLMALERKLKKMLGPSAVDIGNGCFETKHKCNQTCIDRIAAGTFNAGEFSLPTFDSLNITAASLDDDGLDNHTILLYYSTAASSLAVTLMLAIFIVYMVSRDNVSCSICL

SEQ ID NO: 51 - protein, 584 aa, synthetic construct
MKTAIIVLLMVVTSKADRICTGITSSSSPHVVKTATQGEINVTGVIPLTTTPIKSHFANLKGTKTRGKLCPKCPNCTDLDVALGRPKCTGNIPSARVSILHEARPVTSGCFPIMHDRTKIRQLPNLLRGYEHIRLSTHNVINAENAPGGPYKIGTSGSCPNVTNGNGFFATMAWAVPDKNKTATNSLTIEVPYICTEGEDQITIWGFHSDNEAQMAKLYGDSKPQKFTSSANGVATHYVSQIGDFPDQTEDGGLPQSGRIVVDYMVQKPGKTGTIVYQRGILLPQKVWCVSGRSKVIKGSLPLIGEADCLHEKYGGLNKSKPYYTGEYAKAIGNCPIWVKTPLKLANGTKYRPPAKLLKEKGFFGAIAGFLEGGWEGMIAGWHGYTSHGAHGVAVAADLKSTQEAINKITKNLNSLSELEVKNLQRLSSAMDELHNEIIELDEKVDDLRADTISSQIELAVLLSNEGIINSEDEHLMALERKLKKMLGPSAIEIGNGCFETKHKCNQTCLDKIAAGTFDAGEFSLPTFDSLNITAASLNEDGLDNHTILLYYSTAASSLAVTLMIAIFIVYMISRDNVSCSICL

SEQ ID NO: 52 - protein, 586 aa, synthetic construct
MKTAIIVLLMVVTSNADRVCTGITSSNSPHVVKTATQGEINVTGVIPLTTTPTRSHFANLKGTKTRGKLCPTCLNCTDLDVALGRPMCVGITPSAKASILHEVKPVTSGCFPIMHDRTKIRQLPNLLRGYENVRLSTHNVINAEGAPGGPYKIGTSGSCPNATSKIGFFATMAWAVPKNDNNKTATNPLTVEVPYICTEGEDQITVWGFHSDNKTQMKSLYGDSNPQKFTSSANGMTTHYVSQIGSFPDQTEDGGLPQSGRIIVDYMVQKSRKTGTITYQRGILLPQKVWCVSGRSKVIKGSLPLIGEADCLHEKYGGLNKSKPYYTGEHAKAVGNCPIWVKTPLKLANGTKYRPPAKLLRERGFFGAIAGFLEGGWEGMIAGWHGYTSHGAHGVAVAADLKSTQEAINKITKNLNSLSELEVKNLQRISGAMDELHNEILELDEKVDDLRADTISSQIELAVLLSNEGIINSEDEHLMALERKLKKMLGPSAIEIGNGCFETKHKCNQTCIDRIAAGTFNAGEFSLPTFDSLNITAASLNDDGLDSHTILLYYSTAASSLAVTLMIAIFVVYMISRDNVSCSICL

SEQ ID NO: 53 - protein, 585 aa, synthetic construct
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 54 - protein, 584 aa, synthetic construct
MKAILVLLMVVTSNADRICTGITSSNSPYVVKTATQGEVNVTGAIPLTTTPTKSHFANLKGTKTRGKLCPKCLNCTDLDVALGRPKCTGNIPSAEVSILHEVRPVTSGCFPIMHDRTKIRQLANLLRGYENIRLSTQNVIDAEKAPGGPYKLGTSGSCPNATSKSGFFATMAWAVPKDNNKNATNPLTVEVPYICAEGEDQITVWGFHSDNKTQMKNLYGDSNPQKFTSSANGVTTHYVSQIGGFPDQTEDGGLSQSGRIVVDYMMQKPGKTGTITYQRGILLPQKVWCASGRSKVIKGSLPLIGEADCLHEKYGGLNKSKPYYTGEHAKAIGNCPIWVKTPLKLANGTKYRPPARLLKERGFFGAIAGFLEGGWEGMVAGWHGYTSHGAHGVAVAADLKSTQEAVNKITKNLNSLSELEVKNLQRLSGAMDELHDEILELDEKVDDLRADTISSQIELAVVLSNEGIINSEDEHLLSLERKLKKMLGPSAVEIGNGCFETKHKCNQTCLDRIAAGTFDAGEFSLPTFDSLNITAASLNDDGLDNHTILLYYSTAASSLTVTLMLAIFIVYMVSRDNISCSICL

SEQ ID NO: 55 - protein, 584 aa, synthetic construct
MKAIIVLLMVVTSDADRICTGITSSNSPHVVKTATQGEINVTGVIPLTTTPTKSYFANLKGIRTRGKLCPDCINCTDLDVALGRPMCMGTTPSAKASILHEVRPITSGCFPIMHDRTKIRQLPNLLRGYEKIRLSTQNVIDAEKAPRGPYRLGTSGSCPNATSKIGFFATMAWAVPKDNYKNATNPQTVEIPYICTEGEDQITVWGFHSDDKTQMKSLYGDSNPQKFTSSANGMTTHYVSQIGDFPDQTEDGRLPQSGRIVVDYMVQKPEKTGTIVYQRGVLLPQKVWCVSGRSKVIKGALPLIGEADCLHEEYGGLNKSKPYYTGKHAKAIGDCPIWVKTPLELANGTKYRAPAKLLKERGFFGAIAGFLEGGWKGMIAGWHGYISHGAHGVAVAADLKSTQEAINKITRNLNSLSELEVKNLQRLSGAMDELHSEILELDEKVDDLRADTVSSQIELAVLLSNEGIIKSEDEHLLALERKLKKMLGPSAIDIGNGCFETTHKCNQTCLDRIAAGTFNAREFSLPTFDSLNITAAYLNDDGLDNHAILLYYSTAASSLAVTLMLAIFIVYVVSRDNVSCSICL

SEQ ID NO: 56 - protein, 584 aa, synthetic construct
MKVIIVLLMVVMSNADRICTGITSSSSPHVVKTATQGEVNVTGVIPLTITPTKSYFANLKGTRTRGKLCPDCLNCTDLDVALGRPMCVGTTPSAKASILHEVKPVTSGCFPIMHDRTKIRQLPNFLRGYEKIRLSTKNVIDAEKAPGGPYRLGTSRSCPNATSKVGFFATMAWAVPKNNYKNATNPQTVEVPYICTEGEDQITVWGFHSDNKTQMKSLYGDSNPQRFTSSANGVTTHYVSQIGSFPDQTEDGGLPQSGRIVVDYMVQKPGKTGTIVYQRGVLFPQKVWCASGKSKVIKGSLPLIGEADCLHEEYGGLTKSKPYYTGKHAKAIGNCPIWVKTSLKLANGTKYRPPAKLLKERGLFGAIAGFLEGGWEGMIAGWHGYTSHGAHGVAVAADLKSTQEAINKITKNLNFLSELEVKNLQRLSGAMDELHNEILELDEKVDDLRADTMSSQIELAVLLSNEGIINSEDEHLLALERQLKKMLGPSAVDIGNGCFETKHKCNQTCLDKIAAGTFNAGEFSLPTFDSLNITAASLNNDGLDNHTILLYYSTVASSLAVTLMIAIFVVYMVSRDNVSCSICL

SEQ ID NO: 57 - protein, 584 aa, synthetic construct
MKAIIVLLMVVTSNADRICTGITSSNSPHVVKTATQGEVNVTGVIPLTTTPTKSYFANLKGTRTRGKLCPDCLNCTDLDVALGRPMCVGTTPSAKASILHEVRPVTSGCFPIMHDRTKIRQLPNLLRGYEKIRLSTQNVIDAEKAPGGPYRLGTSGSCPNATSKIGFFATMAWAVPKDNYKNATNPQTVEVPYICTEGEDQITVWGFHSDNKTQMKSLYGDSNPQKFTSSANGVTTHYVSQIGDFPDQTEDGGLPQSGRIVVDYMVQKPGKTGTIVYQRGVLLPQKVWCASGRSKVIKGSLPLIGEADCLHEEYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHNEILELDEKVDDLRADTISSQIELAVLLSNEGIINSEDEHLLALERKLKKMLGPSAVDIGNGCFETKHKCNQTCLDRIAAGTFNAGEFSLPTFDSLNITAASLNDDGLDNHTILLYYSTAASSLAVTLMLAIFIVYMVSRDNVSCSICL

SEQ ID NO: 58 - protein, 583 aa, synthetic construct
MKVIIVLLMVVTSDADRICTGITSSNSPHVVKTATQGEVNVTGVIPLTITPTKSYFANLKGTKTRGKLCPDCFNCTDLDVALGRPMCMGTTPSAKASILHEVKPVTSGCFPIMHDRTKIRQLANLLRGYENIRLSTQNVIDAEKAPRGPYRLGTSRSCPNATSKSGFFATMAWAVPKDNNKNATNPLTVEVPYICAEGEDQITVWGFHSDDKTQMKNLYGDSNPQRFTSSANGVTTHYVSQIGGFPDQTEDGGLPQSGRIVVDYMMQKPGKTGTIVYQRGVLFPQKVWCASGKSKVIKGSLPLIGEADCLHEKYGGLNKSKPYYTGEHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHSEILELDEKVDDLRADTISSQIELAVLLSNEGIINSEDEHLLALERKLKKMLGPSAVEIGNGCFETKHKCNQTCLDKIAAGTFNAGEFSLPTFDSLNITAASLNNDGLDNHTILLYYSTAASSLTVTLMLAIFIVYVVSRDNVSCSIC

SEQ ID NO: 59 - protein, 583 aa, synthetic construct
MKAIIVLLMVVMSNADRICTGITSSNSPHVVKTATQGEVNVTGVIPLTTTPTKSHFANLKGTKTRGKLCPDCINCTDLDVALGRPMCVGTIPSAKASILHEVRPVTSGCFPIMHDRTKIRQLPNLLRGYEKIRLSTKNVIDAEKAPRGPYRLGTSGSCPNATSKVGFFATMAWAVPKNNYKNATNPQTVEIPYICTEGEDQITVWGFHSDNKIQMKNLYGDSNPQKFTSSANGVTTHYVSQIGSFPDQTEDGGLPQSGRIVVDYMVQKPEKTGTIVYQRGILLPQKVWCVSGRSKVIKGALPLIGEADCLHEKYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHDEILELDEKVDDLRADTISSQIELAVLLSNEGIINSEDEHLLALERKLKKMLGPSAIDIGNGCFETKHKCNQTCLDRIAAGTFDAGEFSLPTFDSLNITAASLNEDGLDNHTILLYYSTVASSLAVTLMIAIFVVYMVSRDNVSCSIC

SEQ ID NO: 60 - protein, 584 aa, synthetic construct
MKAIIVLLMVVASDADRICTGITSSNSPHVVKTATQGEVNVTGVIPLTITPTKSYFANLKGIRTRGKLCPDCFNCTDLDVALGRPMCVGATPSAKASILHEVRPVTSGCFPIMHDRTKIRQLPNLLRGYENIRLSTQNVIDAEKAPRGPYRLGTSGSCPNTTSKIGFFATMAWAVPKDKYKNATNPQTLEIPYICTEGEDQITVWGFHSDNKTQMKNLYGDSNPQKFTSSANGVTTHYVSQIGDFPNQTEDGGLPQSGRIVVDYMVQKPGRTGTIVYQRGILLPQKVWCANGRSKVIKGALPLIGEADCLHEKYGGLNKSKPYYTGKHAKAIGNCPIWVKTPLKLANGTKYRPPAKLLKERGFFGAIAGFLEGGWEGMIAGWHGYTSHGAHGVAVAADLKSTQEAINKITKNLNSLSELEVKNLQRLSGAMDELHSEILELDEKVDDLRADTISSQIELAVLLSNEGIINSEDEHLLALERKLKKMLGPSAIDIGNGCFETKHKCNQTCLDRIAAGTFSAGEFSLPTFDSLNITAASLNDDGLDNHAILLYYSTAASSLTVTLMLAIFIVYMVSKDNVSCSICL

SEQ ID NO: 61 - protein, 467 aa, synthetic construct
MLPSTIQTLTLFLTSGGVLLSLYVSASLSYLLYSDILLKFSPTEITAPTMPLDCANASNVQAVNRSATKGVTLLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSKTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 62 - protein, 466 aa, synthetic construct
MLPSTIQTLTLVLTSGGVLLSLYVSALLSYLLYSDILLKFSRTEITAPIMPLDCANASNVQAVNRSATKGVTLLLPGPEWTYPRLSCQGSTFQKALLISPHRFGEAKGNSAPLIIREPFIACGPKECKHFALTHYAAQPGGYYNGTREDRNKLRHLISVNLGKIPTVENSIFHMAAWSGSACHDGREWTYIGVDGPDSNALLKIKYGEAYTDTYHSYAKNILRTQESACNCIGGDCYLMITDGPASGISECRFLKIRKGRIIKEIFPTGRVEHTEECTCGFASNKTIECACRDNSYTAKRPFVKLNVEIDTAEIRLMCTETYLDTPRPDDGSITGPCESNGDKGSGGIKGGFVHQRMESKIGRWYSRTMSKTERMGMGLYVKYDGDPWADSGALAFSGVMVSMKEPGWYSFGFEIRDKKCDVPCIGIEMVHDGGRETWHSAATTIYCLMGSGQLLWDTVTGVNMTL

SEQ ID NO: 63 - protein, 466 aa, synthetic construct
MLSSTIQTLTLLLTSGGVLLSLYVSASLSYLLYSDILLKFSQTEITAPIMSLDCANASNVQAVNRSAAKGVTLLLPEPEWTYPRLSCPGSIFQKALLISPHRFGEIKGNSAPLIIREPFIACGPTECKHFALTHYAAQPGGYYSGTRGDRNKLRHLISVKLGKIPTVETSIFHMAAWSGSSCHDGKEWTYIGVDGPDNNALLKIKYGEAYTDTYHSYANKILRTQESACNCIGGNCYLMITDGPASGVSECRFLKIREGRIIKEIFPTGRIKHTEECTCGFASNETIECACRDNNYTAKRPFVKLNVETDTAEIRLMCTKTYLDTPRPNDGSTTGPCESDGDKGSGGIKGGFVHQRMASKIGRWYSRTMSKTKRMGMGLYVKYDGDPWTDSEALALSGVMVSMEEPGWYSFGFEIKDKKCDVPCIGIEMVHDGGKTTWHSAATAVYCLMGSGQLLWDTVTGVDMAL

SEQ ID NO: 64 - protein, 466 aa, synthetic construct
MLPSTIQTLTLFLTSGGVLLSIYVSASLSYLIYSDILLKFSPTEITAPTMPLDCANASNVHAVNRSATKGVTFLLPEPEWTYPRLSCPGSTFQKALLISPHRFGETKGNSAPLIIREPFVACGPNECKHFALTHYATQPGGYYNGTRGDRNKLRHLISVELGKIPTVENSIFHMTAWSGSACHDGKEWTYVGVDGPDNNALLKVKYGEAYTDTYHSYANNILRTQESACNCIRGNCYLMITDGSASGVSECRFLKIQEGRIIKEIFPTGRVKHTEECTCGFANNKTIECACRDNRYTAKRPFVKLSVETDTAEIRLMCTDTYLDTPRPNDGSITGPCESDGNEGSGGIKGGFVHQRMKSKIGRWYSRTMSQTERMGMGLYVKYGGDPWADSDALAFSGVMISMKEPGWYSFGFEMKDKKCDVPCIGVEMVHDGGKETWHSAATAIYCLMGSGKLLWDTVTGVNMAL

SEQ ID NO: 65 - protein, 466 aa, synthetic construct
MLPSTIQTLTLFLTSGGVLLSLYVSASLSYLLYSDILLKFSPTEITAPTMPLDCANASNVQAVNRSATKGVTLLLPEPEWTYPRLSCPGSTFQKALLISPHRFGETKGNSAPLIIREPFVACGPNECKHFALTHYAAQPGGYYNGTRGDRNKLRHLISVKLGKIPTVENSIFHMAAWSGSACHDGKEWTYIGVDGPDNNALLKVKYGEAYTDTYHSYANNILRTQESACNCIGGNCYLMITDGSASGVSECRFLKIREGRIIKEIFPTGRVKHTEECTCGFASNKTIECACRDNRYTAKRPFVKLNVETDTAEIRLMCTDTYLDTPRPNDGSITGPCESDGDKGSGGIKGGFVHQRMKSKIGRWYSRTMSKTERMGMGLYVKYGGDPWADSDALAFSGVMVSMKEPGWYSFGFEIKDKKCDVPCIGIEMVHDGGKETWHSAATAIYCLMGSGQLLWDTVTGVDMAL

SEQ ID NO: 66 - protein, 466 aa, synthetic construct
MLSSTIQTLTLVLTSGGVLLSLYVSALLSYLLYSDILLKFSQTEITAPIMPLDCANAPNVQAVNRSAAKGVTLLLPGPEWTYPRLSCQGSTFQKALLISPHRFGEIKGNSAPLIIREPFIACGPKECKHFALTHYATQPGGYYNGTREDRNKLRHLISVNLGKIPTVENSIFHMTAWSGSACHDGREWTYIGVDGPDSNALLKIKYGEAYTDTYHSYAKNILRTQESACNCIGGDCYLMITDGPASGISECRFLKIQEGRIIKEIFPTGRIKHTEECTCGLASNETIECACRDNSYTAKRPFVKLNVEIDTAEIRLMCTETYLDTPRPDDGSITGPCESNGDKGSGGIKGGFVHQRMASKIGRWYSRTMSKTKRMGMGLYVKYDGDPWTDSEALALSGVMVSMEEPGWYSFGFEIRDKKCDVPCIGVEMVHDGGKTTWHSAATAVYCLMGSGQLLWDTVTGVNMTL

SEQ ID NO: 67 - protein, 466 aa, synthetic construct
MLPSTIQMLTLFLTSGGVLLSIYVSASLSYLIYSDILLKFSRTEITAPIMSLDCANASNVHAVNRSATKGVTFLLPEPEWTYPRLSCSGSTFQKALLISPHRFGEAKGNSAPLIVREPFIACGPTECKHFALTHYAAQPGGYYSGTRGDRNKLRHLISVELGKIPTVETSIFHMAAWSGSSCHDGKEWTYVGVDGPDNNALLKIKYGEAYTDTYHSYANKILRTQESACNCIRGNCYLMITDGPASGVSECRFLKIREGLIIKEIFPTGRVEHTEECTCGFANNKTIECACRDNNYTAKRPFVKLSVETDTAEIRLMCTKTYLDTPRPNDGSTTGPCESDGEKGSGGIKGGFVHQRMESKIGRWYSRTMSQTERMGMGLYVKYDGDPWADSGALAFSGVMISMKEPGWYTFGFEIKDKKCDVPCIGIEMVHDGGRETWHSAATTIYCLMGSGKLLWDTVTGVNMAL

SEQ ID NO: 68 - protein, 466 aa, synthetic construct
MLPSTIQTLTLFLTSGGVLLSLYVSASLSYLLYSDILLKFSRTEVTAPIMPLDCANASNVQAVNRSATKGVTPLLPEPEWTYPRLSCPGSTFQKALLISPHRFGETKGNSAPLIIREPFIACGPKECKHFALTHYAAQPGGYYNGTREDRNKLRHLISVKLGKIPTVENSIFHMAAWSGSACHDGREWTYIGVDGPDSNALLKIKYGEAYTDTYHSYAKNILRTQESACNCIGGDCYLMITDGPASGISECRFLKIREGRIIKEIFPTGRVKHTEECTCGFASNKTIECACRDNSYTAKRPFVKLNVETDTAEIRLMCTKTYLDTPRPNDGSITGPCESDGNEGSGGIKGGFVHQRMASKIGRWYSRTMSKTQRMGMGLYVKYDGDPWTDSEALALSGVMVSMEEPGWYSFGFEIKDKKCDVPCIGIEMVHDGGKTTWHSAATAIYCLMGSGQLLWDTVTGVNMTL

SEQ ID NO: 69 - protein, 466 aa, synthetic construct
MPPSTIQTLTLFLTSGGVLLSIYVSASLSYLLYLDILLKFPSTEITAPTMPLDCANASNVQAVNHSATKGVTLLLPEPEWTYPRLSCPGSTFQRALLISPHRFGETKGNSAPLIMREPFIACGPNECKHFALTHYTAQPGGYYNGTREDRNNLRHLISVKLGKIPTVENSIFHMAAWSGSACHDGKEWTYIGVDGPDNNALLKVKYGEAYTDTYHSYANKILRTQESACNCIGGNCYLMITDGSASGVSECRFLKIREGRIMKEIFPTGRTQHTEECTCGFASNKTIECACRDNSYTAKRPFVKLNVETDTAEIRLMCTETYLDTPRPDDGSITGPCESDGDEGSGGIKGGFVHQRMESKIGRWYSRTMSKTERKGMGLYVKYNGDPWADSDALAFSGVMVSMKEPGWYSFGFEIKDKKCDVPCIGIEMVHDGGKETWHSAATAIYCLMGSGQLLWDTVTGVDMAL

SEQ ID NO: 70 - protein, 466 aa, synthetic construct
MLPSTIQTLTLLLTSGGVLLSLYVSASLSYLLYSDILLKFSRTEVTAPIMPLDCANASNVQAVNRSVTKGVTPLLPEPEWMYPRLSCPGSTFQKALLISPHRFGEIKGNSAPLIIREPFIACGPKECKHFALTHYATQPGGYYNGTREDRNKLRHLVSVKLGKIPTVENSMFHMAAWSGSACHDGREWTYIGVDGPDNNALLKIKYGETYTDTYHSYAKNILRTQESACNCIGGDCYLMITDGPASGVSECRFLKIREGRIVKEIFPTGRVKHTEECTCGFANNKTIECACRDNRYTAKRPFVKLNMETDTAEIRLMCTKTYLDNPRPNDGSITGPCESDGNEGSGGIKGGFVHQRMASKIARWYSRTMSKTKRMGMGLYVKYDGDPWTDSEALALSGVMISMEEPGWYSFGFEIRDKKCDVPCIGVEMVHDGGKKTWHSAATAIYCLMGSGQPLWDTVTGVNITL

SEQ ID NO: 71 - protein, 466 aa, synthetic construct
MLPTTIQTLTLFLTSGGVLLSLYMSASLSYLLYSDMLLKFSRTEVTAPMMPLDCANASNVQAVNRSATKGVTPLLPEPEWTYPRLSCQGSTFQKALLISPHRFGETRGNSAPLIIREPFVACGPKECKHFALTHYAAQPGGYYNGTRGDRNKLRHLISVKLGKVPTVENSMFHMTAWSGSACHDGREWIYIGVDGPDSNALLKIKYGEAYTDTYHSYANNILRTQESACNCIRGDCYLMITDGPASGISECRFLKIREGRIIKEIFPTGRVEHTEECTCGFASNKTIECACRDNNYTAKRPFVKLNVETDTAEIKLMCTKTYLDTPRPNDGSITGPCESDGKEGSGGIKGGFVHQRMASKIGRWYSRTMSKTQRMGMGLYVKYDGDPWTDSDALALSGVMVSMEEPGWYSFGFEVKDKKCDVPCIGMEMVHDGGKTTWHSAATAVYCLMGSGQLLWDTVTGVNMTL

SEQ ID NO: 72 - protein, 466 aa, synthetic construct; identical to SEQ ID NO: 68

SEQ ID NO: 73 - protein, 466 aa, synthetic construct
MLSSTIQTLTLLLTSGGVLLSLYVSALLSYLLYSDMLLKFSRTEVTAPMMPLDCANASNVQAVNRSVTKGVTPLLPEPEWMYPRLSCPGSTFQRALLISPHRFGEIKGNSAPLIIREPFVACGPKECKHFALTHYTAQPGGYYNGTRGDRNKLRHLVSVKLGKIPTVENSMFHMAAWSGSACHDGKEWTYIGVDGPDNNALLKIKYGETYTDTYHSYANNILRTQESACNCIGGNCYLMITDGSASGVSECRFLKIREGRIVKEIFPTGRTQHTEECTCGFANNKTIECACRDNRYTAKRPFVKLNVEIDTAEIRLMCTETYLDTPRPDDGSITGPCESDGKEGSGGIKGGFVHQRMESKIGRWYSRTMSKTKRMGMGLYVKYNGDPWADSDALALSGVMISMEEPGWYSFGFEIRDKKCDVPCIGVEMVHDGGKETWHSAATAVYCLMGSGQPLWDTVTGVDMAL

SEQ ID NO: 74 - protein, 466 aa, synthetic construct
MLPTTIQTLTLFLTTGGVLLSLYMSASLSYLLYLDILLKFPSTEITAPTMPLDCANASNAQAVNRSATKGVTLLLPEPEWTYPRLSCQGSTFQKALLISSHRFGETKGNSAPLIMREPFIACGPNECKHFALTHYATQPGGYYNGTREDRNNLRHLISVKLGKVPTVENSMFHMTAWSGSACHDGREWIYIGVDGPDNNALLKVKYGEAYTDTYHSYANKILRTQESACNCIRGDCYLMITDGPASGVSECRFLKIREGRIMKEIFPTGRVEHTEECTCGFASNKTIECACRDIPYTAKRPFVKLNVETDTAEIKLMCTKTYLDNPRPNDGSITGPCESDGDEGSGGIKGGLVHQRMASKIARWYSRTMSKTERKGMGLYVKYDGDPWTDSDALALSGVMVPMEEPGWYSFGFEIKDRKCDVPCIGLEMVHDGGKKTWHSAATAIYCLMGSGRLLWDTVTGVNITL

SEQ ID NO: 75 - protein, 548 aa, synthetic construct
MMKAILVLLTFTAAYADTCIGYHANNSTDTVDTVLEKNVTVTHSVNLLEDHNGKLCKLGAPLLGKCNIAGWILGNPECESLTASWSYIVETSNSDNGTCYPGDFDYEELREQLSSVSSFERFEIFPKSSWPNHTGVTAACHNGSFYRNLIWLVKKGKENSYPKLSKSYNNKKEVLVLWGXXIHHPSTDQQLYQNAAYVFVGSSYSRKFPEIARPKVRDQGRMNYYWTLEPGDTITFEATGNLVAPRYAFALRGGSGIISDAPVHDCDTCQTPGAINTSLTSLPFQNVHPVTIGECPKYVKSTKLRMATGLRNIPSIQSRGLFGAIAGFIEGGWTGMIDGWYGYHHQNEQGSGYAADQKSTQNAIDGITNKVNSVIEKMNTQFTAVGKEFNLERRIENLNKKVDDGFLDIWTYNAELLVLLENERTLDFHDSNVKNLYEKVRSQLKNNAKEIGNGCFEFYHKCDNCMESVKNGTYDYPKYSEESKLNREEIDGVKLESRIYQILAIYSTVASSLVLLVSLGAISFWMCSNGSLQCRICIKNRDMRKNTL

SEQ ID NO: 76 - protein, 566 aa, synthetic construct
MKAILVVLLYTFTTANADTLCIGYHANNSTDTVDTVLEKNVTVTHSVNLLENRHNGKLCKLRGVAPLHLGKCNIAGWLLGNPECESLSTASSWSYIVETSNSDNGTCYPGDFINYEELREQLSSVSSFERFEIFPKTSSWPNHDTNRGVTAACPHAGTNSFYRNLIWLVKKENSYPKINKSYINNKEKEVLVLWAIHHPSTSADQQSLYKNADAYVFVGSSRYSRKFEPEIATRPKVRDQAGRMNYYWTLLDQGDTITFEATGNLVAPRYAFALKRNSGSGIIISDTSVHDCDTTCQTPNGAINTSLPFQNIHPVTIGECPKYVKSTKLRMATGLRNIPSVQSRGLFGAIAGFIEGGWTGMIDGWYGYHHRNEQGSGYAADLKSTQNAIDGITNKVNSVIEKMNTQFTAVGKEFSHLERRIENLNKKVDDGFLDIWTYNAELLVLLENERTLDYHDSNVKNLYEKVRSQLKNNAKEVGNGCFEFYHKCDDTCMESVKNGTYDYPKYSEEAKLNREEIDGVKLESTRIYQILAIYSTVASSLVLVVSLGAISFWMCSNGSLQCKICI

SEQ ID NO: 77 - protein, 565 aa, synthetic construct
MKVKLLILLCTFTATYADTICIGYHANNSTETVDTVLEKNVAVTHSVNLLEDSHNGKLCLLKGIAPLQLGNCSVAGWILGNPECESLISKKSWSYIVETPNPENGACYPGEFADYEELREQLSSVSSFKRFEIFPKESSWPNHTVTGVSASCSHNGERSFYRNLIWLTVKNGLYPNLSKSYENDKGKEVLILWGVHHPSNIGDQRTLYHTENAYVSVVSSHYSRRFTPEITKRPKVRDQEGRINYYWTLLEPGDTIIFEANGNLIAPWYAFALSRGLGSGIITSNAPMDECDAKCQTPQGAINSSLPFQNVHPVTIGECPKYVRSAKLRMVTGLRNIPSIQSRGLFGAIVGFIEGGWTGMVDGWYGYHHQNGQGSGYAADQESTQNAINGITNKVNSIIEKMNTQFTAVGKEFNKLERRMENLNKKVDDGLLDVWTYNAEMLVLLENERTLDFHDSNVKSLYEKVKSQLKNNAKEIGNGCFEFYHKCNNECMESVRNGTYDYPKYYEESKLNREKIDGVKLDSMGVYRILAIYSTAASSLVLLVSLGAISFWVCSNGSLQCRICI

SEQ ID NO: 78 - protein, 566 aa, synthetic construct
MEAKLFVLFCAFTALKADTICVGYHANNSTDTVDTILEKNVTVTHSVNLLEDKHNGKLCKLGGIAPLHLGKCNIAGWILGNPECELLISKESWSYIVETSSSDNGTCYPGDFIDYEELREQLSTVSSFERFEIFPKASSWPNHDSNKGVTAACPHAGAKSFYKNLIWLVKKGNSYPKLSKSYINDKGKEVLVLWGIHHPSTSADQQSLYQNADAYVFVGTSRYSKKFKPEIAIRPKVRDQEGRMNYYWTLVEPGDKITFEATGNLVVPRYAFAMERNAGSGIIISDTPVHDCNTTCQTPKGAINTSLPFQNIHPITIGKCPKYVKSTKLRLATGLRNVPSIQSRGLFGAIAGFLEGGWTGMVNGWYGYHHQNEQGSGYAADQKSTQIAIDGISNKVNSVIEKMNIQFTSVGKEFNHLEKRIENLNKKVDDGFLDVWTYNAELLILLENERTLDFHDFNVKNLYEKVKSQLRNNAKEIGNGCFEFYHKCDNECMESVKNGTYNYPKYSEESKLNREEIDGVKLESMGVYRILSIYSTVASSLVLLVSLGAVSFWMCSNGSLQCRVCI

SEQ ID NO: 79 - protein, 566 aa, synthetic construct
MKAILVVLLYTFTTANADTLCIGYHANNSTDTVDTVLEKNVTVTHSVNLLEDSHNGKLCKLRGVAPLHLGKCNIAGWLLGNPECESLSTASSWSYIVETSNSDNGTCYPGDFINYEELREQLSSVSSFERFEIFPKTSSWPNHDTNRGVTAACPHAGTNSFYRNLIWLVKKGNSYPKLSKSYINNKEKEVLVLWAIHHPSTSADQQSLYQNADAYVFVGSSRYSRKFEPEIATRPKVRDQAGRMNYYWTLVEPGDKITFEATGNLVVPRYAFALKRNSGSGIIISDTSVHDCDTTCQTPNGAINTSLPFQNIHPVTIGECPKYVKSTKLRMATGLRNIPSIQSRGLFGAIAGFIEGGWTGMIDGWYGYHHQNEQGSGYAADLKSTQNAIDGITNKVNSVIEKMNTQFTAVGKEFNKLERRMENLNKKVDDGFLDIWTYNAELLVLLENERTLDYHDSNVKNLYEKVRSQLKNNAKEIGNGCFEFYHKCNNECMESVKNGTYDYPKYSEEAKLNREEIDGVKLESTRIYQILAIYSTVASSLVLLVSLGAISFWMCSNGSLQCRICI

SEQ ID NO: 80 - protein, 565 aa, synthetic construct
MKVKLLILLCTFTATYADTICIGYHANNSTDTVDTILEKNVTVTHSVNLLENRHNGKLCLLKGIAPLQLGNCSVAGWILGNPECESLISKKSWSYIVETPNPENGACYPGEFADYEELREQLSTVSSFERFEIFPKESSWPNHTVTGVSASCSHNGERSFYRNLIWLTVKNGLYPNLSKSYENDKGKEVLILWGVHHPSNIGDQRTLYHTENAYVSVVSSHYSRRFTPEITKRPKVRDQEGRINYYWTLLEPGDTIIFEANGNLIAPWYAFALSRGLGSGIITSNAPMDECDAKCQTPQGAINSSLPFQNVHPVTIGECPKYVRSAKLRMVTGLRNVPSIQSRGLLGAIAGFIEGGWTGMVDGWYGYHHQNGQGSGYAADQESTQNAINGITNKVNSIIEKMNIQFTSVGKEFNHLEKRIENLNKKVDDGFLDVWTYNAELLILLENERTLDFHDSNVKSLYEKVKSQLKNNAKEVGNGCFEFYHKCDDTCMESVKNGTYNYPKYSEESKLNREKIDGVKLDSMGVYRILAIYSTVASSLVLVVSLGAISFWMCSNGSLQCRVCI

SEQ ID NO: 81 - protein, 566 aa, synthetic construct
MEAKLFVLFCAFTALKADTICVGYHANNSTETVDTVLEKNVAVTHSVNLLEDKHNGKLCKLGGIAPLHLGKCNIAGWILGNPECELLISKESWSYIVEAPNSDNGACYPGQFADYEELREHLSSVSSFEKFEIFPKASSWPNHDSNKGVTAACPYAGANSFYRNLLWIVKKGNSYPKINKSYINNKGKEVLVLWGIHHPPTSTDQQSLYKNADAYVFVGTSRYSKKFKPEIAIRPKVRDQEGRMNYYWTLLDQGDTITFEATGNLVAPRYAFAMERNAGSGIIISDTPVHDCNTTCQTPKGAINSNLPFQNVHPITIGECPKYVRSTKLRMVTGLRNIPSVQSRGLFGAIAGFLEGGWTGMVNGWYGYHHQNDQGSGYAADQKSTQIAIDGISNKVNSVIEKMNIQFTAVGKEFSHLERRIENLNKKVDDGLLDVWTYNAEMLVLLENERTLDFHDFNVKNLYEKVKSQLRNNAKEIGNGCFEFYHKCDNTCMESVRNGTYDYPKYYEESKLNREEIDGVKLESMGVYQILAIYSTAASSLVLLVSLGAVSFWMCSNGSLQCKICI

SEQ ID NO: 82 - protein, 571 aa, synthetic construct
MKTIIAFSCILCLIFAQKLPGSDNNSMATLCLGHHAVPNGTLVKTITDDQIEVTNATELVQSSSTGRICNSPGHQILDGKNCTLIDALLGDPHCDDFQNKWDLFVERSTAYSGNCYPYYVPDYASLREQLSLVASSGTLEFTQESFNWTGVAQDGSSYACRRGSVNNSFFSRLNWLYLNYKYPALNVTMPNNDKFDKLYIWGVHHPGTDKDQTNLYVQASGRVVSTKRSQQTVIPNIGSRPWVRGVSSIISIYWTIVKPGDILLINSTGNLIAPRGYFKIQSGKSSGIMRSDAPIDCNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQITGKLNRVIKKTNEKFHQIEKEFSEVEGRIQDLEKYVEDTKIDLWSYNAELLVALENQHTIDLTDSEMNKLFERTRKQLRENAEDMGNGCFKIYHKCDNACIGSIRNGTYDHDVYRDEALNNRFQIKGVQLKSGYKDWILWISFAISCFLLCVVLLGFISMWACQKGNIRCNICI

SEQ ID NO: 83 - protein, 566 aa, synthetic construct
MKTIIAFSCILCLIFAQKLPGSDNSTATLCLGHHAVPNGTMVKTITDDQVEVTNATELVQSSSTGRICNSPHQILDGKNCTLIDSLLGDPHCDDFQNKEWDLFVERSKAYSNCYPYYVPDYATLRSLVASSGNLEFTQESFNWTGVAQDGSSYACRRGSVNSFFSRLNWLYNLNYKYPEQNVTMPNNDKFDKLYIWGVHHPGTDKDQTNLYVQASGRVIVSTKRSQQTIIPNIGSRPWVRGVSSIISIHWTIVKPGDILLINSTGNLIAPRGYFKIQNGKSSIMRSDAHIDECNSECITPNGSISNDKPFQNVNKITYGACPRYIKQNTLKLATGMRNIPEKQTRGIFGAIAGFIENGWEGMVNGWYGFRHQNSEGTGQAADLKSTQAAINQITGKLNRVIKKTNEKFHQIEKEFSEVEGRIQDLEKYVEDTKIDIWSYNAELLVALENQHTIDLTDSEMSKLFERTRRQLRENAEDIGNGCFKIYHKCDNSCMESIRNGTYDHDIYRNEALNNRFQIKGVQLKSGYKDWILWISFAISCFLLCVVLLGFIMWACQRGNIRCNICV

SEQ ID NO: 84 - protein, 566 aa, synthetic construct
MKTIIALSYILCLVFAQKLPGNDNNMATLCLGHHAVPNGTIVKTITNDQIEVTNATELVQSFSTGEICNSPHQILDGENCTLIDALLGDPQCDGFQNNKWDLFVERSKAHSNCYPYDVPDYASLRSLVASSGTLEFNNESFNWTGVTQNGGSSACKRGPNNSFFSRLNWLTHLNFKYPALEVTMPNNEQFDKLYIWGVHHPATDKDQISLYAQAAGRIIVSTKRSQQAVIPNIGSRPRVRDIPSRISIYWTIVRPGDILLIKSTGNLIAPRGYFKIRSGKSSIMRSDAPIGKCNSACITPNGSIPNDKPFQNVNRITYGACPKYVKQNTLKLATGMRNIPERQTRGIFGAKAGFIENGWEGMVDGWYGFRHQNSEGRGQAADLKSTQAAIDQINGKLNRLIGKTNEKFHQIEKEFSEIEGRIQDLEKYIEDTKIDLWSYNAELLIALENQHTIDLADSEMNKLFEKTKKQLRENAEDMGNGCLKIYHKCDNACIGSIRNGTYDHDVYRDEALNNRFQIKGVELKSEYKDWILWISFAMSCFLLCVALLGFIMWACQKGNIKCNICI

SEQ ID NO: 85 - protein, 566 aa, synthetic construct
MKAIIAFSCILCQISAQKLPGSDNSMATLCLGHHAVPNGTLVKTITDDQIEVTNATELVQSTSKGEICSSPHQILDGKNCTLIDALLGDPHCDDFQNKKWDLFVERSTAYSNCYPYYVPDYASLRSLIASSGTLEFTQESFDWTGVAQNGSSYACRRGSVKSFFSRLNWLHNLNYKYPALNVTMPNNDNFDKLYIWGVHHPSTDSDQTSLYVQASGRVTVSTKRSQQTVIPNIGSRPWIRGVSSIISIYWTIVKPGDILIINSNGNLIAPRGYFKIQSGKSSIMRSDAPIGNCNSECITPNGSIPNDRPFQNVNRITYGACPRYVKQNTLKLATGMRNVPEKQTRGILGAIAGFIENGWEGMIDGWYGFRHHNSEGTGQAADIKSTQAAINQINGKLNRVIEKTNEKFHQIEKEFSGVEGRIQDLERYVEDTKIDLWSYNAELLVAMENQHTIDLTDSEMNKLFERTRKQLRENAEDMGNGCFKIYHKCDNTCIGSIRNGTYNHDVYRDEAVNNRFQIKGVQLKLGYKDWILWISFATSCFLLCAVLLGFIMWACQKGNIRCNICI

SEQ ID NO: 86 - protein, 566 aa, synthetic construct
MKTIIAFSCILCLIFAQKLPGSDNSMATLCLGHHAVPNGTLVKTITDDQIEVTNATELVQSSSTGRICNSPHQILDGKNCTLIDALLGDPHCDDFQNKEWDLFVERSTAYSNCYPYYVPDYASLRSLVASSGTLEFTQESFNWTGVAQDGSSYACRRGSVNSFFSRLNWLYNLNYKYPEQNVTMPNNDKFDKLYIWGVHHPGTDKDQTNLYVQASGRVIVSTKRSQQTVIPNIGSRPWVRGVSSIISIYWTIVKPGDILLINSTGNLIAPRGYFKIQSGKSSIMRSDAHIDECNSECITPNGSIPNDKPFQNVNRITYGACPRYVKQNTLKLATGMRNVPEKQTRGIFGAIAGFIENGWEGMVDGWYGFRHQNSEGTGQAADLKSTQAAINQITGKLNRVIKKTNEKFHQIEKEFSEVEGRIQDLEKYVEDTKIDLWSYNAELLVALENQHTIDLTDSEMNKLFERTRKQLRENAEDMGNGCFKIYHKCDNACIGSIRNGTYDHDVYRDEALNNRFQIKGVQLKSGYKDWILWISFAISCFLLCVVLLGFIMWACQKGNIRCNICI

SEQ ID NO: 87 - protein, 566 aa, synthetic construct
MKTIIALSYILCLVFAQKLPGNDNNMATLCLGHHAVPNGTIVKTITNDQIEVTNATELVQSTSKGEICSSPHQILDGENCTLIDALLGDPHCDDFQNKKWDLFVERSKAHSNCYPYYVPDYATLRSLVASSGNLEFTQESFNWTGVTQDGASSACKRRSSKSFFSRLNWLHNLNYKYPALNVTMPNNDNFDKLYIWGVHHPSTDKDQTSLYIQASGRVTVSTKRSQQAVIPNIGSRPRVRDIPSRISIYWTIVRPGDILLINSNGNLIAPRGYFKIRSGKSSIMRSDAPIGNCNSECITPNGSISNDKPFQNVNKITYGACPRYIKQNTLKLATGMRNIPEKQTRGIFGAKAGFIENGWEGMVNGWYGFRHQNSEGRGQAADLKSTQAAIDQINGKLNRLIGKTNEKFHQIEKEFSEIEGRIQDLERYVEDTKIDIWSYNAELLVAMENQHTIDLTDSEMSKLFERTRRQLRENAEDMGNGCLKIYHKCDNSCMESIRNGTYDHDIYRNEALNNRFQIKGVELKSEYKDWILWISFATSCFLLCAVLLGFIMWACQKGNIKCNICI

SEQ ID NO: 88 - protein, 566 aa, synthetic construct
MKAIIAFSCILCQIFAQKLPGNDNSTATLCLGHHAVSNGTLVKTITDDQVEVTNATELVQNFSMGKICKNPHRILDGANCTLIDSLLGDPHCDGFQNEKWDLFIERSKAFSNCYPYDVPDYASLRSLIASSGTLEFNNESFNWTGVTQNGGSSACKRGPNNSFFSRLNWLTHLNFKYPALEVTMPNNEQFDKLYIWGVHHPATDKDQISLYAQAAGRIIVSTKRSQQTIIPNIGSRPWIRGVSSIISIHWTIVKPGDMLLINSTGNIIAPRGYFKIQTGKSSVMRSDAPIGKCNSACITPNGSIPNDRPFQNVNKITYGACPKYVKQNTLKLATGMRNIPERQTRGIFGAIAGFIENGWEGMIDGWYGFRHHNSEGTGQAADIKSTQAAINQINGKLNRVIEKTNEKFHQIEKEFLEVEGRIQDIEKYIEDTKIDLWSYNAELLIALENQHTIDLADSEMNKLFEKTKKQLRENAEDIGNGCFKIYHKCDNTCIGSIRNGTYNHDVYRDEAVNNRFQIKGVQLKLGYKDWILWISFAMSCFLLCVALLGFIMWACQRGNIRCNICV

SEQ ID NO: 89 - protein, 465 aa, synthetic construct
MNTNQRIITIGTVCMIVGIISLLLQIGNIVSLWISHSIQTGNENHTHECNQSVITYENNTWVNQTYVNISNTNIAQVTSVTSIILAGNSSLCPVSGWAIYSKDNSIRIGSKGDIFVIREPFISCSLECRTFFLTQGALLNDKHSNGTVKDRSPYRTLMSCPIGEAPSPYNSRFESVAWSASACHDGMGWLTIGISGPDNGAVAVLKYNGIITDTIKSWRNILRTQESECVCNGSCFTVMTDGPSNGQASYKIFKEKGKIIKSVELDAPNYHYEECSCYPDTGKVVCVCRDNWHASNRPWVSFNQNLDYQIGYICSGVFGDNPRSNDGKGNCGPVLSNGANGVKGFSFRYGNGVWIGRTKSISSRSGFEMIWDPNGWTETDSSFSIKQDIIALNDWSGYSGSFVQHPELTGMNCIRPCFWVELIRGQPKENTIWTSGSSISFCGVNSETASWSWPDGADLPFTIDK

SEQ ID NO: 90 - protein, 469 aa, synthetic construct
MNTNQRIITIGTVCMIVGIISLLLQIGNIVSLWISHSIQTGWENHTEMCNQSVITYVNNTWVNRTYVNISNIKIATIQDVTSIILAGNSSLCPVSGWAVYSKDNSIRIGSKGDVFVMREPFISCSQLECRTFFLTQGALLNDKHSNGTVKDRSPHRTLMSCPVGEVPSPYNSKFESVAWSASACHDGMGWLTIGISGPDDGAVAVLKYNGIITDTIKSWRNKILRTQESECVCMNGSCFTVLTDGPSNGQASYKIFKVEKGKIIKSIELDAPNYHYEECSCYPDTGKVMCVCRDNWHASNRPWVSFNQNLDYQIGYICSGVFGDNPRSNDGKGNCGPVLSNGANGVKGFSYRYGNGVWIGRTKSINSRSGFEMIWDPNGWTETDSSFSMKQDIIALNDWSGYSGSFVQHPELTGMNCIRPCFWVELIRGQPKESTIWASGSSISFCGVNSETASWSWPDGANLPFTIDK

SEQ ID NO: 91 - protein, 469 aa, synthetic construct
MNTNQKIITIGSVCMTIGMANLILQIGNIISIWISHSIQLGNQNQIETCNQSVITYENNTWVNQTYVNISNTNFAAGQSVVSVKLAGNSSLCPVSGWAIYSKDNSVRIGSKGDVFVIREPFISCSPLECRTFFLTQGTLLNDKHSNGTIKDRSPYRTLMSCPIGEVPSPYNSRFESVAWSASACHDGINWLTIGISGPDSGAVAVLKYNGVITDTIKSWRNNILRTQESECVCINGSCFTIMTDGPSDGQASYKIFRIEKGKIVKSVEMNAPNYHYEECSCYPDSSEITCVCRDNWHASNRPWVSFDQNLDYQIGYICSGIFGDNPRPNDKTGSCGPVSSNGANGVKGFSFRYGNGVWIGRTKSISSRNGFEMIWDPNGWTGTDNNFSIKQDIVGINEWSGYSGSFVQHPELTGLDCIRPCFWVELIRGRPKENTIWTSGSSISFCGVNSDTVGWSWPDGAELPFTIDK

SEQ ID NO: 92 - protein, 469 aa, synthetic construct
MNPNQKIITIGSICMTIGIASLILQIGNIVSLWVSHSIQTRWENHTEVCNQNVITYVNNTLVNRTYVNISNTNIATIQDVTSIMLAGNSSLCPISGWAIYSKDNSIRIGSKGDIFVIREPFISCSHLECRTFFLTQGALLNDRHSNGTVKDRSPYRTLMSCPIGEAPSPYNSRFESVAWSASACHDGTSWLTIGISGPDNGAVAVLKYNDIITDTIKSWRKNILRTQESECACVNGSCFTVMTDGPSNGQASYKIFKIEKGKVVKSVELNAPNYHYEECSCYPDTGKVVCVCRDNWHGSNRPWVSFNQNLEYQIGYICSGVLGDNPRPNDRTGSCGPVFSNGANGVKGFSFKYGNGVWIGRTKSTSSRSGFEMIWDPDGWTRTDDKFSVKQDIIALTDWSGYSGSFVQHPELTGLDCMRPCFWVELIRGQPKENTIWASGSSISFCGVDSETASWSWPDGADLPFTIDK

SEQ ID NO: 93 - protein, 469 aa, synthetic construct
MNTNQRIITIGTVCMIVGIISLLLQIGNIVSLWISHSIQTGWENHTEMCNQSVITYENNTWVNQTYVNISNIKIATIQDVTSIILAGNSSLCPVSGWAIYSKDNSIRIGSKGDIFVIREPFISCSQLECRTFFLTQGALLNDKHSNGTVKDRSPYRTLMSCPIGEAPSPYNSRFESVAWSASACHDGMGWLTIGISGPDNGAVAVLKYNGIITDTIKSWRNKILRTQESECVCMNGSCFTVLTDGPSNGQASYKIFKVEKGKIIKSIELDAPNYHYEECSCYPDTGKVMCVCRDNWHASNRPWVSFNQNLDYQIGYICSGVFGDNPRSNDGKGNCGPVLSNGANGVKGFSYRYGNGVWIGRTKSINSRSGFEMIWDPNGWTETDSSFSMKQDIIALTDWSGYSGSFVQHPELTGMNCIRPCFWVELIRGQPKESTIWASGSSISFCGVNSETASWSWPDGADLPFTIDK

SEQ ID NO: 94 - protein, 469 aa, synthetic construct
MNPNQKIITIGSVCMTIGMANLILQIGNIISIWISHSIQLGNQNQIETCNQSVITYVNNTWVNRTYVNISNTNFAAGQSVVSVKLAGNSSLCPISGWAIYSKDNSVRIGSKGDVFVIREPFISCSHLECRTFFLTQGSLLNDKHSNGTIKDRSPYRTLMSCPIGEVPSPYNSRFETVAWSASACHDGINWLTIGISGPDSGAVAVLKYNDIITDTIKSWRNNILRTQESECACVNGSCFTVMTDGPSDGQASYKIFRIEKGKIVKSVEMNAPNYHYEECSCYPDSSEITCVCRDNWHGSNRPWVSFNQNLEYQIGYICSGIFGDNPRPNDKTGSCGPVSSNGANGVKGFSFKYGNGVWIGRTKSISSRNGFEMIWDPNGWTGTDNNFSIKQDIIALNDWSGYSGSFVQHPELTGLDCIRPCFWVELIRGRPKENTIWTSGSSISFCGVNSDTVGWSWPDGAELPFTIDK

SEQ ID NO: 95 - protein, 469 aa, synthetic construct
MNTNQKIITIGSICMTIGIASLILQIGNIISIWVSHSIQTRWENHTEVCNQNVITYVNNTWVNQTYVNISNTNIATIQDVTPIILAGNSPLCPVSGWAVYSKDNSIRIGSKGDIFVMREPFISCSPLECRTFFLTQGALLNDRHSNGTVKDRSPHRTLMSCPVGEVPSPYNSKFESVAWSASACHDGTSWLTIGISGPDDGAVAVLKYNGVITDTIKSWRKNILRTQESECVCINGSCFTIMTDGPSNGQASYKIFKIEKGKVVKSVELNAPNYHYEECSCYPDTGKVVCVCRDNWHASNRPWVSFDQNLDYQIGYICSGVLGDNPRPNDRTGSCGPVFSNGANGVKGFSFRYGNGVWIGRTKSTSSRSGFEMIWDPDGWTRTDDKFSVKQDIVGINEWSGYSGSFIQHPELTGLDCMRPCFWVELIRGQPKENTIWASGSSISFCGVDSETASWSWPDGANLPFTIDK

SEQ ID NO: 96 - protein, 466 aa, synthetic construct
MNPNQKIITIGSVSLIIATICFLMQIAILVTTVTLHFKQHDCNSPPNNQVMLCEPPTIIERNTEIVYLTNTTIEKEICPKLAEYRNWSKPQCNITGFAPFSKDNSIRLSAGGDIWVTREPYVSCDPDKCYQFALGQGTTLNNGHSNDTVHDRTPYRTLLMNELGVPFHLGTRQVCIAWSSSSCHDGKAWLHVCITGDDENATASFIYNGRLVDSIGSWSKNILRTQESECVCINGTCTVVMTDGSASGKADTKILFIEEGKIIHISTLSGSAQHVEECSCYPRYPGVRCVCRDNWKGSNRPIVDINVKDYSIVSSYVCSGLVGDTPRKNDSSSSCLDPNNEEGGHGVKGWAFDDGNDVWMGRTISEELRLGYETFKVIGWSKPNSKLQTNRQVIVERGNRSGYSGIFSVEGKSCINRCFYVELIRGRKEETKVWWTSNSIVVFCGTSGTYGTGSWPDGADINLMPI

SEQ ID NO: 97 - protein, 469 aa, synthetic construct
MNPNQKIITIGSVSLTIATICFLMQIAILVTTVTLHFKQHDYNSPPNNQAMLCEPTIIERNTTEIVYLTNTTIEKEICPKLAEYRSWSKPQCNITGFAPFSRDNSIRLSAGGDIWVTREPYVSCDHDKCYQFALGQGTTLNNGHSNNTVHDRTPYRTLLMNELGVPFHLGTRQVCMAWSSSSCHDGRAWLHVCITGNDNNATASFIYDGRLVDSIGSWSKKILRTQESECVCINGICTVVMTDGSASEKADTKILFVEEGKIVHISTLSGSAQHVEECSCYPRFPGVRCVCRDNWKGSNRPIVDINVKNYSIVSSYVCSGLVGDTPRKSDSVSSSYCLDPNNEKGGHGVKGWAFDDGNDVWMGRTINETLRLGYETFKVIEGWSKANSKLQTNRQVIVEKGDRSGYSGIFSIEGKNCINRCFYVELIRGRKQETEVWWTSNSIVVFCGTSGTYGAGSWPDGADISLMPI

SEQ ID NO: 98 - protein, 469 aa, synthetic construct
MNPNQKIITIGSISLTLAAMCFLMQIAILVTNVTLHFKQHNCDSSPNNHVMFCEPTIIERNITEIVYLTNITIEKEKCPKLAEYRNWSKPQCKITGFAPFSKDNSIRLSAGGAIWVTREPYVSCDPDKCYQFALGQGTTLNNRHSNDTIHDRTPYRTLLMSELGVPFHLGTRQVCIAWSSSSCYDGKAWLHVCITGDDKNATASFIYNGRLVDSIGSWSKNILRTQESECVCINGTCAVVMTDGSASGKADTKILFIEEGKIVHISPLLGSAQHVEECSCYPRYPGVRCVCRDNWRGSNRPIVDINVKDYSIVSSYVCSGLVGDTPRKDDKSSSSNCLNPNNEEGGHGVKGWAFDDGDDVWMGRTISEKLRSGYETFKVIEGWSKPNSKLQINRQVIVDRGNRSGYSGIFSVEGKSCINRCFYVELIRGRRQETRVWWTSNSIIVFCGTSGTYGSGSWPDGADINFMPI

SEQ ID NO: 99 - protein, 469 aa, synthetic construct
MNPNQRIITIGSVSLIIATICFLMQVAILVTTVTLHFKQHDCNSSPNNQVMLCEPTIIERNKTEIVYLTNTTVEKEICPKPAEYRNWSKPQCNIAGFAPFSKDNSVRLSAGGDIWITREPYVSCDLDKCYQFALGHGTTLNNGHSNDTVHDRTPYRTLLMNELGIPFHLGTKQVCIAWSSSSCHDGKAWLHVCVTGHDENATASIIYNGRLVDSIGSWSQNILRTQESECVCINGTCTVVMTDGSASGRADTKILFIEEGKIIHISTLSGSAKHVEECSCYPRYPGVRCICRDNWKGSNRPVVDINVKDYSTVSSYVCSGLVGDTPRKNDSFSSSHCLDPNNEEGGHGVKGWAFDDGNDLWMGRTISEKSRLGYETFKVIKGWSKPNSKLQTNRQVIVGRGNRSGYSGIFSVEGKNCINRCFYVELIRGRKEETKVWWTSNSILVFCGTSGTYGTGSWPDGADINLMPI

SEQ ID NO: 100 - protein, 469 aa, synthetic construct
MNPNQKIITIGSVSLIIATICFLMQIAILVTTVTLHFKQHDYNSPPNNQVMLCEPTIIERNKTEIVYLTNTTIEKEICPKLAEYRNWSKPQCNITGFAPFSKDNSIRLSAGGDIWVTREPYVSCDPDKCYQFALGQGTTLNNGHSNDTVHDRTPYRTLLMNELGVPFHLGTRQVCIAWSSSSCHDGKAWLHVCITGDDKNATASFIYNGRLVDSIGSWSKNILRTQESECVCINGTCTVVMTDGSASGKADTKILFIEEGKIIHISTLSGSAQHVEECSCYPRYPGVRCVCRDNWKGSNRPIVDINVKDYSIVSSYVCSGLVGDTPRKNDSFSSSHCLDPNNEEGGHGVKGWAFDDGNDVWMGRTINETLRLGYETFKVIEGWSKPNSKLQTNRQVIVEKGDRSGYSGIFSVEGKSCINRCFYVELIRGRKEETKVWWTSNSIVVFCGTSGTYGTGSWPDGADINLMPI

SEQ ID NO: 101 - protein, 469 aa, synthetic construct
MNPNQRIITIGSVSLTIATICFLMQVAILVTTVTLHFKQHDCNSSPNNQVMFCEPTIIERNTTEIVYLTNTTVEKEICPKPAEYRNWSKPQCKITGFAPFSRDNSIRLSAGGAIWVTREPYVSCDLDKCYQFALGQGTTLNNRHSNDTIHDRTPYRTLLMNELGIPFHLGTKQVCIAWSSSSCYDGKAWLHVCITGNDNNATASFIYDGRLVDSIGSWSKKILRTQESECVCINGICTVVMTDGSASGRADTKILFVEEGKIVHISTLSGSAKHVEECSCYPRYPGVRCICRDNWKGSNRPVVDINVKNYSIVSSYVCSGLVGDTPRKSDSVSSSYCLDPNNEKGGHGVKGWAFDDGDDVWMGRTISEKSRLGYETFKVIKGWSKPNSKLQINRQVIVDRGNRSGYSGIFSVEGKNCINRCFYVELIRGRKQETEVWWTSNSIIVFCGTSGTYGSGSWPDGADINFMPI

SEQ ID NO: 102 - protein, 469 aa, synthetic construct
MNSNQKIITIGSISLTLAAMCFLMQIAILVTNVTLHFNQCECHYPPNNQVILCEPTIIERNITEIVYLTNITIEKEKCPKLAEYRSWSKPQCNIAGFAPFSKDNSVRLSAGGDIWITREPYVSCDHDKCYQFALGHGTTLNNGHSNNTVHDRTPYRTLLMSELGVPFHLGTRQVCMAWSSSSCHDGKAWLHVCVTGHDENATASIIYNGRLVDSIGSWSQNILRTQESECVCINGTCAVVMTDGSASGKADTRILFIEEGKIVHISPLLGSAQHVEECSCYPRFPGVRCVCRDNWRGSNRPVVDINVKDYSTVSSYVCSGLVGDTPRKDDKSSSSNCLNPNNEEGGHGVKGWAFDDGNDLWMGRTISEKLRSGYETFKVIEGWSKANSKLQTNRQVIVGRGNRSGYSGIFSIEGKNCINRCFYVELIRGRRQETRVWWTSNSILVFCGTSGTYGAGSWPDGADISLMPI

SEQ ID NO: 103 - DNA, 1680 bp, synthetic construct
  source 1..1680: mol_type = other DNA
atgaaggtgaagctgctggtgctgctgtgcacctttacagccacctacgccgacacaatctgtatcggctatcacgccaacaattccacagacaccgtggataccgtgctggagaagaacgtgacagtgacccactccgtgaacctgctggaggattctcacaatggcaagctgtgcctgctgaagggcatcgccccactgcagctgggcaactgctctgtggccggctggatcctgggcaatcccgagtgtgagctgctgatcagcaaggagtcttggagctacatcgtggagacaccaaaccccgagaatggcacctgctaccccggctttgccgactatgaggagctgagggagcagctgagcagcgtgagcagcttcgagcggttcgagatcttccctaaggagtcctcttggccaaaccacacagtgaccaagggcgtgtccgcctcttgtagccacaacggcagctccttctaccggaatctgctgtggctgaccggcaagaacggcctgtaccctaatctgtccaagtcttatgccaacaataaggagaaggaggtgctggtgctgtggggagtgcaccacccacctaacatcggcgatcagctgtaccacacagagaatgcctatgtgagcgtggtgtctagccactactcccggtttacccccgagatcgccaagaggcctaaggtgcgcgaccaggagggcagaatcaactactattggacactgctggagcccggcgataccatcatcttcgaggccaacggcaatctgatcgcccctagatatgcctttgccctgtccaggggattcggaagcggaatcatctccaatgcccctatggacgagtgcgatgccaagtgtcagacaccacagggcgccatcaactcctctctgccatttcagaatgtgcaccccgtgacaatcggcgagtgtcctaagtacgtgcggagcgccaagctgagaatggtgaccggcctgcggaacatcccaagcatccagtccagaggcctgtttggagcaatcgcaggcttcatcgagggaggatggaccggaatggtggacggctggtacggctatcaccaccagaatgagcagggctctggctatgccgccgatcagaagagcacacagaacgccatcaatggcatcaccaacaaggtcaattccgtgatcgagaagatgaacacacagtttaccgccgtgggcaaggagttcaataagctggagcggagaatggagaacctgaataagaaggtggacgatggctttctggacatctggacatacaacgccgagctgctggtgctgctggagaatgagaggaccctggacttccacgattccaacgtgaagaatctgtatgagaaggtgaagtctcagctgaagaacaatgccaaggagatcggcaacggctgcttcgagttttaccacaagtgcaacgacgagtgtatggagagcgtgaagaatggcacatacgattatcccaagtattctgaggagagcaagctgaatagggagaagatcgatggcgtgaagctggagagcatgggcgtgtaccagatcctggccatctattccaccgtggcaagctccctggtgctgctggtgtctctgggcgccatcagcttctggatgtgctccaacggctctctgcagtgccgcatctgtatc

SEQ ID NO: 104 - DNA, 1695 bp, synthetic construct
  source 1..1695: mol_type = other DNA
atgaaggtgaagctgctggtgctgctgtgcaccttcacagccacctacgccgacacaatctgtatcggctatcacgccaacaatagcacagacaccgtggataccgtgctggagaagaacgtgacagtgacccactctgtgaatctgctggagaacagccacaatggcaagctgtgcctgctgaagggcatcgccccactgcagctgggaaactgcagcgtggcaggatggatcctgggaaatccagagtgtgagctgctgatctccaaggagtcttggagctacatcgtggagaagccaaaccccgagaatggcacatgctaccccggccacttcgccgactatgaggagctgagggagcagctgagctccgtgtctagcctggagcggttcgagatctttcctaaggagtcctcttggccaaaccacacagtgaccggcgtgtccgcctcttgtagccacaacggcgagagctccttttacaggaatctgctgtggctgaccggcaagaacggcctgtaccctaatctgtccaagtcttatgccaacaataaggagaaggaggtgctggtgctgtggggagtgcaccacccacctaacatcggcgatcagcgcgccctgtaccacacagagaatgcctacgtgagcgtggtgtctagccactactctcggaagttcacccccgagatcgccaagaggcctaaggtgcgcgaccaggagggcagaatcaactactattggacactgctggagcccggcgataccatcatctttgaggccaacggcaatctgatcgcccctagatatgccttcgccctgagcaggggatttggatccggcatcatcaactctaatgcccctatggacaagtgcgatgccaagtgtcagaccccacagggcgccatcaactcctctctgccattccagaatgtgcaccccgtgacaatcggcgagtgtcctaagtacgtgcggagcgccaagctgagaatggtgaccggcctgcggaacatcccaagcgtgcagtccagaggactgttcggagcaatcgcaggctttatcgagggaggctggacaggcatgatcgacggctggtacggctatcaccaccagaatgagcagggcagcggctatgccgccgatcagaagtccacacagaacgccatcaatggcatcaccaacaaggtcaattccgtgatcgagaagatgaacatccagttcaccgccgtgggcaaggagtttaataagctggagcggagaatggagaacctgaataagaaggtggacgatggcttcatcgatatctggacatacaacgccgagctgctggtgctgctggagaatgagcggaccctggactttcacgattctaacgtgaagaatctgtatgagaaggtgaagagccagctgaagaacaatgccaaggaagtgggcaacggctgcttcgagttttaccacaagtgcaacgacgagtgtatggagtccgtgaagaatgagacctacgattatcccaagtattctgaggagagcaagctgaatagggagaagatcgacggcgtgaagctggattccatgggcgtgtaccagatcctgacaatctattctaccgtggccagctccctggtgctgctggtgagcctgggagccatctccttttggatgtgctccaacggctctctgcagtgcaagatctgtatc

SEQ ID NO: 105 - DNA, 1698 bp, synthetic construct
  source 1..1698: mol_type = other DNA
atgaaggccatcctggtggtgctgctgtacacctttgccacagccaacgccgacaccctgtgcatcggctatcacgccaacaatagcaccgacacagtggatacagtgctggagaagaatgtgaccgtgacacactccgtgaacctgctggaggataagcacaatggcaagctgtgcaagctgaggggagtggcacctctgcacctgggcaagtgcaacatcgccggctggatcctgggcaatccagagtgtgagtccctgtctaccgccagctcctggtcctacatcgtggagacctctagctccgacaacggcacatgctaccctggcgactttatcgattatgaggagctgagggagcagctgtctagcgtgagcagcttcgagcggttcgagatcttccccaagaccagctcctggcctaaccacgatagcaataagggagtgacagcagcatgtccacacgcaggcgccaagtccttctacaagaacctgatctggctggtgaagaagggcaattcttaccccaagctgagcaagtcctatatcaacgacaagggcaaggaggtgctggtgctgtggggcatccaccacccatctaccagcgccgaccagcagtccctgtaccagaatgccgatgcctacgtgttcgtgggcacatcccggtactctaagaagttcaagccagagatcgccatccggcccaaggtgagagaccaggagggcagaatgaactactattggaccctggtggagccaggcgataagatcacctttgaggcaacaggaaacctggtggtgcccagatatgccttcgccatggagagaaatgccggctccggcatcatcatctctgacacccccgtgcacgattgcaacaccacatgtcagacccctaagggcgccatcaacacatctctgcctttccagaatatccacccaatcacaatcggcaagtgccccaagtacgtgaagagcaccaagctgaggctggccacaggcctgcgcaatgtgcctagcatccagtccaggggactgtttggagcaatcgcaggcttcatcgagggaggatggaccggaatggtggatggatggtacggctatcaccaccagaacgagcagggctctggctatgccgccgacctgaagagcacccagaatgccatcgataagatcacaaacaaggtcaatagcgtgatcgagaagatgaacacccagtttacagccgtgggcaaggagttcaatcacctggagaagcgcatcgagaacctgaataagaaggtggacgatggctttctggacatctggacctacaacgccgagctgctggtgctgctggagaatgagaggacactggactaccacgattccaacgtgaagaatctgtatgagaaggtgcgctctcagctgaagaacaatgccaaggagatcggcaacggctgcttcgagttttaccacaagtgcgacaacacctgtatggagtctgtgaagaatggcacatacgattatcccaagtatagcgaggaggccaagctgaatcgggaggagatcgatggcgtgaagctggagagcaccagaatctaccagatcctggccatctattccacagtggcctctagcctggtgctggtggtgagcctgggagcaatctccttctggatgtgctctaacggcagcctgcagtgcaggatctgtatc

SEQ ID NO: 106 - DNA, 1689 bp, synthetic construct
  source 1..1689: mol_type = other DNA
atgaaggtgaagctgctggtgctgctgtgcaccttcacagccacctacgccgacacaatctgtatcggctatcacgccaacaatagcacagacaccgtggataccatcctggagaagaacgtgacagtgacccactctgtgaatctgctggagaacagccacaatggcaagctgtgcctgctgaagggcatcgccccactgcagctgggaaactgctccgtggcaggatggatcctgggaaatccagagtgtgagctgctgatctctaaggagtcttggagctacatcgtggagaagccaaaccccgagaatggcacatgctaccccggcgacttcatcaactatgaggagctgagggagcagctgagctccgtgtctagcctggagcggttcgagatctttcctaaggagtcctcttggccaaatcacacagtgaccggcgtgtccgcctcttgtagccacaacggcgagagctccttttaccggaatctgctgtggctgaccggcaagaacggcctgtaccctaatctgtccaagtcttatgccaacaataaggagaaggaggtgctggtgctgtggggagtgcaccacccacctaacatcggcgatcagagagccctgtaccacacagagaatgcctacgtgagcgtggtgtctagccactactctaggaagttcacccccgagatcgccaagaggcctaaggtgcgcgaccaggagggacgcatcaactactattggacactgctggagcccggcgataccatcatctttgaggccaacggcaatctgatcgcccctagatatgccttcgccctgagcaggggatttggatccggcatcatcaactctaatgcccctatggacaagtgcgatgccaagtgtcagaccccacagggcgccatcaactcctctctgccattccagaatgtgcaccccgtgacaatcggcgagtgtcctaagtacgtgcggagcgccaagctgagaatggtgaccggcctgcggaacatcccaagcatccagtccagaggactgctgggagcaatcgcaggctttatcgagggaggctggacaggcatgatcgacggctggtacggctatcaccaccagaacgcacagggaagcggatatgcagcagaccagaagtccacacagaatgccatcgatgagatcaccaacaaggtcaattccgtgatcgagaagatgaacatccagttcaccgccgtgggcaaggagtttaataagctggagcggagaatggagaacctgaataagaaggtggacgatggcttcatcgatatctggacatacaacgccgagctgctgatcctgctggagaatgagcggaccctggactttcacgatagcaacgtgaagaatctgtatgagaaggtgagaaaccagctgaagaacaatgccaaggaagtgggcaatggctgcttcgagttttaccacaagtgcaacgacgagtgtatggagtccgtgaagaatggcatctacgattatcccaagtattctgaggagagcaagctgaacagggagaagatcgatggcgtgaagctggagtctatgggcgtgtaccagatcctggccatctatagcaccgtggcaagctccctggtgctgctggtgagcctgggagccatctccttttggatgtgctccggccactcttgcagaatctgtatc

SEQ ID NO: 107 - DNA, 1698 bp, synthetic construct
  source 1..1698: mol_type = other DNA
atggaggccatcctggtggtgctgctgtacacctttaccacagccaacgccgacacactgtgcgtgggctatcacgccaacaatagcaccgacacagtggataccgtgctggagcggaatgtgaccgtgacacactccgtgaacctgctggaggattctcacaatggcaagctgtgcagactgaagggcatcgcccctctgcagctgggcaagtgcaacatcgccggctggctgctgggcaatccagagtgtgagtccctgttttctaaggagagctggtcctacatcgtggagacccccaaccctgagaatggcacatgctaccctggccacttcgccgactatgaggagctgagggagcagctgaacagcgtgagcagcttcgagcggttcgagatgttcccaaagacctctagctggcccgaccacgattccaataagggagtgaccacagcatgtccacacgcaggagcaaagggcttttacaagaacctgatctggctgacaaagaagggcaatagctaccccaagctgtctcagagctatatcaacgataagggcaaggagatcctggtgctgtggggcatccaccacccaagcaccacagccgaccagcagtccctgtaccagaatgccgatacctacgtgttcgtgggcacaagcaagtactccaagaagttcaagcctgagatcgccacccggccaaaggtgagagaccaggagcggagaatgaactactattggaccctgatcgagccaggcgataagatcacatttgaggccaacggcaatctggtggtgccccggtatgccttcaccatggagagaaacgccggctccggcatcatcatctctgacacaagcgtgcacgattgcaataccacatgtcagacccccgagggcgccatcaacacatccctgcccttccacaatatccaccctatcaccatcggcgagtgccccaagtacgtgaggtctaccaagctgcgcatggtgacaggcctgaggaacgtgccttccgtgcagtctaggggactgtttggagcaatcgcaggcttcatcgagggaggatggaccggaatgatggacggatggtacggctatcaccaccagaatggccagggctctggctatgccgccgatcagaagagcacccagaacgccatcaatggcatcacaaacaaggtcaattccgtgatcgacaagatgaacacccagtttacagccgtgggcaaggagttcaataagctggagaagaggatggagaacctgaataagaaggtggacgatggctttatcgacgtgtggacctacaacgccgagctgctgatcctgctggagaatgagcgcacactggactaccacgattccaaggtgaagaacctgtatgagaaggtgaagtctcagctgaagaacaatgccaaggagaccggcaatggctgcttcgagttttaccacaagtgcaacaatgagtgtatggagtctgtgaagaacggcacatacgactatcctcggtatagcgaggaggccaagctgaatagagagaagatcgatggcgtgaagctggagagcacccgggtgtaccagatcctggccatctattccacagccgcctcctctctggtgctggtggtgtctctgggcgagatcagcttctggatgtgcagcaacggctccctgaggtgccgcatctgtatc

SEQ ID NO: 108 - DNA, 1692 bp, synthetic construct
  source 1..1692: mol_type = other DNA
atgaagaccatcatcgccctgtcttatatcctgtgcctggtgttcgcccagaagctgccaggcaatgacaacaatagcaccgccacactgtgcctgggacaccacgccgtgcccaacggcacaatcgtgaagaccatcacaaatgatcagatcgaggtgaccaacgccacagagctggtgcagagctccaccggcgagatctgcgacagccctcaccagatcctggatggcgagaactgtacactgatcgacgcactgctgggcgacccacagtgcgatggcttccagaataagaagtgggatctgtttgtggagcggtctaaggcctacagcaactgttacccctatgacgtgcctgattatgcctccctgagatctctggtggcctctagcggcaccctggagttcaacaatgagtcttttaattggaccggcgtgacacagaacggcacatcctctgcctgcatccggagatctaatagcttctttagcaggctgaattggctgactcacctgaacttcaagtaccccgccctgaacgtgacaatgcctaacaatgagcagtttgacaagctgtatatctggggagtgcaccacccaggcaccgacaaggatcagatcttcctgtacgcacagtccggaaggatcaccgtgtccacaaagagatctcagcaggccgtgatccctaatatcggatctaggccaaggaggatcccaagccgcatctccatctattggaccatcgtgaagcctggcgacatcctgctgatcaacagcacaggcaatctgatcgccccaaggggctacttcaagatccgctctggcaagagctccatcatgcggagcgacgccccaatcggcaagtgctccgagtgtatcacccccaatggctctatccctaacgataagccatttcagaacgtgaatcggatcacatacggcgcctgtcccagatatgtgaagcagaccctgaagctggccacaggcatgaggaacgtgcctgagaagcagaccaggggaatcttcggagcaatcgcaggctttatcgagaatggctgggagggcatggtggacggctggtacggctttcggcaccagaacagcgagggaagaggacaggcagcagacctgaagtccacccaggcagccatcgatcagatcaacggcaagctgaataggctgatcggcaagacaaatgagaagttccaccagatcgagaaggagttttccgaggtggagggccgcatccaggatctggagaagtacgtggaggacaccaagatcgatctgtggagctataatgccgagctgctggtggccctggagaaccagcacaccatcgacctgacagattccgagatgaacaagctgttcgagaagaccaagaagcagctgcgggagaatgccgaggacatgggcaacggctgctttaagatctatcacaagtgcgataatgcctgtatcggctccatcagaaacggcacatacgaccacgacgtgtacagggacgaggccctgaacaatcgcttccagatcaagggcgtggagctgaagtctggctacaaggattggatcctgtggatcagcttcgccatctcctgctttctgctgtgcgtggccctgctgggctttatcatgtgggcctgccagaagggcaacatccggtgtaatatcagatgcaacatctgtatc

SEQ ID NO: 109 - DNA, 1698 bp, synthetic construct
  source 1..1698: mol_type = other DNA
atgaagaccatcatcgccctgtctcacatcctgtgcctggtgttcgcccagaagatcccaggcaacgacaatagcaccgccacactgtgcatcggacaccacgccgtgcccaacggcatgatcgtgaagaccatcacaaatgatagaatcgaggtgaccaacgccacagagctggtgcagaatagctccatcggcgagatctgcgacagcccccaccagatcctggatggcgagaattgtacactgatcgacgcactgctgggcgaccctcactgcgatggctttcagaacgagaagtgggatctgttcgtggagagatctaaggcctttagcaattgttacccctatgacgtgcctgattacgcctccctgaggtctctggtggcctctagcggcaccctggagttcaagaatgagtcctttaactggaccggcgtggcccagaacggcacatcctctgcctgcatccggagaagctcctctagcttcttttctaggctgaattggctgacccacctgaactacacatatccagccctgaatgtgacaatgcccaacaaggagcagttcgacaagctgtacatctggggagtgcaccacccaggcaccgacaaggatcagatctttctgtatgcccagtcctctggccgcatcaccgtgtccacaaagcggtctcagcagacagccatcccaaatatcggaagcaggccaaggatcagggacatccctagccgcatctccatctactggaccatcgtgaagcccggcgacgtgctggtcatcaactccaatggcaacctgatcgcccctcgcggctatttcaagatccagtccggcaagagctccatcatgcggtctgacgcccccatcggcaagtgcaagagcgagtgtatcacacctaatggctctatcagcaacgataagccatttcagaatgtgaacaagatcacctacggcgcctgtcctagatatgtgaagcactccaccctgaagctggccacaggcatgagaaacgtgccagagaagcagaccaggggactgttcggagcaatcgcaggctttatcgagaacggctgggagggcatgaaggacggctggtacggcttcagacaccagaatagcgagggaaggggacaggcagcagatctgaagtccacacaggccgccatcaatcagatcaacggcaagctgaaccgcctgatcggcaagaccaatgagaagttccaccagatcgagaaggagtttagcgaggtggagggaagggtgcaggacctggagaagtacgtggaggactctaagatcgatctgtggagctataacgccggcctgctggtggccctggagaatcagcacaccatcgacctgacagattccgagatgaacaagctgttcgagagaaccaggaagcagctgagagagaatgccgaggacatgggcaacggctgttttacaatctaccacaagtgcgataacgcctgtatcggctctatcaggaatggcacctacgaccacaacgtgtatcgcgatgaggccctgaataagcggttccagatcaagggcatcgagctgaagtccggctataaggattggatcctgtggatcagcttcgccatctcctgctttctgctgtgcgtggccctgctgggctttatcatgtgggcctgccagaagggcaatatccggtgcaacatctgtatc

SEQ ID NO: 110 - DNA, 1698 bp, synthetic construct
  source 1..1698: mol_type = other DNA
atgaagaccatcatcgccctgtcttatatcctgtgcctggtgttcgcccagaagctgcctggcaacgacaatagcaccgccacactgtgcctgggacaccacgccgtgccaaatggcacaatcgtgaagaccatcacaaacgatcagatcgaggtgaccaatgccacagagctggtgcagaacagctccatcggcgagatctgcgactccccacaccagatcctggatggcgagaactgtaccctgatcgacgcactgctgggcgacccacagtgcgatggcttccagaataagaagtgggatctgtttgtggagagatctaaggcctacagcaactgttacccctatgacgtgcctgattatgcctccctgaggtctctggtggcctctagcggcaccctggagttcaacaatgagagctttaattggaccggcgtgacacagaacggcacatcctctgcctgcatccggagaagcaacaattccttcttttctagactgaactggctgactcacctgaatttcaagtaccctgccctgaatgtgacaatgccaaacaatgagcagtttgacaagctgtatatctggggagtgcaccaccctggcaccgacaaggatcagatcttcctgtacgcacagagctccggaaggatcaccgtgtccacaaagcggtctcagcaggccgtgatccctaacatcggaagcaggccaaggatcagggacatcccaagccgcatctccatctattggaccatcgtgaagccaggcgatatcctgctgatcaactccacaggcaatctgatcgcccccagaggctacttcaagatcaggtctggcaagtctagcatcatgcggagcgacgcccccatcggcaagtgcaagtccgagtgtatcacccctaacggctctatcccaaatgataagccctttcagaacgtgaatagaatcacatacggcgcctgtcccagatatgtgaagcagaataccctgaagctggccacaggcatgcgcaacgtgcctgagaagcagaccaggggaatcttcggagcaatcgcaggctttatcgagaatggctgggagggcatggtggacggctggtacggctttagacaccagaacagcgagggaaggggacaggcagcagacctgaagtccacccaggccgccatcgatcagatcaacggcaagctgaatcgcctgatcggcaagacaaacgagaagttccaccagatcgagaaggagttttccgaggtggagggccggatccaggatctggagaagtacgtggaggacaccaagatcgatctgtggtcctataatgccgagctgctggtggccctggagaaccagcacaccatcgacctgacagattctgagatgaacaagctgttcgagaagaccaagaagcagctgagagagaacgccgaggacatgggcaatggctgttttaagatctatcacaagtgcgataatgcctgtatcggctctatcaggaacggcacatacgaccacgacgtgtaccgcgacgaggccctgaacaatcggttccagatcaagggcgtggagctgaagagcggctacaaggattggatcctgtggatcagcttcgccatctcctgctttctgctgtgcgtggccctgctgggctttatcatgtgggcctgccagaagggcaacatcaggtgcaatatctgtatc

SEQ ID NO: 111 - DNA, 1698 bp, synthetic construct
  source 1..1698: mol_type = other DNA
atgaagaccatcatcgccctgtcccacatcctgtgcctggtgttcgcccagaagatccccggcaacgacaattccaccgcaatgctgtgcctgggacaccacgccgtgtctaatggcacaatcgtgaagaccatcacaaatgatcgcatcgaggtgaccaacgccacagagctggtgcagagctcctctaccggcgagatctgcgacagccctcaccggatcctggatggcaagaactgtacactgatcgacgcactgctgggcgacccacactgcgatggcttccagaataaggagtgggatctgtttgtggagcggagcaaggcccactctaactgttacccctatgacgtgcctgattacgtgagcctgcggtccctggtggcaagctccggcaccctggagttcaagaatgagtcttttaactggaccggcgtggcccagaacggcacatctagcgcctgcatccggagatcctctagctccttctttagcagactgaattggctgacccacctgaactacacatatccagccctgaatgtgacaatgcccaacaatgagaagttcgacaagctgtacatctggggagtgcaccacccaggcaccgacaacgatcagatctttctgtatgcacaggccagcggaaggatcaccgtgagcacaaagcggtcccagcagacagtgatcccaaatatcggatccaggccacgcgtgcggaacatcccttctaggatcagcatctactggaccatcgtgaagcccggcgacgtgctggtcatcaacagcaatggcaacctgatcgcccctcgcggctatttcaagatccagagcggcaagtctagcatcatgcggtccgacgcccctatcggcaagtgcaattccgagtgtatcacacctaatggctccatctctaacgataagccatttcagaatgtgaacaagatcacctacggcgcctgtccaagatatgtgaagcactccaccctgaagctggccacaggcatgaggaacgtgcccgagagacagacaaggggcatcttcggcgccatcgccgtgtttatcgagaatggctgggagggcatgatggacggctggtacggcttccgccaccagaactctgagggcaccggacaggcagcagatctgaagagcacacaggccgccatcaatcagatcaacggcaagctgaatagactgatcgagaagaccaacgagaagttccaccagatcgagaaggagttttctgaggtggagggcagggtgcaggacctggagaagtacgtggaggactccaagatcgatctgtggtcttataatgcaggactgctggtggccctggagaaccagcacaccatccacctgacagatagcgagatgaacaagctgttcgagagaaccaagaagcagctgagggagaatgccgaggacatgggcaacggctgttttacaatctaccacaagtgcgataatgcctgtatcgagtccatcagaaatggcacctacgaccacaacgtgtatagagatgaggccctgaacaagaggttccagatcaagggcatcgagctgaagtctggctataaggactgggtgctgtggatctctttcgccatcagctgctttctgctgtgcgtggtgctgctgggctttatcatgtgggcctgccagcgcggcaatatccggtgcaacatctgtatc

SEQ ID NO: 112 - DNA, 1698 bp, synthetic construct
  source 1..1698: mol_type = other DNA
atgagaaccatcatcgccctgagctatatcttctgtctggtgtttgcccagaagctgccagagaacgacaattccaccgccacactgtgcatcggacaccacgccgtgcccaacggcacactggtgaagaccatcacaaacgatcaggtggaggtgaccaatgccacagagctggtgcagagctcctctaccggaggaatctgcgactccccacaccagatcctggatggcaagaattgtacactggtggacgcactgctgggcgaccctcactgcgatggcttccagaacgagaagtgggatctgttcgtggagagatctaaggcctttagcaattgttacccatatgacgtgcaggattacgcctctctgaggagcctgatcgcaagctccggcaccctggagttcaacaatgagagctttaactgggccggcgtgacccagaatggcacatctagcgcctgcaagcggagatctaactcctctttctttagcaggctgaattggctgacccacctgaagttcaagtacccagccctgaacgtgacaatgcccaataaggagcagtttgacaagctgtatatctggggagtgcaccacccatccaccgactctgatcagatctccctgtacgcccaggcctctggcagagtgaccgtgtccacaaagaggtctcagcagacagccatcccaaacatcggctccaggccccgcatccggaatatccctagccgcgtgtccatctattggaccatcgtgaagcccggcgatatcctgctgatcaactgtacaggcaatctgatcgcccctcgcggctacttcaagatccggaacggcaagagctccatcatgcggtccaacgcccctatcggcaagtgcaatagcgcctgtatcaccccaaacggctccatccccaatggcaagccttttcagaacgtgaatagagtgacatacggcgcctgccctagatatgtgaagcagtctaccctgaagctggccacaggcatgcgcaacgtgccagagaagcagaccaggggactgttcggagcaatcgcaggctttatcgagaacggctgggagggcatgaaggacggctggtacggcttccggcaccagaattccgagggaatcggacaggcagcagacctgaagtctacacaggccgccgtggatcagatcaacggcaagctgaatcgcgtgatcgagaagaccaacgagaagttccaccagatcgagaaggagtttacagaggtggagggccggatccaggacatcgagaagtacgtggaggacaccaaggtggatctgtggtcttataacgccgagctgctggtgagcctggagaatcagcacaccatcgacatgacagatagcgagatgaataagctgttcgagagaaccaggaagcagctgagagagaacgccgaggatatcggcaatggctgttttaagatctatcacaagtgcaacaatgcctgtatcggctctatcaggaacgagacatacgaccacaacgtgtaccgcgatgaggccctgaacaatcggttccacatcaagggcgtggagctgaagagcggctacaagaactggatcctgtggatcagcttcgccacctcctgctttctgctgtgcatcgtgctgctgggctttatcatgtgggcctgccagaagggcaacatcaagtgcaatatctgtatc

SEQ ID NO: 113 - DNA, 1713 bp, synthetic construct
  source 1..1713: mol_type = other DNA
atgaagaccatcatcgccttctcttgcatcctgtgcctgatctttgcccagaagctgcccggctccgacaacaattctatggccaccctgtgcctgggacaccacgcagtgcctaatggcacactggtgaagaccatcacagacgatcagatcgaggtgaccaacgccacagagctggtgcagagctcctctaccggcaggatctgtaattcccctggccaccagatcctggatggcaagaactgcacactgatcgacgccctgctgggcgatccacactgtgacgatttccagaataagtgggacctgtttgtggagcggagcaccgcctactccggcaactgctatccctactatgtgcctgattacgccagcctgagagagcagctgtccctggtggccagctccggcaccctggagttcacacaggagtcttttaattggaccggagtggcacaggacggctctagctacgcctgtcggagaggcagcgtgaacaattctttctttagccgcctgaactggctgtatctgaattacaagtatcccgccctgaacgtgacaatgcctaacaatgacaagttcgataagctgtacatctggggagtgcaccacccaggaaccgacaaggatcagacaaatctgtatgtgcaggccagcggcagggtggtgtctaccaagcgcagccagcagacagtgatccctaacatcggctcccggccatgggtgagaggcgtgtcctctatcatctctatctactggaccatcgtgaagcctggcgacatcctgctgatcaacagcacaggcaatctgatcgccccaaggggctatttcaagatccagtccggcaagagctccggcatcatgcgctctgacgccccaatcgattgcaacagcgagtgtatcacccccaacggctccatcccaaatgataagccctttcagaacgtgaataggatcacatacggcgcctgcccacgctatgtgaagcagaataccctgaagctggccacaggcatgcggaacgtgcccgagaagcagaccagaggcatcttcggcgccatcgccggctttatcgagaatggctgggagggcatggtggacggctggtacggcttccggcaccagaactctgagggaaccggacaggcagcagatctgaagagcacccaggcagccatcaaccagatcacaggcaagctgaatagagtgatcaagaagaccaacgagaagttccaccagatcgagaaggagtttagcgaggtggagggaaggatccaggacctggagaagtacgtggaggacaccaagatcgatctgtggtcctataatgccgagctgctggtggccctggagaaccagcacaccatcgacctgacagattctgagatgaacaagctgttcgagaggaccaggaagcagctgagggagaacgcagaggacatgggcaatggctgctttaagatctaccacaagtgcgataatgcctgtatcggctccatcagaaacggcacatacgaccacgacgtgtaccgggacgaggccctgaacaatagattccagatcaagggcgtgcagctgaagagcggctataaggattggatcctgtggatctccttcgccatctcttgctttctgctgtgcgtggtgctgctgggctttatctccatgtgggcctgtcagaagggcaacatcagatgcaatatctgtatc

SEQ ID NO: 114 - DNA, 1698 bp, synthetic construct
  source 1..1698: mol_type = other DNA
atgaagaccatcatcgccttcagctgcatcctgtgcctgatctttgcccagaagctgcctggctccgacaattctaccgccaccctgtgcctgggacaccacgcagtgccaaacggcacaatggtgaagaccatcacagacgatcaggtggaggtgaccaatgccacagagctggtgcagagctcctctaccggccggatctgcaatagcccacaccagatcctggatggcaagaactgtacactgatcgactccctgctgggcgatccccactgcgacgatttccagaataaggagtgggacctgtttgtggagaggtctaaggcctacagcaactgttatccctactatgtgcctgattacgccaccctgcgctccctggtggccagctccggcaatctggagttcacccaggagtcttttaactggacaggagtggcacaggacggctctagctacgcatgccggagaggcagcgtgaactccttcttttctagactgaattggctgtataacctgaattacaagtatcctgagcagaacgtgaccatgccaaacaatgacaagttcgataagctgtacatctggggagtgcaccaccctggaaccgacaaggatcagacaaatctgtatgtgcaggccagcggcagagtgatcgtgagcaccaagcggagccagcagacaatcatcccaaacatcggctctcggccctgggtgagaggcgtgtcctctatcatcagcatccactggaccatcgtgaagccaggcgacatcctgctgatcaactccacaggcaatctgatcgcccccagaggctacttcaagatccagaatggcaagagctccatcatgagatccgacgcccacatcgatgagtgcaactctgagtgtatcacccccaacggctctatcagcaatgataagccttttcagaacgtgaataagatcacatacggcgcctgccccaggtatatcaagcagaataccctgaagctggccacaggcatgaggaacatccctgagaagcagacccgcggcatcttcggagcaatcgcaggctttatcgagaacggctgggagggcatggtgaatggctggtatggcttccgccaccagaacagcgagggaaccggacaggcagcagacctgaagtccacccaggccgccatcaaccagatcacaggcaagctgaatagagtgatcaagaagaccaacgagaagttccaccagatcgagaaggagttttctgaggtggagggcagaatccaggatctggagaagtacgtggaggacaccaagatcgatatctggtcctataatgccgagctgctggtggccctggagaaccagcacaccatcgacctgacagatagcgagatgtccaagctgttcgagcggaccaggcgccagctgagagagaacgccgaggacatcggcaatggctgttttaagatctaccacaagtgcgataatagctgtatggagtccatcaggaacggcacatacgaccacgatatctatcggaacgaggccctgaacaatagattccagatcaagggcgtgcagctgaagagcggctataaggactggatcctgtggatctctttcgccatcagctgctttctgctgtgcgtggtgctgctgggctttatcatgtgggcctgccagaggggcaacatccgctgcaatatctgcgtg

SEQ ID NO: 115 - DNA, 1698 bp, synthetic construct
  source 1..1698: mol_type = other DNA
atgaaggccatcctggtggtgctgctgtacacctttaccacagccaacgccgacacactgtgcatcggctatcacgccaacaatagcaccgacacagtggataccgtgctggagaagaatgtgaccgtgacacacagcgtgaacctgctggaggattcccacaatggcaagctgtgcaagctgaggggagtggcaccactgcacctgggcaagtgcaacatcgcaggatggctgctgggaaatccagagtgtgagagcctgtccaccgccagctcctggtcctacatcgtggagacctctaacagcgacaatggcacatgctacccaggcgacttcatcaactatgaggagctgagggagcagctgagcagcgtgagcagcttcgagcggttcgagatcttccccaagaccagctcctggcctaaccacgacaccaataggggagtgacagcagcatgtccacacgcaggaacaaactccttctaccgcaatctgatctggctggtgaagaagggcaattcttaccctaagctgtccaagtcttatatcaacaataaggagaaggaggtgctggtgctgtgggcaatccaccaccccagcacctccgccgaccagcagagcctgtaccagaacgccgatgcctacgtgttcgtgggctctagcaggtactcccgcaagttcgagccagagatcgcaaccaggccaaaggtgagagaccaggccggcaggatgaattactattggacactggtggagcccggcgataagatcacctttgaggccacaggcaacctggtggtgcctcggtatgccttcgccctgaagagaaattctggcagcggcatcatcatctccgatacctctgtgcacgactgcgataccacatgtcagacccctaacggcgccatcaatacaagcctgcctttccagaacatccacccagtgacaatcggcgagtgccctaagtacgtgaagtccaccaagctgaggatggccacaggcctgcgcaatatcccaagcatccagtcccgcggcctgtttggagcaatcgcaggcttcatcgagggaggatggaccggaatgatcgacggatggtacggctatcaccaccagaacgagcagggctctggctatgccgccgacctgaagagcacccagaatgccatcgatggcatcacaaacaaggtcaatagcgtgatcgagaagatgaacacccagtttacagccgtgggcaaggagttcaataagctggagcggagaatggagaacctgaataagaaggtggacgatggctttctggatatctggacctacaacgccgagctgctggtgctgctggagaatgagcggacactggactaccacgattccaacgtgaagaatctgtatgagaaggtgagatctcagctgaagaacaatgccaaggagatcggcaacggctgcttcgagttttaccacaagtgcaacaatgagtgtatggagtctgtgaagaacggcacctacgactatcccaagtatagcgaggaggccaagctgaatcgggaggagatcgatggcgtgaagctggagagcaccagaatctaccagatcctggccatctattccacagtggcctcctctctggtgctgctggtgtccctgggcgccatctctttctggatgtgctctaacggcagcctgcagtgccggatctgtatc

SEQ ID NO: 116 - DNA, 1695 bp, synthetic construct
  source 1..1695: mol_type = other DNA
atgaaggtgaagctgctgatcctgctgtgcacctttacagccacctacgccgacacaatctgtatcggctatcacgccaacaattccacagacaccgtggataccatcctggagaagaacgtgacagtgacccactctgtgaatctgctggagaacaggcacaatggcaagctgtgcctgctgaagggaatcgcaccactgcagctgggaaactgcagcgtggcaggatggatcctgggcaatcctgagtgtgagtctctgatcagcaagaagtcctggtcttacatcgtggagacacccaaccctgagaatggcgcctgctaccccggcgagtttgccgattatgaggagctgagggagcagctgagcaccgtgagcagcttcgagcggttcgagatcttcccaaaggagtctagctggcccaaccacacagtgaccggcgtgagcgcctcctgttctcacaacggcgagaggtccttctaccgcaatctgatctggctgaccgtgaagaacggcctgtaccctaatctgagcaagtcctatgagaatgacaagggcaaggaggtgctgatcctgtggggcgtgcaccacccatccaacatcggcgatcagcgcacactgtaccacaccgagaatgcctacgtgagcgtggtgtcctctcactacagccggagattcacacctgagatcaccaagcggccaaaggtgagagaccaggagggccggatcaactactattggacactgctggagcctggcgataccatcatctttgaggccaacggcaatctgatcgccccatggtatgccttcgccctgagcagaggcctgggatccggcatcatcacatctaatgccccaatggacgagtgcgatgccaagtgtcagaccccacagggagccatcaacagctccctgccctttcagaatgtgcaccctgtgacaatcggcgagtgtccaaagtacgtgaggagcgccaagctgaggatggtgaccggactgaggaacgtgccctccatccagtctagaggcctgctgggagcaatcgcaggcttcatcgagggaggatggacaggaatggtggacggatggtacggctatcaccaccagaatggacagggatccggatatgcagcagatcaggagtctacacagaacgccatcaatggcatcaccaacaaggtcaatagcatcatcgagaagatgaacatccagtttacctccgtgggcaaggagttcaatcacctggagaagaggatcgagaacctgaataagaaggtggacgatggctttctggacgtgtggacatacaacgccgagctgctgatcctgctggagaatgagcgcaccctggacttccacgattccaacgtgaagtctctgtatgagaaggtgaagagccagctgaagaacaatgccaaggaagtgggcaatggctgcttcgagttttaccacaagtgcgacgatacatgtatggagtccgtgaagaacggcacctacaattatcctaagtatagcgaggagtccaagctgaaccgggagaagatcgacggcgtgaagctggattctatgggcgtgtacagaatcctggccatctatagcaccgtggcctctagcctggtgctggtggtgagcctgggagcaatctccttttggatgtgctctaacggcagcctgcagtgccgggtgtgcatc

SEQ ID NO: 117 - DNA, 1698 bp, synthetic construct
  source 1..1698: mol_type = other DNA
atggaggccaagctgttcgtgctgttttgcgccttcaccgccctgaaggccgacacaatctgcgtgggctaccacgccaacaatagcaccgagacagtggataccgtgctggagaagaacgtggccgtgacacactccgtgaacctgctggaggacaagcacaatggcaagctgtgcaagctgggaggaatcgcaccactgcacctgggcaagtgcaacatcgccggctggatcctgggcaatcctgagtgtgagctgctgatcagcaaggagtcttggagctacatcgtggaggccccaaactccgacaatggagcatgctacccaggacagttcgcagattatgaggagctgcgggagcacctgagctccgtgtctagctttgagaagttcgagatctttccaaaggcctcctcttggcccaaccacgattctaataagggagtgaccgcagcatgtccttacgcaggcgccaacagcttttataggaatctgctgtggatcgtgaagaagggcaacagctacccaaagatcaataagtcctacatcaacaataagggcaaggaggtgctggtgctgtggggaatccaccacccacctacctccacagaccagcagtctctgtacaagaacgccgatgcctacgtgttcgtgggcacctcccggtactctaagaagtttaagcctgagatcgccatcaggccaaaggtgcgcgaccaggagggcagaatgaattactattggaccctgctggaccagggcgataccatcacattcgaggcaacaggaaacctggtggcaccaagatatgcctttgccatggagcgcaatgccggcagcggcatcatcatctccgacacccctgtgcacgattgcaacaccacatgtcagacaccaaagggcgccatcaacagcaatctgcccttccagaatgtgcaccctatcaccatcggcgagtgtccaaagtacgtgcggtccaccaagctgagaatggtgacaggcctgcggaacatccccagcgtgcagtccagaggcctgttcggagcaatcgcaggctttctggagggaggatggacaggcatggtgaacggctggtacggctatcaccaccagaatgaccagggctctggctatgccgccgatcagaagagcacccagatcgccatcgacggcatctctaacaaggtcaatagcgtgatcgagaagatgaatatccagttcacagccgtgggcaaggagtttagccacctggagcggagaatcgagaacctgaataagaaggtggacgatggcctgctggacgtgtggacctacaacgccgagatgctggtgctgctggagaatgagaggacactggacttccacgacttcaacgtgaagaatctgtacgagaaggtgaagtcccagctgcgcaacaatgccaaggagatcggcaacggctgcttcgagttttatcacaagtgcgacaacacctgtatggagtccgtgaggaatggcacatacgattatcctaagtactatgaggagtctaagctgaatcgcgaggagatcgatggcgtgaagctggagtccatgggcgtgtaccagatcctggccatctattctaccgcagccagctccctggtgctgctggtgtccctgggagccgtgtctttctggatgtgctctaacggcagcctgcagtgcaagatctgtatc

SEQ ID NO: 118 - DNA, 1713 bp, synthetic construct
  source 1..1713: mol_type = other DNA
atgaagaccatcatcgccttctcctgcatcctgtgcctgatcttcgcccagaagctgcccggcagcgacaacaactccatggccaccctgtgcctgggacaccacgccgtgccaaacggcaccctggtgaagaccatcaccgacgaccagatcgaggtgaccaacgccaccgagctggtgcagtccagctccaccggaaggatctgcaacagcccaggacaccagatcctggacggcaagaactgcaccctgatcgacgctctgctgggcgacccacactgcgacgacttccagaacaagtgggacctgttcgtggagcgcagcaccgcctactccggcaactgctacccctactacgtgccagactacgccagcctgagggagcagctgtccctggtggctagctccggcaccctggagttcacccaggagtccttcaactggaccggcgtggcccaggacggaagctcctacgcttgcaggaggggaagcgtgaacaacagcttcttctccagactgaactggctgtacctgaactacaagtaccccgccctgaacgtgaccatgccaaacaacgacaagttcgacaagctgtacatctggggagtgcaccacccaggcaccgacaaggaccagaccaacctgtacgtgcaggcctccggaagggtggtgagcaccaagagatcccagcagaccgtgatccccaacatcggatccaggccatgggtgcggggcgtgagctccatcatcagcatctactggaccatcgtgaagcccggcgacatcctgctgatcaactccaccggcaacctgatcgccccaaggggctacttcaagatccagagcggcaagagctccggcatcatgagaagcgacgcccccatcgactgcaactccgagtgcatcaccccaaacggcagcatccccaacgacaagccattccagaacgtgaacaggatcacctacggcgcctgccccagatacgtgaagcagaacaccctgaagctggccaccggcatgcgcaacgtgccagagaagcagaccaggggaatcttcggagctatcgctggcttcatcgagaacggctgggagggcatggtggacggctggtacggcttccgccaccagaactccgagggcaccggacaggctgctgacctgaagagcacccaggccgccatcaaccagatcaccggcaagctgaaccgcgtgatcaagaagaccaacgagaagttccaccagatcgagaaggagttctccgaggtggagggcagaatccaggacctggagaagtacgtggaggacaccaagatcgacctgtggtcctacaacgccgagctgctggtggccctggagaaccagcacaccatcgacctgaccgacagcgagatgaacaagctgttcgagaggaccagaaagcagctgcgcgagaacgccgaggacatgggcaacggctgcttcaagatctaccacaagtgcgacaacgcctgcatcggcagcatccggaacggcacctacgaccacgacgtgtaccgcgacgaggccctgaacaaccggttccagatcaagggcgtgcagctgaagtccggctacaaggactggatcctgtggatcagcttcgccatctcctgcttcctgctgtgcgtggtgctgctgggcttcatcagcatgtgggcctgccagaagggcaacatccggtgcaacatctgcatc

SEQ ID NO: 119 - DNA, 1698 bp, synthetic construct
  source 1..1698: mol_type = other DNA
atgaagaccatcatcgccttctcctgcatcctgtgcctgatcttcgcccagaagctgccaggaagcgacaactccaccgccaccctgtgcctgggacaccacgccgtgccaaacggcaccatggtgaagaccatcaccgacgaccaggtggaggtgaccaacgccaccgagctggtgcagtccagctccaccggaaggatctgcaactccccacaccagatcctggacggcaagaactgcaccctgatcgacagcctgctgggcgacccacactgcgacgacttccagaacaaggagtgggacctgttcgtggagaggtccaaggcctacagcaactgctacccctactacgtgccagactacgccaccctgagatccctggtggccagctccggcaacctggagttcacccaggagagcttcaactggaccggcgtggcccaggacggaagctcctacgcttgcaggaggggcagcgtgaactccttcttcagccggctgaactggctgtacaacctgaactacaagtaccccgagcagaacgtgaccatgccaaacaacgacaagttcgacaagctgtacatctggggagtgcaccacccaggcaccgacaaggaccagaccaacctgtacgtgcaggcctccggacgcgtgatcgtgtccaccaagagaagccagcagaccatcatccccaacatcggatccaggccatgggtgcggggcgtgagctccatcatcagcatccactggaccatcgtgaagcccggcgacatcctgctgatcaacagcaccggaaacctgatcgctccaaggggctacttcaagatccagaacggcaagagctccatcatgcggtccgacgcccacatcgacgagtgcaacagcgagtgcatcacccccaacggcagcatctccaacgacaagccattccagaacgtgaacaagatcacctacggcgcctgccccaggtacatcaagcagaacaccctgaagctggccaccggcatgaggaacatcccagagaagcagaccagaggcatcttcggcgccatcgccggcttcatcgagaacggctgggagggcatggtgaacggctggtacggcttcagacaccagaactccgagggcaccggacaggctgctgacctgaagagcacccaggccgccatcaaccagatcaccggcaagctgaaccgcgtgatcaagaagaccaacgagaagttccaccagatcgagaaggagttctccgaggtggagggaaggatccaggacctggagaagtacgtggaggacaccaagatcgacatctggagctacaacgccgagctgctggtggccctggagaaccagcacaccatcgacctgaccgactccgagatgagcaagctgttcgagcgcaccaggagacagctgcgggagaacgccgaggacatcggcaacggctgcttcaagatctaccacaagtgcgacaactcctgcatggagagcatcaggaacggcacctacgaccacgacatctaccgcaacgaggccctgaacaaccggttccagatcaagggcgtgcagctgaagagcggctacaaggactggatcctgtggatctccttcgccatcagctgcttcctgctgtgcgtggtgctgctgggcttcatcatgtgggcctgccagaggggcaacatcagatgcaacatctgcgtg

SEQ ID NO: 120 - DNA, 1698 bp, synthetic construct
  source 1..1698: mol_type = other DNA
atgaagaccatcatcgccttctcctgcatcctgtgcctgatcttcgcccagaagctgcccggcagcgacaactccatggccaccctgtgcctgggacaccacgccgtgccaaacggcaccctggtgaagaccatcaccgacgaccagatcgaggtgaccaacgccaccgagctggtgcagtccagctccaccggcaggatctgcaacagcccccaccagatcctggacggcaagaactgcaccctgatcgacgctctgctgggcgacccacactgcgacgacttccagaacaaggagtgggacctgttcgtggagcgcagcaccgcctacagcaactgctacccctactacgtgccagactacgccagcctgcggtccctggtggctagctccggcaccctggagttcacccaggagtccttcaactggaccggcgtggcccaggacggaagctcctacgcttgcaggaggggcagcgtgaactccttcttcagcagactgaactggctgtacaacctgaactacaagtaccccgagcagaacgtgaccatgccaaacaacgacaagttcgacaagctgtacatctggggagtgcaccacccaggcaccgacaaggaccagaccaacctgtacgtgcaggcctccggacgcgtgatcgtgtccaccaagagaagccagcagaccgtgatccccaacatcggatccaggccatgggtgcggggcgtgagctccatcatcagcatctactggaccatcgtgaagcccggcgacatcctgctgatcaacagcaccggcaacctgatcgccccaaggggctacttcaagatccagtccggcaagagctccatcatgagaagcgacgcccacatcgacgagtgcaactccgagtgcatcacccccaacggcagcatccccaacgacaagccattccagaacgtgaacaggatcacctacggcgcctgccccagatacgtgaagcagaacaccctgaagctggccaccggcatgcgcaacgtgccagagaagcagaccaggggaatcttcggagctatcgctggcttcatcgagaacggctgggagggcatggtggacggctggtacggcttccgccaccagaactccgagggcaccggacaggctgctgacctgaagagcacccaggccgccatcaaccagatcaccggcaagctgaaccgcgtgatcaagaagaccaacgagaagttccaccagatcgagaaggagttcagcgaggtggagggcagaatccaggacctggagaagtacgtggaggacaccaagatcgacctgtggtcctacaacgccgagctgctggtggccctggagaaccagcacaccatcgacctgaccgacagcgagatgaacaagctgttcgagaggaccagaaagcagctgcgcgagaacgccgaggacatgggcaacggctgcttcaagatctaccacaagtgcgacaacgcctgcatcggctccatccggaacggcacctacgaccacgacgtgtaccgcgacgaggccctgaacaaccggttccagatcaagggcgtgcagctgaagagcggctacaaggactggatcctgtggatctccttcgccatcagctgcttcctgctgtgcgtggtgctgctgggcttcatcatgtgggcctgccagaagggcaacatccggtgcaacatctgcatc

SEQ ID NO: 121 - DNA, 1698 bp, synthetic construct
  source 1..1698: mol_type = other DNA
atgaagaccatcatcgccctgtcctacatcctgtgcctggtgttcgcccagaagctgccaggaaacgacaacaacatggccaccctgtgcctgggacaccacgccgtgcccaacggcaccatcgtgaagaccatcaccaacgaccagatcgaggtgaccaacgccaccgagctggtgcagtccaccagcaagggcgagatctgctccagcccacaccagatcctggacggcgagaactgcaccctgatcgacgctctgctgggcgacccacactgcgacgacttccagaacaagaagtgggacctgttcgtggagagatccaaggcccacagcaactgctacccctactacgtgccagactacgccaccctgcgctccctggtggcttccagcggcaacctggagttcacccaggagagcttcaactggaccggagtgacccaggacggagcttccagcgcctgcaagcgccggtccagcaagtccttcttcagccgcctgaactggctgcacaacctgaactacaagtacccagccctgaacgtgaccatgcccaacaacgacaacttcgacaagctgtacatctggggcgtgcaccacccatccaccgacaaggaccagacctccctgtacatccaggccagcggaagggtgaccgtgtccaccaagaggagccagcaggccgtgatcccaaacatcggatccaggccacgggtgagggacatcccctcccggatcagcatctactggaccatcgtgaggccaggcgacatcctgctgatcaacagcaacggcaacctgatcgcccccagaggctacttcaagatccgctccggcaagtccagcatcatgcggagcgacgcccccatcggcaactgcaacagcgagtgcatcaccccaaacggctccatcagcaacgacaagcccttccagaacgtgaacaagatcacctacggcgcctgcccaaggtacatcaagcagaacaccctgaagctggccaccggcatgagaaacatccccgagaagcagaccaggggaatcttcggagctaaggctggcttcatcgagaacggctgggagggcatggtgaacggctggtacggcttcagacaccagaactccgagggaaggggacaggctgctgacctgaagagcacccaggccgccatcgaccagatcaacggcaagctgaacagactgatcggcaagaccaacgagaagttccaccagatcgagaaggagttctccgagatcgagggccggatccaggacctggagaggtacgtggaggacaccaagatcgacatctggagctacaacgccgagctgctggtggccatggagaaccagcacaccatcgacctgaccgactccgagatgagcaagctgttcgagagaaccaggagacagctgcgcgagaacgccgaggacatgggcaacggctgcctgaagatctaccacaagtgcgacaactcctgcatggagagcatccggaacggcacctacgaccacgacatctaccggaacgaggccctgaacaacaggttccagatcaagggcgtggagctgaagagcgagtacaaggactggatcctgtggatctccttcgccaccagctgcttcctgctgtgcgccgtgctgctgggcttcatcatgtgggcctgccagaagggcaacatcaagtgcaacatctgcatc

SEQ ID NO: 122 - DNA, 1698 bp, synthetic construct
  source 1..1698: mol_type = other DNA
atgaaggccatcatcgccttctcctgcatcctgtgccagatcttcgcccagaagctgcccggcaacgacaactccaccgccaccctgtgcctgggacaccacgccgtgagcaacggcaccctggtgaagaccatcaccgacgaccaggtggaggtgaccaacgccaccgagctggtgcagaacttctccatgggcaagatctgcaagaacccccacaggatcctggacggcgccaactgcaccctgatcgacagcctgctgggcgacccacactgcgacggcttccagaacgagaagtgggacctgttcatcgagaggtccaaggccttcagcaactgctacccctacgacgtgccagactacgcctccctgagaagcctgatcgcctccagcggcaccctggagttcaacaacgagtccttcaactggaccggagtgacccagaacggaggatccagcgcctgcaagaggggacccaacaactccttcttcagcagactgaactggctgacccacctgaacttcaagtaccccgccctggaggtgaccatgccaaacaacgagcagttcgacaagctgtacatctggggagtgcaccacccagctaccgacaaggaccagatcagcctgtacgctcaggctgctggaaggatcatcgtgtccaccaagagaagccagcagaccatcatccccaacatcggctccaggccatggatcagaggcgtgtccagcatcatcagcatccactggaccatcgtgaagcccggcgacatgctgctgatcaactccaccggcaacatcatcgccccacgcggctacttcaagatccagaccggcaagtccagcgtgatgcggagcgacgctccaatcggcaagtgcaactccgcctgcatcaccccaaacggaagcatccccaacgaccgcccattccagaacgtgaacaagatcacctacggcgcctgccccaagtacgtgaagcagaacaccctgaagctggccaccggcatgaggaacatcccagagaggcagaccaggggaatcttcggagctatcgccggcttcatcgagaacggctgggagggcatgatcgacggctggtacggcttccgccaccacaactccgagggcaccggacaggctgctgacatcaagagcacccaggctgccatcaaccagatcaacggcaagctgaaccgcgtgatcgagaagaccaacgagaagttccaccagatcgagaaggagttcctggaggtggagggccggatccaggacatcgagaagtacatcgaggacaccaagatcgacctgtggtcctacaacgccgagctgctgatcgccctggagaaccagcacaccatcgacctggccgacagcgagatgaacaagctgttcgagaagaccaagaagcagctgagggagaacgccgaggacatcggcaacggctgcttcaagatctaccacaagtgcgacaacacctgcatcggcagcatcagaaacggcacctacaaccacgacgtgtaccgcgacgaggccgtgaacaaccggttccagatcaagggcgtgcagctgaagctgggctacaaggactggatcctgtggatctccttcgccatgagctgcttcctgctgtgcgtggccctgctgggcttcatcatgtgggcttgccagaggggaaacatcaggtgcaacatctgcgtg

(19) United States
(12) Patent Application Publication
(10) Pub. No.: US 2025/0032601 A1
(43) Pub. Date: Jan. 30, 2025

(54) METHODS OF MAKING AND USING UNIVERSAL CENTRALIZED INFLUENZA VACCINE GENES

(71) Applicant: NUtech Ventures, Lincoln, NE (US)

(72) Inventor: Eric Anthony Weaver, Lincoln, NE (US)

(21) Appl. No.: 18/800,951

(22) Filed: Aug. 12, 2024

Related U.S. Application Data
(63) Continuation of application No. 17/277,426, filed on Mar. 18, 2021, now Pat. No. 12,064,478.
(60) Provisional application No. 62/734,791, filed on Sep. 21, 2018.

(51) Int. Cl. A61K 39/145; A61K 39/00; C12N 7/00

(52) U.S. Cl. CPC A61K 39/145; C12N 7/00; A61K 2039/55555; C12N 2710/10034; C12N 2740/10034; C12N 2750/14134; C12N 2760/18034; C12N 2760/20034; C12N 2770/36134

(57) ABSTRACT

This document describes a number of different polypeptide sequences, and the nucleic acid sequences encoding such polypeptide sequences, that can be used alone or in combination as universal vaccines against viruses including influenza A or influenza B in humans or influenza in swine.

### CROSS REFERENCE TO RELATED APPLICATIONS

This application is a Continuation application of U.S. application Ser. No. 17/277,426 filed Mar. 18, 2021, which is a National Stage Application under 35 U.S.C. § 371 and claims the benefit of priority to International Application No. PCT/US2019/052137, filed Sep. 20, 2019, which claims the benefit of U.S. Provisional Application No. 62/734,791, filed on Sep. 21, 2018. The contents of such applications are incorporated by reference in their entireties.

### INCORPORATION BY REFERENCE

This application contains a Sequence Listing that has been submitted electronically as an XML file named “24742-0115002_SL_ST26.xml.” The XML file, created on Sep. 19, 2024, is 193,773 bytes in size. The material in the XML file is hereby incorporated by reference in its entirety.

### TECHNICAL FIELD

This disclosure generally relates to vaccines and, specifically, influenza vaccines.

### BACKGROUND

Influenza virus was identified as the causative agent for febrile illness in swine in 1931. Influenza virus in humans was discovered two years later in 1933.

Annually, 5-15% of the world's population is affected by epidemics and have upper respiratory tract infections, 3 to 5 million have severe illness, and 250,000 to 500,000 cases result in death (WHO 2009). In the U.S., seasonal influenza affects up to 20% of the population, results in 200,000 hospitalizations and approximately 37,000 deaths each year. The World Health Organization (WHO) states, “influenza rapidly spreads around the world in seasonal epidemics and imposes a considerable economic burden in the form of hospital and other health care costs and lost productivity. In the United States of America, it is estimated that influenza epidemics cost up to $167 billion per year.”

While these seasonal infections are certainly of concern, pandemic influenza outbreaks and intentional releases of pathogenic influenza are of substantially higher concern. During the past century, there have been several severe pandemics (WHO 2009). In 1918-1919, an H1N1 known as the Spanish Flu caused the world's largest influenza pandemic, killing 20-40 million people. In 1957, the Asian flu caused by an H2N2 virus resulted in ˜1.5 million deaths, and in 1968, the Hong Kong flu caused by an H3N2 influenza resulted in ˜1 million deaths.

Prior to the mid-1990s, the primary circulating virus strains in pigs was classical swine influenza A H1N1. Since 1997, several new strains of swine influenza virus have emerged, including H1N1, H3N2 and HIN2. The emerging triple reassortants were combinations of avian, human and swine influenza genes. In March of 2009, swine influenza emerged in Mexico and in both California and Texas in the United States. This novel swine flu circulated the globe and infected 24% of the world's human population.

Since swine are susceptible to human, avian and swine influenza, they act as a mixing vessel to create new reassorted influenza viruses. Not only is there a risk of zoonosis from influenza infected swine, Influenza A virus of swine (IAV-S) is one of the most important pathogens of swine. The virus is widespread worldwide, causing tremendous economic loss to swine producers. Clinically, pigs infected with IAV-S often display signs of an acute respiratory disease, which is rapidly resolved after 7-10 days. However, when associated with other pathogens of the porcine respiratory disease complex, IAV-S infection in pigs often leads to severe pneumonia and even to death. One study estimated the losses due to swine influenza resulted in approximately $700,000,000 in losses for the United States swine industry.

Thus, there is a significant need for an influenza vaccine that is effective against divergent influenza virus strains. This disclosure provides for methods of making and using universal centralized influenza vaccine genes.

### SUMMARY

The universal vaccine genes described herein were created using several unique strategies that are described herein and were computationally optimized to provide the greatest possible vaccine efficacy. These unique sequences can allow for the production of a universal vaccine for the prevention of influenza virus infections that provides near-complete protection against all divergent influenza virus strains.

In one aspect, a vaccine polypeptide having at least 90% sequence identity (e.g., at least 95% sequence identity; at least 99% sequence identity; or 100% sequence identity) to an amino acid sequence selected from the group consisting of any of SEQ ID NOs: 1-102 is provided.

In some embodiments, the vaccine polypeptide has at least 90% sequence identity (e.g., at least 95% sequence identity; at least 99% sequence identity; or 100% sequence identity) to an amino acid sequence selected from the group consisting of SEQ ID NOs: 6-8, 13-15, 20-22, 27-29, 34-36, 41-43, 49-52, 57-60, 65-67, 72-74, 79-81, 86-88, 93-95, and 100-102.

In some embodiments, the vaccine polypeptide has at least 90% sequence identity (e.g., at least 95% sequence identity; at least 99% sequence identity; or 100% sequence identity) to an amino acid sequence selected from the group consisting of SEQ ID NOs: 3, 6, 17, 20, 46, 49, 54, and 57.

In some embodiments, the polypeptide is encoded by a nucleic acid sequence having at least 90% sequence identity (e.g., at least 95% sequence identity; at least 99% sequence identity; or 100% sequence identity) to a nucleic acid sequence selected from the group consisting of SEQ ID NO: 103-122.

In another aspect, a vaccine composition comprising at least one of the vaccine polypeptides described herein is provided along with a delivery vehicle. In some embodiments, the delivery vehicle is a virus. Representative viruses include, without limitation, an adenovirus, an adeno-associated virus, a retrovirus, an alphavirus, a paramyxovirus, or a rhabdovirus. In some embodiments, the delivery vehicle is a pharmaceutically acceptable carrier. A representative pharmaceutically acceptable carrier is saline. In some embodiments, the delivery vehicle is a nanoparticle (e.g., a lipid nanoparticle). In some embodiments, a vaccine composition includes at least two vaccine polypeptides, at least three vaccine polypeptides, at least four vaccine polypeptides, at least eight vaccine polypeptides, or at least twelve vaccine polypeptides.

In yet another aspect, a method of vaccinating a subject is provided. Such a method typically includes administering a centralized vaccine polypeptide as described herein or a vaccine composition as described herein to a subject in need of vaccination. Representative subjects include a human and a swine. In some embodiments, the administering step is repeated more than once.

In some embodiments, a vaccine polypeptide as described herein is a universal vaccine against at least about 3 different viral sub-types. In some embodiments, a vaccine polypeptide as described herein is a universal vaccine against at least about 12 viral sub-types.

### Definitions

Antigen and immunogen are used interchangeably throughout this disclosure. Both refer to antigenic proteins that induce immune responses.

A universal vaccine refers to a vaccine that can provide protection against most variants within an influenza subtype and subtypes within both group 1 and 2 influenza viruses. A universal vaccine can provide protection using multiple immunogens.

A centralized sequence refers to a sequence that mimics an ancestor of influenza infections during past years. Centralized sequences (e.g., polypeptides) are designed to localize to the center of a phylogenetic tree of a subtype of influenza virus. The centralized sequences described herein were created using individual wild type sequences that represent the major branches of the representative phylogenetic tree. The rationale for this approach was to produce an immunogen that is centrally located with respect to all other variants. Such a protein then has lower sequence divergence with all of the variants than any two randomly selected genes.

Unless otherwise defined, all technical and scientific terms used herein have the same meaning as commonly understood by one of ordinary skill in the art to which the methods and compositions of matter belong. Although methods and materials similar or equivalent to those described herein can be used in the practice or testing of the methods and compositions of matter, suitable methods and materials are described below. In addition, the materials, methods, and examples are illustrative only and not intended to be limiting. All publications, patent applications, patents, and other references mentioned herein are incorporated by reference in their entirety.

### DESCRIPTION OF DRAWINGS

FIG. 1 is a schematic showing the phylogenetic analyses of centralized hemagglutinin genes. The genetic relationships between the consensus genes and wild type genes are shown in unrooted neighbor-joining phylogenetic trees. H1, H2, H3 and H5 consensus COT genes are shown localizing to the center of the tree. Black bars indicate the percent divergence of the HA amino acid sequences. The central tree (dotted line) illustrates the degree of genetic diversity between the HA subtypes.

FIG. 2 is a graph showing protection against influenza virus infection by wild type and centralized HA immunogens. Mice were immunized intramuscularly with A/PR/8/34, H1-COT, and A/CA/04/09 HA expressing adenovirus. Three weeks after immunization, the mice were challenged intranasally with 100 LD50 of influenza virus A/PR/8/34 (left), A/FM/1/47 (center) or 70 LD50 of A/CA/04/09 (right). Individual mouse weights for the vaccinated mice and the mean and standard error of the control DPBS immunized mice are shown. Mice exhibiting profound signs of disease and less than 75% of baseline weights were humanely sacrificed.

FIG. 3 is a schematic of mosaic and epigraph vaccine immunogen construction. This diagram shows how novel mosaic Influenza proteins are constructed. All of the intra-subtype hemagglutinin (HA) or neuraminidase (NA) proteins are aligned and used as natural sequences to create Mosaic sequences. Two sequences are randomly chosen and a two-point recombination is created (Step 1). The new recombinant is evaluated and if “fitness” has increased it is placed back into the protein pool (Step 2). This process is repeated for a defined number of generations, usually millions, in order to create a set of mosaic proteins that mimic naturally derived novel recombinants (Step 3). As the process continues the proteins that have the best coverage are chosen ultimately resulting in the Best-Fit Mosaic Protein (Step 4). Ultimately, the Mosaic vaccine immunogen is centralized to the phylogenetic tree and represents all of the wild type proteins equally, with a bias toward potential T and B cell epitopes (PTBEs).

FIG. 4 shows the vaccine coverage of five different vaccine immunogen approaches. The blue circles represent predicted coverage for vaccine protection and the red circles represent the predicted coverage for complete sterilizing immunity induced by each of the vaccine immunogens. Vaccines based on wild type immunogens are very strain-specific and produce little, if any, cross-protective immunity (A). A centralized consensus vaccine is designed to localize to the center of the phylogenetic tree and represent all wild type strains equally (B). We have found that, indeed, these vaccine immunogens provide greater cross-protective immunity as compared to wild type genes when the vaccines are mismatched. A second consensus approach is to create a consensus of unique sequences (COUS), which gives equal weight to all unique influenza variants and gives bias towards more contemporary strains (C). Another immunogen approach is shown as a mosaic immunogen. The mosaic vaccine approach uses computational recombination over millions of generations to select for the single best vaccine immunogen with the greatest ≥9-mer coverage as compared to the average of all wild type sequences. Single mosaic immunogens also localize near the center of the tree as they are designed to be represent all known wild type sequences (D). The epigraph immunogens are designed in the same way as the mosaic immunogens with the exception that this is a multi-immunogen approach. The epigraph-designed immunogens focus on clusters of protein sequences to create a cocktail of immunogens that produce the greatest level of coverage across the designated number of immunogens requested in the cocktail. The design described herein focused on the use of a three-immunogen cocktail to give the greatest level of coverage without the burden of too many sequences (E).

FIG. 5 shows a phylogenetic analysis of H1 HA proteins created by Consensus centralized Center of Tree (COT), Consensus of Unique Sequences (CoUS), Mosaic (Mos) and Epigraph (Epi) computational approaches. 7313 unique human H1 Influenza virus proteins were downloaded and laboratory strains and duplicate sequences were excluded. In order to show where the sequences localize on the phylogenetic tree, only representative sequences were chosen that show sequence evolution from years 1918 to 2018. The Mosaic Vaccine Design and Epigraph programs that are available through the HIV database tools suite were used to create the Mosaic and Epigraph candidate vaccine proteins, respectively. The consensus COT vaccine protein was designed to localize to the center of the tree. The Consensus CoUS candidate vaccine protein was computationally derived using the Consensus Maker Software. Both the CoUS and Mosaic proteins localize to the center of the main Influenza H1 cluster, which represents the majority of H1 diversity. These two candidate vaccine immunogens represent the average or ancestral protein from which all future influenza strains would evolve. The two major exceptions to the approaches are that the Consensus protein simply represents the most conserved amino acid sequence at each position, while the mosaic also seeks to represent the most commonly repeated sequences and it is also biased towards >9-mer motifs that maximally optimize the potential T and B cell epitopes (PTBE). The epigraph software is also designed to maximize the PTBEs within the final vaccine candidates, however, it is also designed to identify multiple Epigraph protein sequences that maximally cover the widest range of diversity within the influenza cluster. Indeed, the Epigraph 2 protein also localizes to the main cluster of all human Influenza H1N1 viruses analyzed, whereas the Epigraph 1 protein localizes to the central region of the pd09. In order to maximize coverage of sequences in between clusters, a third Epigraph 3 protein was created. This third protein is a unique recombinant of proteins in which short segments are synthetically recombined to make a montage protein sequence that represents viruses of classical and pd09 ancestry. The Consensus, Mosaic, and Epigraph proteins are shown in green, blue and yellow, respectively. The FluZone protein is shown in red. The challenge viruses and viruses for immune correlates are shown in purple and black, respectively.

FIG. 6 are graphs showing sum T cell responses to several HA vaccine candidates. In order to determine if the proposed strategies would induce greater levels of T cell responses, the ability of the consensus CoUS and Mosaic vaccine strategies compared to two wild type HA vaccines. All four HA vaccine immunogens were cloned into an Adenovirus expression system and groups of five mice were immunized with 108 vp/mouse. Three weeks after immunization, the splenocytes were harvested and the total T cells responses to four divergent influenza viruses was determined using an overlapping peptide library. Interferon-gamma secreting cells were detected using an ELISpot assay. The green bars indicate that the vaccine and the peptide pools are matched.

FIG. 7 is a schematic showing the epitope mapping of vaccines. The splenocytes from Mosaic, CoUS, PR8 and pd09 HA vaccinated mice were screened for induced T cell epitopes using a Brisbane/59/07 peptide library and the positive peptides were represented by red, green, purple and yellow bars, respectively.

FIG. 8 is a Western blot analysis of vaccine vectors. The adenovirus vaccine vectors were analyzed for protein expression. 293 cells were infected with equivalent infectious units and protein was detected by probing the blots with polyclonal Goat anti-A/swine/Indiana/0392/2011 serum.

FIG. 9 shows the phylogenetic relationship between the vaccine genes described herein and various strains of human influenza A H1.

FIG. 10 shows the phylogenetic relationship between the vaccine genes described herein and various strains of human influenza A H2.

FIG. 11 shows the phylogenetic relationship between the vaccine genes described herein and various strains of human influenza A H3.

FIG. 12 shows the phylogenetic relationship between the vaccine genes described herein and various strains of human influenza A H3.

FIG. 13 shows the phylogenetic relationship between the vaccine genes described herein and various strains of human influenza A H5.

FIG. 14 shows the phylogenetic relationship between the vaccine genes described herein and various strains of human influenza A N1.

FIG. 15 shows the phylogenetic relationship between the vaccine genes described herein and various strains of human influenza A N2.

FIG. 16 shows the phylogenetic relationship between the vaccine genes described herein and various strains of human influenza B HA Victoria.

FIG. 17 shows the phylogenetic relationship between the vaccine genes described herein and various strains of human influenza B HA Yamagata.

FIG. 18 shows the phylogenetic relationship between the vaccine genes described herein and various strains of human influenza B NA Victoria.

FIG. 19 shows the phylogenetic relationship between the vaccine genes described herein and various strains of human influenza B NA Yamagata.

FIG. 20 shows the phylogenetic relationship between the vaccine genes described herein and various strains of swine influenza A H1.

FIG. 21 shows the phylogenetic relationship between the vaccine genes described herein and various strains of swine influenza A H3.

FIG. 22 shows the phylogenetic relationship between the vaccine genes described herein and various strains of swine influenza A N1.

FIG. 23 shows the phylogenetic relationship between the vaccine genes described herein and various strains of swine influenza A N2.

FIG. 24A shows a neighbor-joining phylogenetic tree of all unique H1 HA protein sequences with representative strains labelled. Pandemic 2009 (pdm09) strains are shown in dark blue.

FIG. 24B is a schematic of HA amino acid sequences submitted to SWISS-MODEL server and visualized with PyMOL software to predict the protein folding of each immunogen. The predicted structures of the mosaic (blue) and wild type comparator HA (grey and purple) for both the head (dark) and stalk (light) regions are shown along with a merged image superimposing all three HA structures.

FIG. 24C is a Western blot showing expression of the Mosaic and the two wild type comparator HA sequences (A/PR/8/34 and A/TX/05/09) in recombinant Adenovirus type 5.

FIG. 25A-25E are graphs showing hemmaglutination inhibition (HI) titers after single shot vaccination of mice with Influenza strains A/CA/07/09 (FIG. 25A), A/NC/20/99 (FIG. 25B), A/PR/8/34 (FIG. 25C), A/WS/33 (FIG. 25D), and A/FM/1/47 (FIG. 25E). Data is expressed as the mean with standard error (SEM).

FIG. 25F is a heat map of the HI titer responses, which reports the corresponding percent similarity between the vaccine group and virus (****p<0.0001, ***p<0.001; one-way ANOVA with Bonferroni multiple comparisons).

FIG. 26A-26E are graphs showing HI titers after vaccination of mice and boost with Influenza strains A/CA/07/09 (FIG. 26A), A/NC/20/99 (FIG. 26B), A/PR/8/34 (FIG. 26C), A/WS/33 (FIG. 26D), and A/FM/1/47 (FIG. 26E).

FIG. 26F is a heat map of the HI titer responses reports the corresponding percent similarity between the vaccine group and virus. Data is expressed as the mean with standard error (SEM). (****p<0.0001, ***p<0.001, **p<0.01; one-way ANOVA with Bonferroni multiple comparisons).

FIG. 27A-27D are graphs showing the antibody response after single shot vaccination with influenza strains A/CA/04/09 (FIG. 27A), A/PR/8/34 (FIG. 27B), A/NC/20/99 (FIG. 27C), and A/Brisbane/59/07 (FIG. 27D). The mean absorbance at OD450 is shown with standard error (SEM) (****p<0.0001, **p<0.01, *p<0.05, n.s. not significant; one-way ANOVA with Bonferroni multiple comparisons).

FIG. 28A-28D are graphs showing the total T cell response after single shot vaccination with A/California/07/09 (FIG. 28A), A/Puerto Rico/8/34 (FIG. 28B), A/New Caledonia/20/99 (FIG. 28C), or A/Brisbane/59/07 (FIG. 28D) strains. Data is expressed as the number of mean spot forming cells (SFC) per million splenocytes with standard error (SEM) (****p<0.0001, ****p<0.001, **p<0.01, *p<0.05; one-way ANOVA with Bonferroni multiple comparisons).

FIG. 29A is a schematic of positive responses (i.e., greater than 50 spot forming cells (SFC) per million splenocytes) shown in relation to the HA gene location.

FIG. 29B-29D are graphs showing the quantification of positive peptides for A/Brisbane/59/07 (FIG. 29B), A/New Caledonia/20/99 (FIG. 29C), and A/California/07/09 (FIG. 29D) shown schematically above the HA sequence to indicate the positions of possible epitopes. Data is expressed as the mean spot forming cells (SFC) per million splenocytes with standard error (SEM).

FIG. 30 are graphs that show the protection against A/California/04/09 lethal challenge. Weight loss for individual mice is shown in (FIG. 30A, FIG. 30B), average weight loss is shown in (FIG. 30C, FIG. 30D), and percent survival is shown in (FIG. 30E, FIG. 30F). (Survival data was analyzed with log rank test compared to the PBS control; ****p<0.0001, ***p<0.001, **p<0.01, *p<0.05).

FIG. 31 are graphs showing weight loss for individual mice (FIG. 31A, FIG. 31B), average weight loss (FIG. 31C, FIG. 31D), and percent survival (FIG. 31E, FIG. 31F) for mice immunized with either 10e8 or 10e10 virus particles, respectively, of recombinant Ad5-H1-Mosaic-HA, Ad5-A/PR/8/34-HA, Ad5-A/TX/05/09-HA (pdm09), 150 ng of Fluzone HA, or PBS and challenged with 100MLD50 3 weeks later. Survival data was analyzed with log rank test compared to the PBS control; *p<0.05.

FIG. 32 are graphs showing weight loss for individual mice (FIG. 32A, FIG. 32B), average weight loss (FIG. 32C, FIG. 32D), and percent survival (FIG. 32E, FIG. 32F) for mice immunized with either 10e8 or 10e10 virus particles, respectively, of recombinant Ad5-H1-Mosaic-HA, Ad5-A/PR/8/34-HA, Ad5-A/TX/05/09-HA (pdm09), 150 ng of Fluzone HA, or PBS and challenged with 100MLD50 3 weeks later. Survival data was analyzed with log rank test compared to the PBS control (**p<0.01, *p<0.05).

FIG. 33 are graphs that demonstrate protection against A/Nanchang/1/99 lethal challenge. Weight loss for individual mice is shown in (FIG. 33A, FIG. 33B), average weight loss is shown in (FIG. 33C, FIG. 33D) ****p<0.0001, ***p<0.001, **p<0.01, *p<0.05).

FIG. 34A shows a Jukes-Cantor neighbor joining tree constructed from an alignment of the Mosaic and three Epigraph immunogens described herein and the original sequence population.

FIG. 34B is a Western blot of Hemagglutinin expression from each vector. A wild type comparator HA gene (A/swine/Texas/4199-2/1998 [TX98]) was used as a control.

FIG. 35A shows the experimental time line for vaccination and boost.

FIG. 35B shows the representative divergent H3 swine influenza viruses that were chosen for analysis of antibody development.

FIG. 35C are graphs of hemmaglutination inhibition (HI) were performed as described by the World Health Organization standard protocols. HI titers are reported as geometric mean titers ±standard error. The dotted line represents the standard titer or 40 considered to confer protection against challenge.

FIG. 36A is a schematic showing the T cell epitopes induced by the vaccine immunogens to four representative divergent swH3 viruses. A peptide was considered positive if the spot-forming cells (SFC) per million was greater than 50.

FIG. 36B are graphs showing individual responses to positive peptides.

FIG. 36C are graphs showing total T-cell responses.

FIG. 37A are graphs showing the geometric mean HI titer was determined±standard error with representative human H3N2 virus isolates.

FIG. 37B is a schematic showing peptides considered positive for a cross-reactive T-cell immune responses against any of four representative human influenza strains.

FIG. 37C are graphs showing individual responses to positive peptides.

FIG. 37D are graphs showing total T-cell responses.

FIG. 38A are graphs showing weight loss in mice after challenge with Manitoba/2005.

FIG. 38B are graphs showing weight loss in mice after challenge with Colorado/1977.

### LIST OF SEQUENCES

SEQ ID NO:1 is the amino acid sequence of a universal HA vaccine from human H1 influenza A serotypes generated using consensus of unique sequence (COUS).

SEQ ID NO:2 is the amino acid sequence of a universal HA vaccine from human H1 (pH1N1) influenza A serotypes generated using a consensus of unique sequence (COUS).

SEQ ID NO:3 is the amino acid sequence of a universal HA vaccine from human H1 influenza A serotypes generated using Mosaic1.

SEQ ID NO:4 is the amino acid sequence of a universal HA vaccine from human H1 influenza A serotypes generated using Mosaic 2.

SEQ ID NO:5 is the amino acid sequence of a universal HA vaccine from human H1 influenza A serotypes generated using Mosaic 3.

SEQ ID NO:6 is the amino acid sequence of a universal HA vaccine from human H1 influenza A serotypes generated using Epigraph1.

SEQ ID NO:7 is the amino acid sequence of a universal HA vaccine from human H1 influenza A serotypes generated using Epigraph2.

SEQ ID NO:8 is the amino acid sequence of a universal HA vaccine from human H1 influenza A serotypes generated using Epigraph3.

SEQ ID NO:9 is the amino acid sequence of a universal HA vaccine from human H2 influenza A serotypes generated using COUS.

SEQ ID NO: 10 is the amino acid sequence of a universal HA vaccine from human H2 influenza A serotypes generated using Mosaic1.

SEQ ID NO:11 is the amino acid sequence of a universal HA vaccine from human H2 influenza A serotypes generated using Mosaic2.

SEQ ID NO: 12 is the amino acid sequence of a universal HA vaccine from human H2 influenza A serotypes generated using Mosaic3.

SEQ ID NO:13 is the amino acid sequence of a universal HA vaccine from human H2 influenza A serotypes generated using Epigraph1.

SEQ ID NO: 14 is the amino acid sequence of a universal HA vaccine from human H2 influenza A serotypes generated using Epigraph2.

SEQ ID NO:15 is the amino acid sequence of a universal HA vaccine from human H2 influenza A serotypes generated using Epigraph3.

SEQ ID NO:16 is the amino acid sequence of a universal HA vaccine from human H3 influenza A serotypes generated using COUS.

SEQ ID NO:17 is the amino acid sequence of a universal HA vaccine from human H3 influenza A serotypes generated using Mosaic1.

SEQ ID NO:18 is the amino acid sequence of a universal HA vaccine from human H3 influenza A serotypes generated using Mosaic2.

SEQ ID NO: 19 is the amino acid sequence of a universal HA vaccine from human H3 influenza A serotypes generated using Mosaic3.

SEQ ID NO:20 is the amino acid sequence of a universal HA vaccine from human H3 influenza A serotypes generated using Epigraph1.

SEQ ID NO:21 is the amino acid sequence of a universal HA vaccine from human H3 influenza A serotypes generated using Epigraph2.

SEQ ID NO:22 is the amino acid sequence of a universal HA vaccine from human H3 influenza A serotypes generated using Epigraph3.

SEQ ID NO:23 is the amino acid sequence of a universal HA vaccine from human H5 influenza A serotypes generated using COUS.

SEQ ID NO:24 is the amino acid sequence of a universal HA vaccine from human H5 influenza A serotypes generated using Mosaic1.

SEQ ID NO:25 is the amino acid sequence of a universal HA vaccine from human H5 influenza A serotypes generated using Mosaic2.

SEQ ID NO:26 is the amino acid sequence of a universal HA vaccine from human H5 influenza A serotypes generated using Mosaic3.

SEQ ID NO:27 is the amino acid sequence of a universal HA vaccine from human H5 influenza A serotypes generated using Epigraph1.

SEQ ID NO:28 is the amino acid sequence of a universal HA vaccine from human H5 influenza A serotypes generated using Epigraph2.

SEQ ID NO:29 is the amino acid sequence of a universal HA vaccine from human H5 influenza A serotypes generated using Epigraph3.

SEQ ID NO:30 is the amino acid sequence of a universal NA vaccine from human N1 influenza A serotypes generated using COUS.

SEQ ID NO:31 is the amino acid sequence of a universal NA vaccine from human N1 influenza A serotypes generated using Mosaic1.

SEQ ID NO:32 is the amino acid sequence of a universal NA vaccine from human N1 influenza A serotypes generated using Mosaic2.

SEQ ID NO:33 is the amino acid sequence of a universal NA vaccine from human N1 influenza A serotypes generated using Mosaic3.

SEQ ID NO:34 is the amino acid sequence of a universal NA vaccine from human N1 influenza A serotypes generated using Epigraph1.

SEQ ID NO:35 is the amino acid sequence of a universal NA vaccine from human N1 influenza A serotypes generated using Epigraph2.

SEQ ID NO:36 is the amino acid sequence of a universal NA vaccine from human N1 influenza A serotypes generated using Epigraph3.

SEQ ID NO:37 is the amino acid sequence of a universal NA vaccine from human N2 influenza A serotypes generated using COUS.

SEQ ID NO:38 is the amino acid sequence of a universal NA vaccine from human N2 influenza A serotypes generated using Mosaic1.

SEQ ID NO:39 is the amino acid sequence of a universal NA vaccine from human N2 influenza A serotypes generated using Mosaic2.

SEQ ID NO:40 is the amino acid sequence of a universal NA vaccine from human N2 influenza A serotypes generated using Mosaic3.

SEQ ID NO:41 is the amino acid sequence of a universal NA vaccine from human N2 influenza A serotypes generated using Epigraph1.

SEQ ID NO:42 is the amino acid sequence of a universal NA vaccine from human N2 influenza A serotypes generated using Epigraph2.

SEQ ID NO:43 is the amino acid sequence of a universal NA vaccine from human N2 influenza A serotypes generated using Epigraph3.

SEQ ID NO:44 is the amino acid sequence of a universal HA vaccine from human Victoria-like influenza B serotypes generated using COUS.

SEQ ID NO:45 is the amino acid sequence of a universal HA vaccine from human Victoria-like influenza B serotypes generated using Centered Consensus (CC).

SEQ ID NO:46 is the amino acid sequence of a universal HA vaccine from human Victoria-like influenza B serotypes generated using Mosaic1.

SEQ ID NO:47 is the amino acid sequence of a universal HA vaccine from human Victoria-like influenza B serotypes generated using Mosaic2.

SEQ ID NO:48 is the amino acid sequence of a universal HA vaccine from human Victoria-like influenza B serotypes generated using Mosaic3.

SEQ ID NO:49 is the amino acid sequence of a universal HA vaccine from human Victoria-like influenza B serotypes generated using Epigraph1.

SEQ ID NO:50 is the amino acid sequence of a universal HA vaccine from human Victoria-like influenza B serotypes generated using Epigraph2.

SEQ ID NO:51 is the amino acid sequence of a universal HA vaccine from human Victoria-like influenza B serotypes generated using Epigraph3.

SEQ ID NO:52 is the amino acid sequence of a universal HA vaccine from human Victoria-like influenza B serotypes generated using Epigraph4.

SEQ ID NO:53 is the amino acid sequence of a universal HA vaccine from human Yamagata-like influenza B serotypes generated using COUS.

SEQ ID NO:54 is the amino acid sequence of a universal HA vaccine from human Yamagata-like influenza B serotypes generated using Mosaic1.

SEQ ID NO:55 is the amino acid sequence of a universal HA vaccine from human Yamagata-like influenza B serotypes generated using Mosaic2.

SEQ ID NO:56 is the amino acid sequence of a universal HA vaccine from human Yamagata-like influenza B serotypes generated using Mosaic3.

SEQ ID NO:57 is the amino acid sequence of a universal HA vaccine from human Yamagata-like influenza B serotypes generated using Epigraph1.

SEQ ID NO:58 is the amino acid sequence of a universal HA vaccine from human Yamagata-like influenza B serotypes generated using Epigraph2.

SEQ ID NO:59 is the amino acid sequence of a universal HA vaccine from human Yamagata-like influenza B serotypes generated using Epigraph3.

SEQ ID NO:60 is the amino acid sequence of a universal HA vaccine from human Yamagata-like influenza B serotypes generated using Epigraph4.

SEQ ID NO:61 is the amino acid sequence of a universal NA vaccine from human Victoria-like influenza B serotypes generated using COUS.

SEQ ID NO:62 is the amino acid sequence of a universal NA vaccine from human Victoria-like influenza B serotypes generated using Mosaic1.

SEQ ID NO:63 is the amino acid sequence of a universal NA vaccine from human Victoria-like influenza B serotypes generated using Mosaic2.

SEQ ID NO:64 is the amino acid sequence of a universal NA vaccine from human Victoria-like influenza B serotypes generated using Mosaic3.

SEQ ID NO:65 is the amino acid sequence of a universal NA vaccine from human Victoria-like influenza B serotypes generated using Epigraph1.

SEQ ID NO:66 is the amino acid sequence of a universal NA vaccine from human Victoria-like influenza B serotypes generated using Epigraph2.

SEQ ID NO:67 is the amino acid sequence of a universal NA vaccine from human Victoria-like influenza B serotypes generated using Epigraph3.

SEQ ID NO:68 is the amino acid sequence of a universal NA vaccine from human Yamagata-like influenza B serotypes generated using COUS.

SEQ ID NO:69 is the amino acid sequence of a universal NA vaccine from human Yamagata-like influenza B serotypes generated using Mosaic1.

SEQ ID NO:70 is the amino acid sequence of a universal NA vaccine from human Yamagata-like influenza B serotypes generated using Mosaic2.

SEQ ID NO:71 is the amino acid sequence of a universal NA vaccine from human Yamagata-like influenza B serotypes generated using Mosaic3.

SEQ ID NO:72 is the amino acid sequence of a universal NA vaccine from human Yamagata-like influenza B serotypes generated using Epigraph1.

SEQ ID NO:73 is the amino acid sequence of a universal NA vaccine from human Yamagata-like influenza B serotypes generated using Epigraph2.

SEQ ID NO:74 is the amino acid sequence of a universal NA vaccine from human Yamagata-like influenza B serotypes generated using Epigraph3.

SEQ ID NO:75 is the amino acid sequence of a universal HA vaccine from swine H1 influenza A serotypes using COUS.

SEQ ID NO:76 is the amino acid sequence of a universal HA vaccine from swine H1 influenza A serotypes using Mosaic1.

SEQ ID NO:77 is the amino acid sequence of a universal HA vaccine from swine H1 influenza A serotypes using Mosaic2.

SEQ ID NO:78 is the amino acid sequence of a universal HA vaccine from swine H1 influenza A serotypes using Mosaic3.

SEQ ID NO:79 is the amino acid sequence of a universal HA vaccine from swine H1 influenza A serotypes using Epigraph1.

SEQ ID NO:80 is the amino acid sequence of a universal HA vaccine from swine H1 influenza A serotypes using Epigraph2.

SEQ ID NO:81 is the amino acid sequence of a universal HA vaccine from swine H1 influenza A serotypes using Epigraph3.

SEQ ID NO:82 is the amino acid sequence of a universal HA vaccine from swine H3 influenza A serotypes using COUS.

SEQ ID NO:83 is the amino acid sequence of a universal HA vaccine from swine H3 influenza A serotypes using Mosaic1.

SEQ ID NO:84 is the amino acid sequence of a universal HA vaccine from swine H3 influenza A serotypes using Mosaic2.

SEQ ID NO:85 is the amino acid sequence of a universal HA vaccine from swine H3 influenza A serotypes using Mosaic3.

SEQ ID NO:86 is the amino acid sequence of a universal HA vaccine from swine H3 influenza A serotypes using Epigraph1.

SEQ ID NO:87 is the amino acid sequence of a universal HA vaccine from swine H3 influenza A serotypes using Epigraph2.

SEQ ID NO:88 is the amino acid sequence of a universal HA vaccine from swine H3 influenza A serotypes using Epigraph3.

SEQ ID NO:89 is the amino acid sequence of a universal NA vaccine from swine N1 influenza A serotypes using COUS.

SEQ ID NO:90 is the amino acid sequence of a universal NA vaccine from swine N1 influenza A serotypes using Mosaic1.

SEQ ID NO:91 is the amino acid sequence of a universal NA vaccine from swine N1 influenza A serotypes using Mosaic2.

SEQ ID NO:92 is the amino acid sequence of a universal NA vaccine from swine N1 influenza A serotypes using Mosaic3.

SEQ ID NO:93 is the amino acid sequence of a universal NA vaccine from swine N1 influenza A serotypes using Epigraph1.

SEQ ID NO:94 is the amino acid sequence of a universal NA vaccine from swine N1 influenza A serotypes using Epigraph2.

SEQ ID NO:95 is the amino acid sequence of a universal NA vaccine from swine N1 influenza A serotypes using Epigraph3.

SEQ ID NO:96 is the amino acid sequence of a universal NA vaccine from swine N2 influenza A serotypes using COUS.

SEQ ID NO:97 is the amino acid sequence of a universal NA vaccine from swine N2 influenza A serotypes using Mosaic1.

SEQ ID NO:98 is the amino acid sequence of a universal NA vaccine from swine N2 influenza A serotypes using Mosaic2.

SEQ ID NO:99 is the amino acid sequence of a universal NA vaccine from swine N2 influenza A serotypes using Mosaic3.

SEQ ID NO:100 is the amino acid sequence of a universal NA vaccine from swine N2 influenza A serotypes using Epigraph1.

SEQ ID NO:101 is the amino acid sequence of a universal NA vaccine from swine N2 influenza A serotypes using Epigraph2.

SEQ ID NO: 102 is the amino acid sequence of a universal NA vaccine from swine N2 influenza A serotypes using Epigraph3.

SEQ ID NO:103 is a nucleic acid sequence encoding a universal HA vaccine from human

H1 influenza A using Consensus (Cons).

SEQ ID NO:104 is a nucleic acid sequence encoding a universal HA vaccine from human H1 influenza A using Mosaic1.

SEQ ID NO:105 is a nucleic acid sequence encoding a universal HA vaccine from human H1 influenza A using Epigraph1.

SEQ ID NO:106 is a nucleic acid sequence encoding a universal HA vaccine from human H1 influenza A using Epigraph2.

SEQ ID NO:107 is a nucleic acid sequence encoding a universal HA vaccine from human H1 influenza A using Epigraph3.

SEQ ID NO:108 is a nucleic acid sequence encoding a universal HA vaccine from human H3 influenza A using Consensus (Cons).

SEQ ID NO:109 is a nucleic acid sequence encoding a universal HA vaccine from human H3 influenza A using Mosaic1.

SEQ ID NO:110 is a nucleic acid sequence encoding a universal HA vaccine from human H3 influenza A using Epigraph1.

SEQ ID NO: 111 is a nucleic acid sequence encoding a universal HA vaccine from human H3 influenza A using Epigraph2.

SEQ ID NO:112 is a nucleic acid sequence encoding a universal HA vaccine from human H3 influenza A using Epigraph3.

SEQ ID NO:113 is a nucleic acid sequence encoding a universal HA vaccine from swine

H1 influenza A using Consensus.

SEQ ID NO:114 is a nucleic acid sequence encoding a universal HA vaccine from swine H1 influenza A using Mosaic1.

SEQ ID NO:115 is a nucleic acid sequence encoding a universal HA vaccine from swine H1 influenza A using Epigraph1.

SEQ ID NO:116 is a nucleic acid sequence encoding a universal HA vaccine from swine

H1 influenza A using Epigraph2.

SEQ ID NO:117 is a nucleic acid sequence encoding a universal HA vaccine from swine

H1 influenza A using Epigraph3.

SEQ ID NO: 118 is a nucleic acid sequence encoding a universal NA vaccine from swine

H3 influenza A using Consensus.

SEQ ID NO: 119 is a nucleic acid sequence encoding a universal NA vaccine from swine H3 influenza A using Mosaic1.

SEQ ID NO:120 is a nucleic acid sequence encoding a universal NA vaccine from swine H3 influenza A using Epigraph1.

SEQ ID NO:121 is a nucleic acid sequence encoding a universal NA vaccine from swine H3 influenza A using Epigraph2.

SEQ ID NO:122 is a nucleic acid sequence encoding a universal NA vaccine from swine H3 influenza A using Epigraph3.

### DETAILED DESCRIPTION

A universal vaccine for influenza has become a high priority and there are several efforts being pursued to achieve this goal, since prevention of influenza in humans would have a very significant impact on healthcare costs and burden. Here, a solution is proposed to create universal virus vaccines using several strategies that have not been previously employed. Therefore, the approach described herein is unique because it utilized all known strains infecting humans and swine, and thus, the genes described herein are unique and do not exist in nature.

Trivalent Inactivated Vaccine (TIV) (e.g., FluZone) is the workhorse of modern influenza vaccination and has protected millions of humans from influenza morbidity and mortality. While effective, TIV has limitations that support the development of alternate vaccine platforms. For example, production of the vaccine in embryonated eggs is one fundamental problem with this vaccine platform. A large fraction of humans are allergic to egg ovalbumin and cannot take this vaccine. Generating influenza vaccines in eggs is time consuming, labor intensive, and relies on the availability of embryonated eggs. TIV only provides short-term immunity and this immunity is highly strain specific. Intramuscular delivery does not stimulate high levels of the secretory IgA that is thought to be more reactive against heterologous viruses, and TIV also importantly fails to induce cross-protective T cell immunity.

Live-Attenuated Cold-Adapted Vaccines (e.g., FluMist) is a cold-adapted influenza virus platform that can abortively replicate when introduced intranasally where the temperature is below 37° C. For this reason, FluMist can be superior to TIV because it can drive a combination of antibody and T cell responses. Cold adaptation has the advantage of faster vaccine production, induces protection, and has the ability to generate both mucosal and humoral immune responses that include secretory IgA, serum IgG, cytokine stimulation and the priming of CTL responses. The platform is limited by short duration of immunity, strain specificity, and contraindicated use in the very young, elderly, or immunocompromised.

Influenza surveillance in Asia and Australia is used to predict which influenza strains might spread in the United States in the following year. This generally effective strategy can fail. In fact, the concordance of WHO recommended strains and those strains causing outbreaks was only 50% between 1997 and 2005. Put another way, during those influenza seasons the strain predictions failed 50% of the time, leaving humans unprotected against seasonal and potentially pandemic influenza. The degree of vaccine mismatch with the 2009 pandemic Swine flu, based on HA amino acid divergence (20.5%), approached the maximal ˜21% HA amino acid divergence between all H1 strains. As a result of this mismatch, the 2009 pandemic infected 24% of the global population. In contrast, the HA1-COT immunogen is only 7.6% divergent from the pandemic strain and provides protection in mouse challenge models.

The vaccine strategies described herein will be important to the prevention of influenza and will significantly reduce or eliminate the probability of emergent zoonotic re-assorted influenza viruses from swine.

Centralized vaccine genes have been shown to induce broader cross-reactive immunity against divergent virus strains as compared to wild type genes and may be very useful as universal vaccines. These centralized vaccine genes can be constructed by several different methods. Vaccine genes designed using the Consensus, Mosaic and Epigraph methods were evaluated herein for their ability to induce protection against divergent H1N1 and H3N2 Swine Influenza A Virus as universal vaccine genes.

The following methodologies were used to create the universal vaccine immunogens described herein:

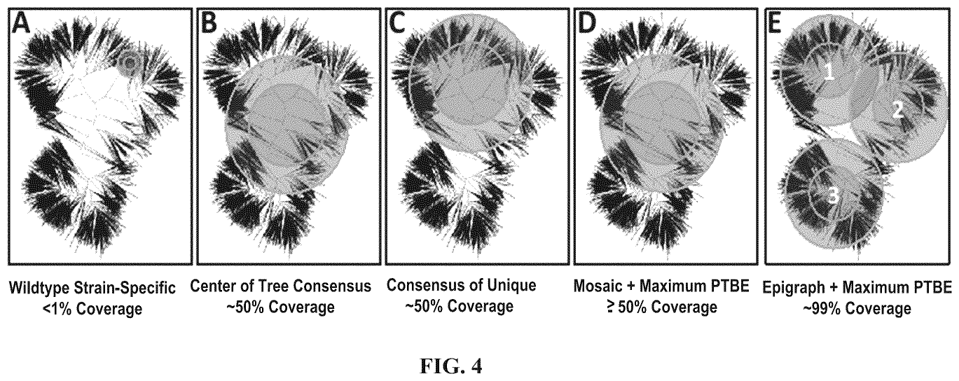

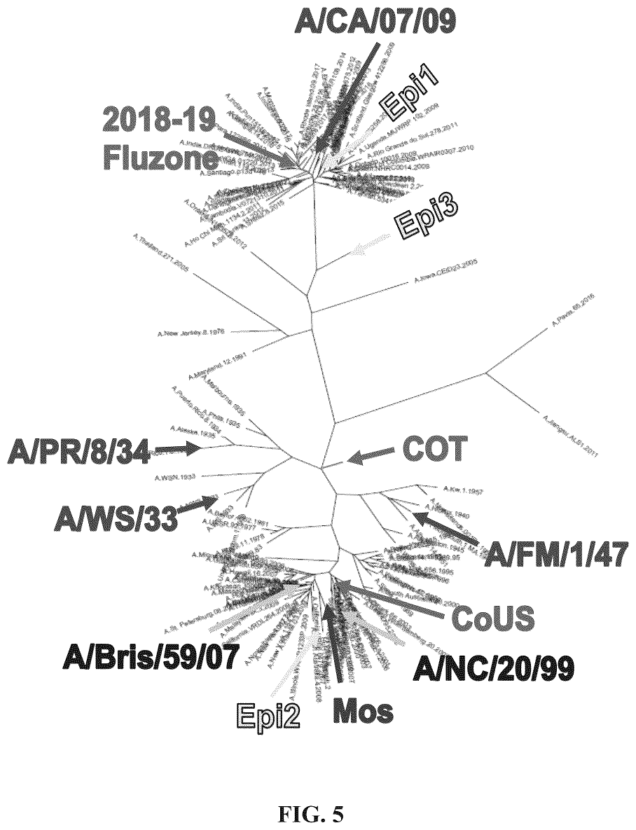

Centralized Consensus (CC) Genes-A CC gene is constructed using the most common amino acid at each position in a protein using an alignment of representative wild type gene sequences. This strategy creates a vaccine gene that localizes to the center of the phylogenetic tree and is equidistant to all known wild type virus strains. See FIG. 4B. Since functional elements, such as cleavage sites, transmembrane domains, localization signals, and fusion domains are critical to natural proteins, they are preserved within the CC genes. This technique has been shown to induce greater cross-reactive immunity to divergent HIV and Influenza viruses compared to wild type genes.

Consensus of Unique Sequences (COUS) Genes-A COUS gene is similar to a CC gene in that it is also uses the most common amino acid at each position. However, the alignment used to create the COUS gene consists of all known unique sequences for the given gene. This technique accounts for all of the known variants in the database and is biased towards the majority. FIG. 4C. The greatest advantage of this technique is that it selects a vaccine gene that is most closely related to the predominant circulating strains and, therefore, more likely to be protective against a randomly selected wild type challenge strain.

Mosaic Genes—In silico recombination is used to generate Mosaic genes. Full-length natural sequences are repetitively recombined with preference given to 9-mers duplicated in the sequences. This strategy uses a genetic algorithm to select for the highest possible potential T cell epitopes (PTE). FIG. 4D. Mosaic genes have shown to be promising vaccine candidates for HIV and other pathogens including HCV, Ebola and Influenza. Fischer et al., 2007, Nature Med., 13:100-6.

Epigraph Genes-Mosaic and Epigraph genes are designed to offer the same optimization of PTEs, however Epigraph genes provide substantially greater advantages over the Mosaic and consensus approaches. Unlike Mosaic approaches that use an algorithm, Epigraphs are designed using a faster graph-based design. A major element to the Epigraph approach is the ability to design multiple Epigraph genes that maximize vaccine coverage. FIG. 4E. The multiple Epigraph genes can localize to the major phylogenetic clusters of natural proteins or can span coverage across multiple clusters using recombinant Epigraph genes. This approach has been used to develop personalized therapeutic HIV vaccine candidates and a Pan-Filovirus Vaccine. Theiler and Korber, 2018, Stat. Med., 37:181-94.

It is proposed that centralized antigens will induce the greatest degree of cross-protective immune responses against mismatched influenza challenge, and, if so, they will impart a large impact on the field of influenza vaccine research. Examples of changes to the clinical practice of influenza vaccination may include the use of centralized antigens in the annual trivalent injection vaccine (TIV) or the stockpiling of centralized vaccines for use in the case of vaccine mismatch. Repeated exposure to a centralized vaccine, if used, for example, in an annual formulation, may induce enhanced cross-protective responses due to repeated boosting and affinity maturation to conserved epitopes in the centralized antigens.

Our approach to creating Centralized Consensus influenza genes is unique. For the consensus HA antigens, unique sequences were identified in the database. This eliminates repetitive sequences that may bias the consensus antigens towards more common strains and allows the reconstruction of a centralized sequence that represents all of the evolved sequences equally. Based on preliminary data in mice, the inclusion of a centralized gene in a swine influenza vaccine would reduce the risk of complete vaccine mismatch. Additionally, the centralized gene may boost responses against conserved elements that are common to all strains of influenza within that subtype.

The COUS genes that are described herein also are unique in that they are created using a dataset that consists of all known and unique influenza genes. This strategy biases these genes toward the predominant circulating strains. Therefore, the genetic relationship of the COUS genes and the circulating viruses is optimized. This strategy increases the probability of vaccine efficacy.

In order to create the Mosaic swine influenza vaccine antigens, the Mosaic Vaccine Designer program from the Los Alamos National Laboratories Tool Suite was used. Again, unique swine influenza HA protein sequences were obtained from the Influenza Research Database and were computationally analyzed for optimal potential T cell epitope coverage.

Lastly, the same HA data set was submitted to the Epigraph software suite where a three HA antigen cocktail was created. The Epigraph software selects for the immunogen that best represents the most common B and T cell epitopes in the dataset. This immunogen is designated Epigraph1. A second immunogen is designed to represent the second most common B and T cell epitopes, referred to as Epigraph2. Finally, a third immunogen representing the third most common B and T cell epitopes is designed and designated Epigraph3.

All the vaccine antigens were analyzed phylogenetically and found to localize either to the center of the phylogenetic tree, in the case of Centralized Consensus and Mosaic proteins, or to the two main influenza clusters with a recombinant covering the minor cluster, in the case of the Epigraph vaccines (FIG. 4).

The methodologies described herein can be applied to other viruses with high levels of genetic diversity (e.g., human immunodeficiency virus (HIV), Newcastle disease virus (NDV), porcine reproductive and respiratory syndrome (PRRS), or human papillomavirus (HPV)). Although the centralized influenza genes described herein can be used as human vaccines, these same strategies can be applied to vaccinate reservoir animals (e.g., swine, poultry, horses) against influenza infections and significantly reduce or eliminate the probability of the emergence of re-assorted swine influenza viruses. Thus, the vaccination of reservoir animals at the source of virus evolution could intervene at this primary step and result in the elimination of potential future zoonotic influenza outbreaks that result in pandemics.

A number of different vaccine polypeptides were generated; the amino acid sequences of such polypeptides are shown in SEQ ID NO:1-102 and representative nucleic acid sequences are shown in SEQ ID NO: 103-122. Those skilled in the art would appreciate that a nucleic acid sequence encoding each amino acid sequence can readily be determined.

In addition to the vaccine polypeptides having the sequences shown in SEQ ID NOs: 1-102 and encoded by the nucleic acids shown in SEQ ID NOs: 103-122, polypeptides and nucleic acids are provided that have at least 90% sequence identity (e.g., at least 91%, at least 92%, at least 93%, at least 94%, at least 95%, at least 96%, at least 97%, at least 98%, at least 99% or 100% sequence identity) to the vaccine polypeptides having the sequences shown in SEQ ID NOs: 1-102 or to the nucleic acids shown in SEQ ID NOs: 103-122.

In calculating percent sequence identity, two sequences are aligned and the number of identical matches of nucleotides or amino acid residues between the two sequences is determined. The number of identical matches is divided by the length of the aligned region (i.e., the number of aligned nucleotides or amino acid residues) and multiplied by 100 to arrive at a percent sequence identity value. It will be appreciated that the length of the aligned region can be a portion of one or both sequences up to the full-length size of the shortest sequence. It also will be appreciated that a single sequence can align with more than one other sequence and hence, can have different percent sequence identity values over each aligned region.

The alignment of two or more sequences to determine percent sequence identity can be performed using the algorithm described by Altschul et al. (1997, Nucleic Acids Res., 25:3389 3402) as incorporated into BLAST (basic local alignment search tool) programs, available at ncbi.nlm.nih.gov on the World Wide Web. BLAST searches can be performed to determine percent sequence identity between a sequence (nucleic acid or amino acid) and any other sequence or portion thereof aligned using the Altschul et al. algorithm. BLASTN is the program used to align and compare the identity between nucleic acid sequences, while BLASTP is the program used to align and compare the identity between amino acid sequences. When utilizing BLAST programs to calculate the percent identity between a sequence and another sequence, the default parameters of the respective programs generally are used.

Vectors containing nucleic acid molecules that encode polypeptides also are provided. Vectors, including expression vectors, are commercially available or can be produced by recombinant technology. A vector containing a nucleic acid molecule can have one or more elements for expression operably linked to such a nucleic acid molecule, and further can include sequences such as those encoding a selectable marker (e.g., an antibiotic resistance gene), and/or those that can be used in purification of a polypeptide (e.g., 6×His tag). Elements for expression include nucleic acid sequences that direct and regulate expression of nucleic acid coding sequences. One example of an expression element is a promoter sequence. Expression elements also can include one or more of introns, enhancer sequences, response elements, or inducible elements that modulate expression of a nucleic acid molecule. Expression elements can be of bacterial, yeast, insect, mammalian, or viral origin and vectors can contain a combination of expression elements from different origins. As used herein, operably linked means that elements for expression are positioned in a vector relative to a coding sequence in such a way as to direct or regulate expression of the coding sequence.

A nucleic acid molecule, e.g., a nucleic acid molecule in a vector (e.g., an expression vector, a viral vector) can be introduced into a host cell. The term “host cell” refers not only to the particular cell(s) into which the nucleic acid molecule has been introduced, but also to the progeny or potential progeny of such a cell. Many suitable host cells are known to those skilled in the art; host cells can be prokaryotic cells (e.g., E. coli) or eukaryotic cells (e.g., yeast cells, insect cells, plant cells, mammalian cells). Representative host cells can include, without limitation, A549, WEHI, 3T3, 10T1/2, BHK, MDCK, COS 1, COS 7, BSC 1, BSC 40, BMT 10, VERO, WI38, HeLa, 293 cells, Saos, C2C12, L cells, HT1080, HepG2 and primary fibroblast, hepatocyte and myoblast cells derived from mammals including human, monkey, mouse, rat, rabbit, and hamster. Methods for introducing nucleic acid molecules into host cells are well known in the art and include, without limitation, calcium phosphate precipitation, electroporation, heat shock, lipofection, microinjection, and viral-mediated nucleic acid transfer (e.g., transduction).

With respect to polypeptides, “purified” refers to a polypeptide (i.e., a peptide or a polypeptide) that has been separated or purified from cellular components that naturally accompany it. Typically, the polypeptide is considered “purified” when it is at least 70% (e.g., at least 75%, 80%, 85%, 90%, 95%, or 99%) by dry weight, free from the polypeptides and naturally occurring molecules with which it is naturally associated. Since a polypeptide that is chemically synthesized is, by nature, separated from the components that naturally accompany it, a synthetic polypeptide is considered “purified,” but further can be removed from the components used to synthesize the polypeptide (e.g., amino acid residues). With respect to nucleic acid molecules, “isolated” refers to a nucleic acid molecule that is separated from other nucleic acid molecules that are usually associated with it in the genome. In addition, an isolated nucleic acid molecule can include an engineered nucleic acid molecule such as a recombinant or a synthetic nucleic acid molecule.

Polypeptides can be obtained (e.g., purified) from natural sources (e.g., a biological sample) by known methods such as DEAE ion exchange, gel filtration, and/or hydroxyapatite chromatography. A purified polypeptide also can be obtained, for example, by expressing a nucleic acid molecule in an expression vector or by chemical synthesis. The extent of purity of a polypeptide can be measured using any appropriate method, e.g., column chromatography, polyacrylamide gel electrophoresis, or HPLC analysis. Similarly, nucleic acid molecules can be obtained (e.g., isolated) using routine methods such as, without limitation, recombinant nucleic acid technology (e.g., restriction enzyme digestion and ligation) or the polymerase chain reaction (PCR; see, for example, PCR Primer: A Laboratory Manual, Dieffenbach & Dveksler, Eds., Cold Spring Harbor Laboratory Press, 1995). In addition, isolated nucleic acid molecules can be chemically synthesized.

In some embodiments, a vaccine as described herein can be multivalent (e.g., 4-valent, 12-valent, 16-valent). For example, a vaccine can include a plurality of polypeptides as described herein. For example, a vaccine can include one or more of the Mosaic1 vaccine polypeptides (e.g., SEQ ID NOs: 3, 10, 17, 24, 31, 38, 46, 54, 62, 69, 76, 83, 90, 97, 104, 109, 114, or 119), one or more of the Epigraph1 vaccine polypeptides (e.g., SEQ ID NOs: 6, 13, 20, 27, 34, 41, 49, 57, 65, 72, 79, 86, 93, 100, 105, 110, 115, or 120), one or more of the Mosaic2 vaccine polypeptides (e.g., SEQ ID NOs: 4, 11, 18, 25, 32, 39, 47, 55, 63, 70, 77, 84, 91, or 98), one or more of the Epigraph2 vaccine polypeptides (SEQ ID NOs: 7, 14, 21, 28, 35, 42, 50, 58, 66, 73, 80, 87, 94, 101, 106, 111, 116, or 121), or combinations thereof. In some embodiments, a vaccine can include one or more of the Epigraph vaccine polypeptides (e.g., Epigraph1, Epigraph2, Epigraph3; e.g., SEQ ID NOs: 6-8, 13-15, 20-22, 27-29, 34-36, 41-43, 49-52, 57-60, 65-67, 72-74, 79-81, 86-88, 93-95, or 100-102).

Typically, one or more of the human vaccine polypeptides described herein are administered to a human to provide protective immunity against influenza and one or more of the swine vaccine polypeptides described herein are administered to a swine to provide protective immunity against influenza. In some instances, however, one or more of the human vaccine polypeptides described herein can be administered to an animal (e.g., a bird, a pig, a horse, a dog, a cat) to provide protective immunity against influenza (e.g., avian influenza, swine influenza, equine influenza, canine influenza, feline influenza), and/or one or more of the swine vaccine polypeptides described herein can be administered to a human or a non-swine animal to provide protective immunity against influenza.

The vaccine polypeptides described herein can be suspended in a physiologically acceptable or compatible carrier and administered to a subject. Pharmaceutically acceptable carriers include saline, which may be formulated with a variety of buffering solutions (e.g., phosphate buffered saline), lactose, sucrose, calcium phosphate, gelatin, dextran, agar, pectin, and water. Conventional and pharmaceutically acceptable routes of administration include, but are not limited to, direct delivery to an organ such as, for example, the liver or lung, orally, intranasally, intratracheally, by inhalation, intravenously, intramuscularly, intraocularly, subcutaneously, intradermally, transmucosally, or by other routes of administration. The vaccine polypeptides described herein, or nucleic acids (e.g., DNA, RNA) encoding such viral polypeptides, can be delivered using a viral vector (e.g., an adenovirus, an adeno-associated virus, a retrovirus, an alphavirus, a paramyxovirus, or a rhabdovirus) or nanoparticles (e.g., lipid nanoparticles).

The vaccine polypeptides described herein can be administered in sufficient amounts to provide a therapeutic benefit without undue adverse effects. The dose of the vaccine polypeptide administered to a subject will depend primarily on factors such as the condition being treated, and the age, weight, and health of the subject. Simply by way of example, an effective dosage of a vaccine polypeptide to be administered to a human subject can be in the range of from about 15 micrograms (μg) to about 90 μg (e.g., about 20 μg to about 80 μg; about 25 μg to about 75 μg; about 30 μg to about 70 μg; about 40 μg to about 60 μg; about 45 μg to about 55 μg; about 50 μg). In some instances, a subject can be administered one or more of the vaccine polypeptides described herein seasonally or upon detection of influenza or antibodies against influenza; in some instances, a subject can be administered one or more of the vaccine polypeptides described herein in early childhood, thereby providing protective immunity in that subject for years or decades. Under certain circumstances, it may be desirable to re-administer one or more of the vaccine polypeptides described herein to the subject after a period of time following the first administration (e.g., weeks, months, years (e.g., decades)) in the form or a “booster” vaccine.

In accordance with the present invention, there may be employed conventional molecular biology, microbiology, biochemical, and recombinant DNA techniques within the skill of the art. Such techniques are explained fully in the literature. The invention will be further described in the following examples, which do not limit the scope of the methods and compositions of matter described in the claims.

### Examples

### Example 1—Viral Genes and the Generated Vaccine Sequences

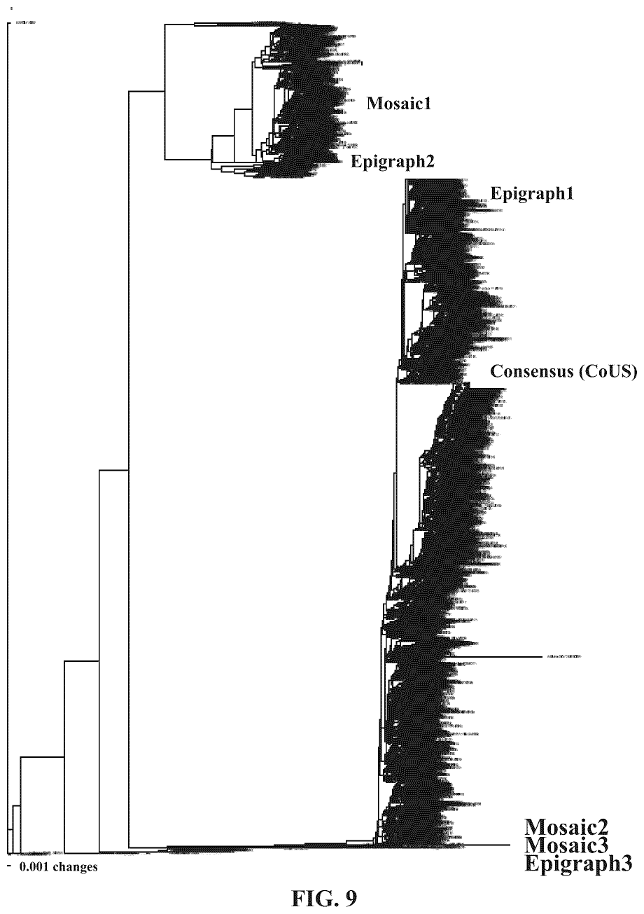

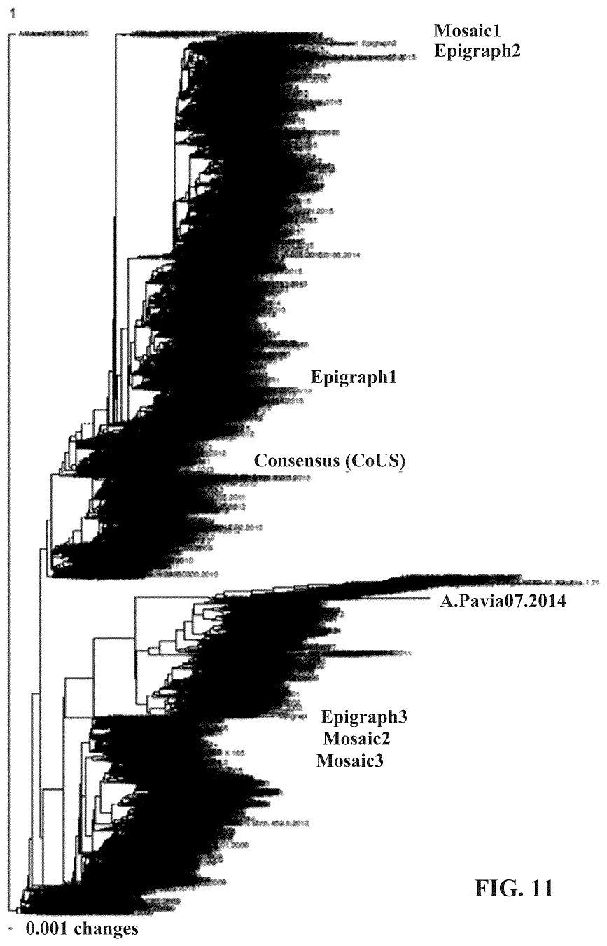

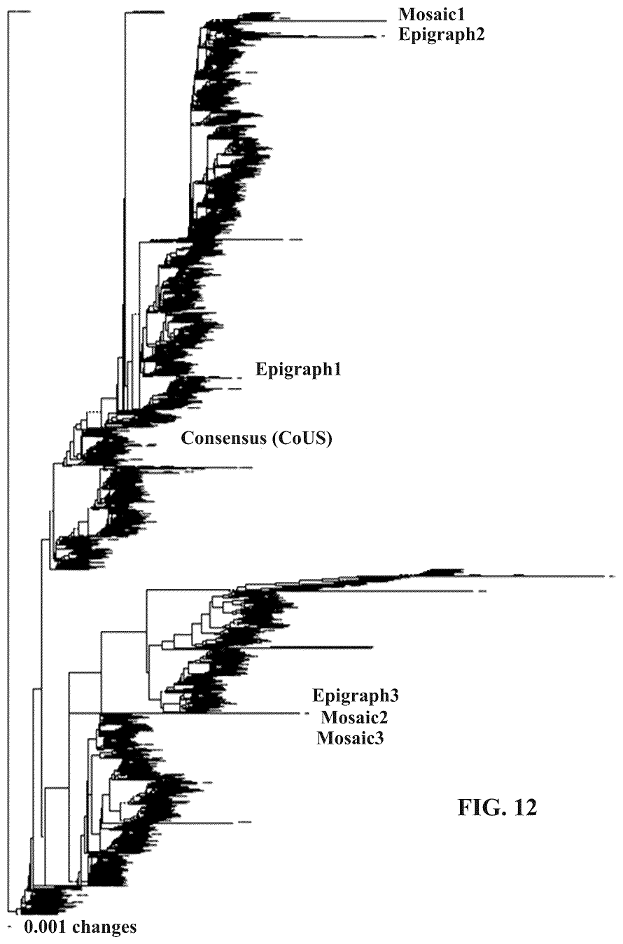

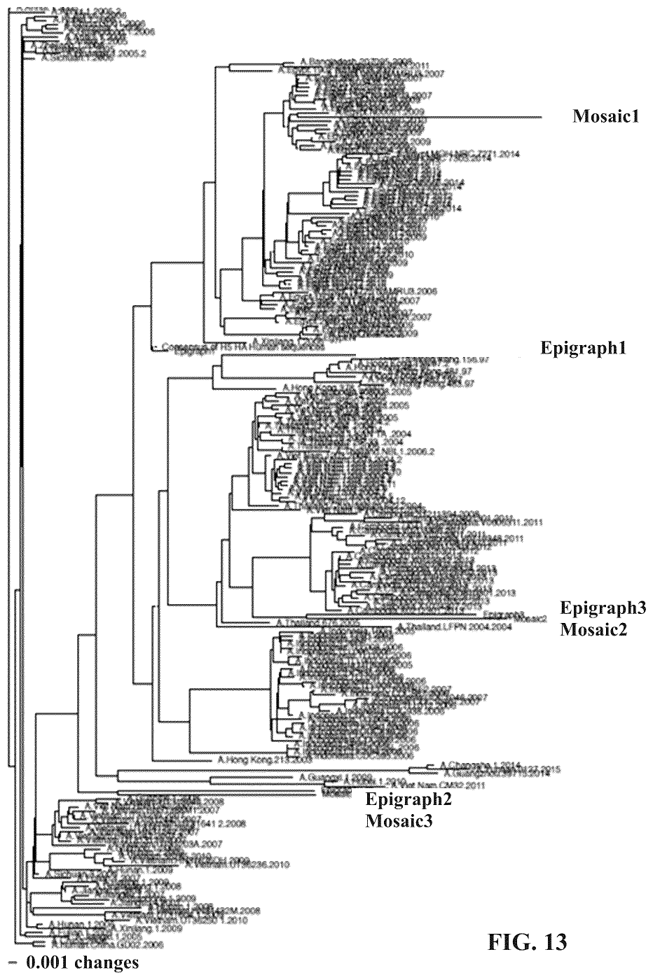

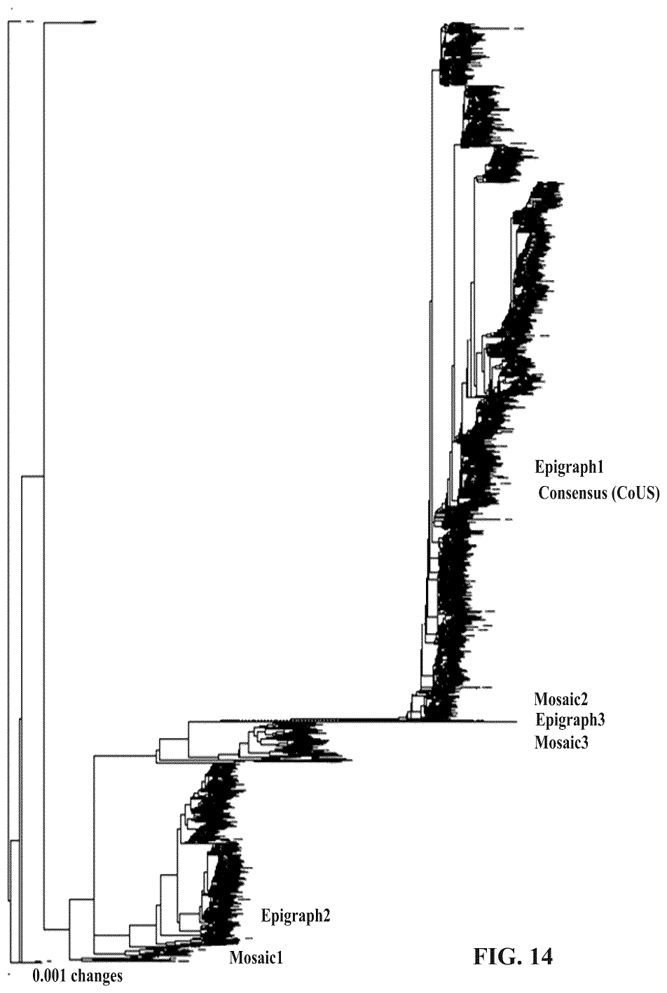

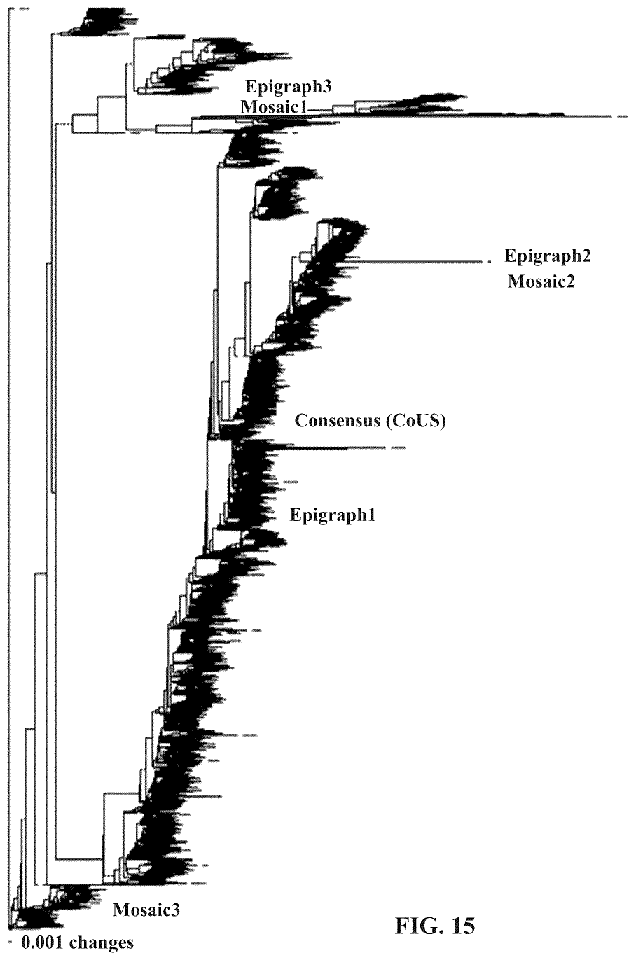

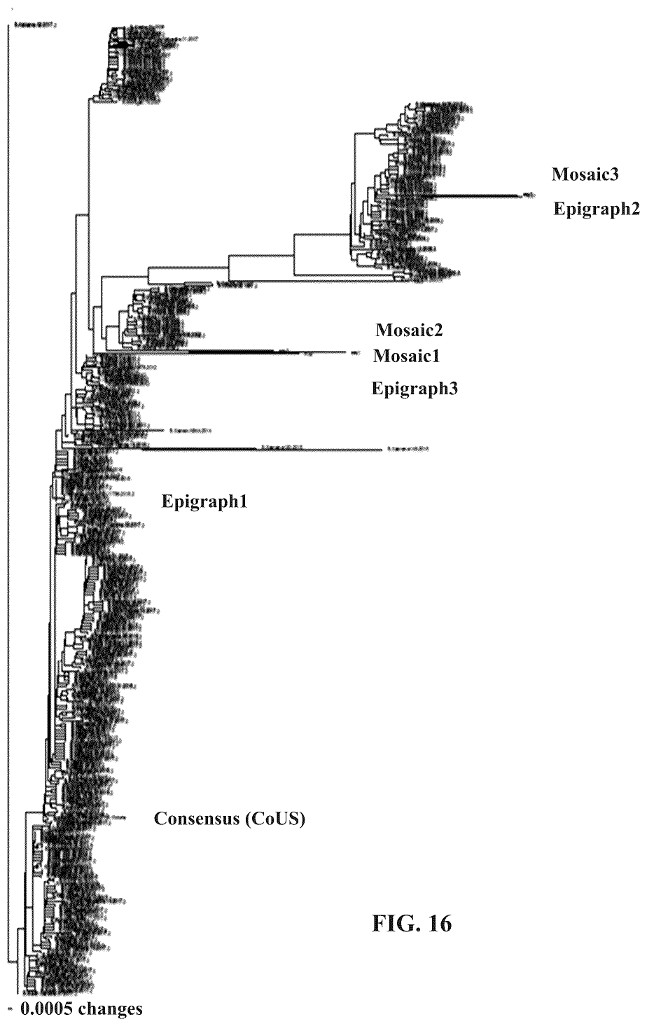

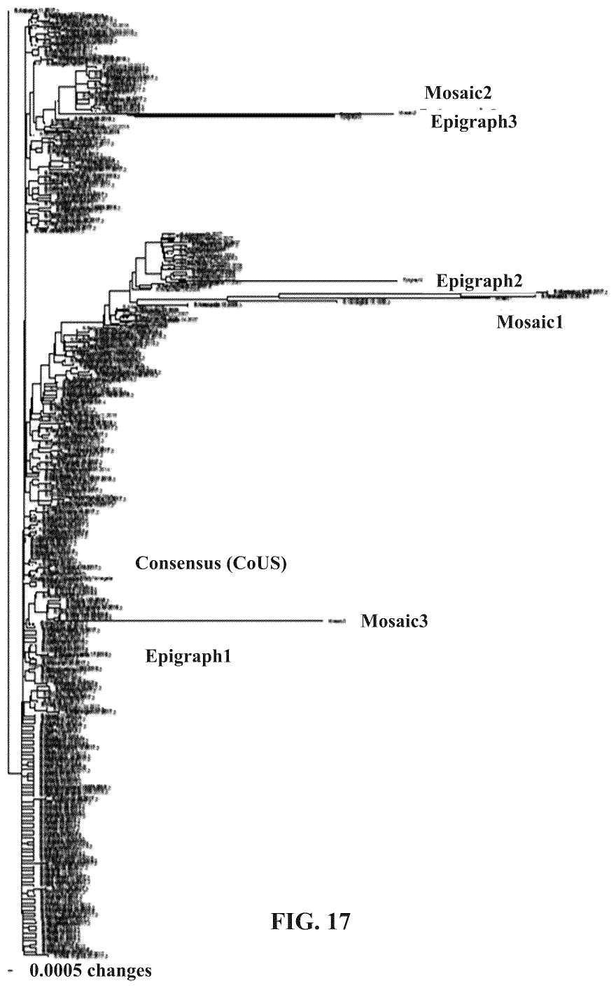

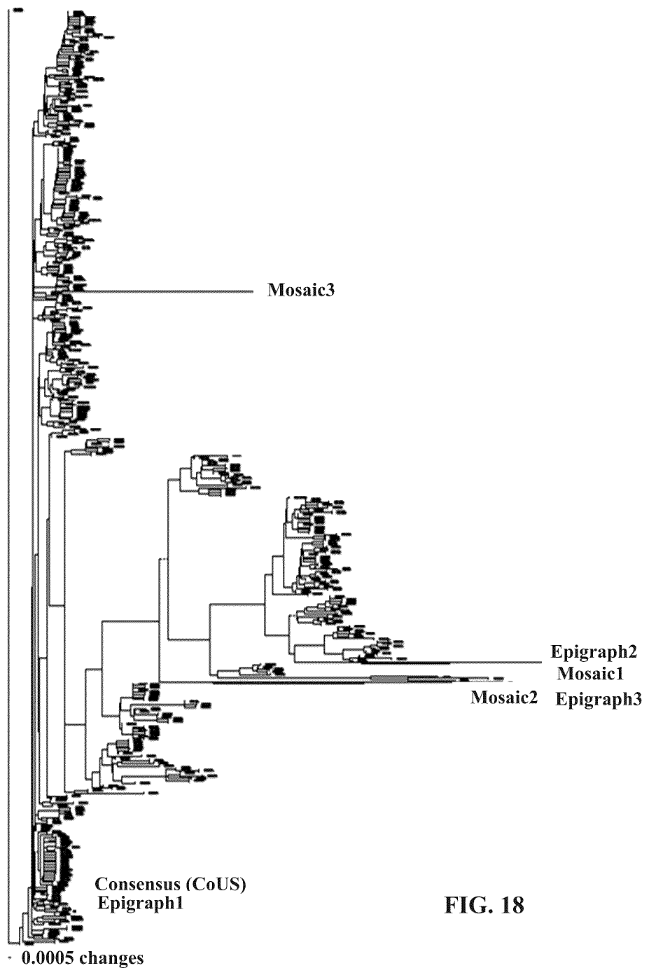

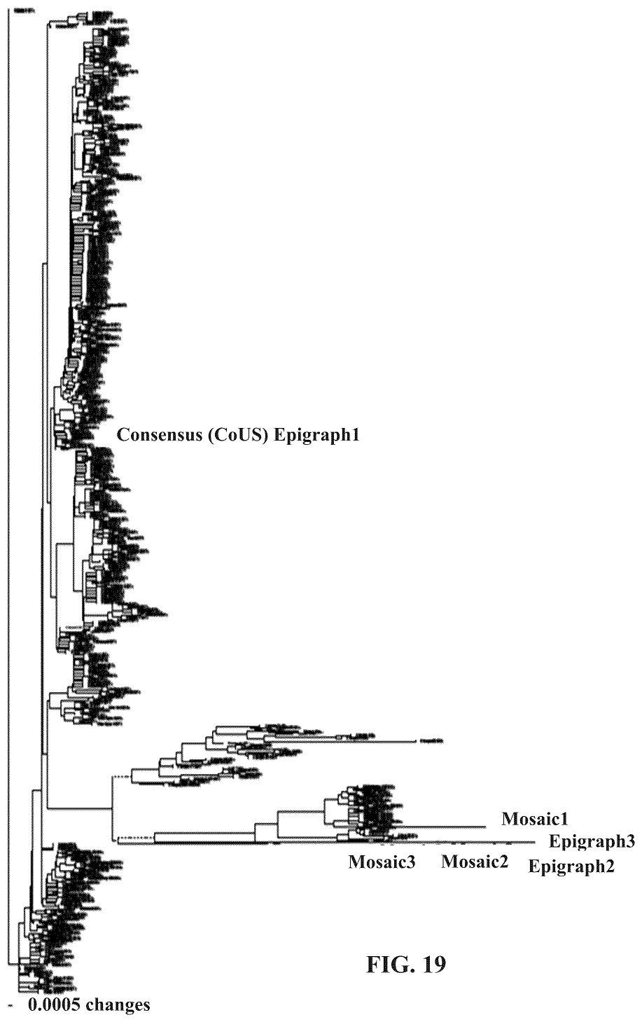

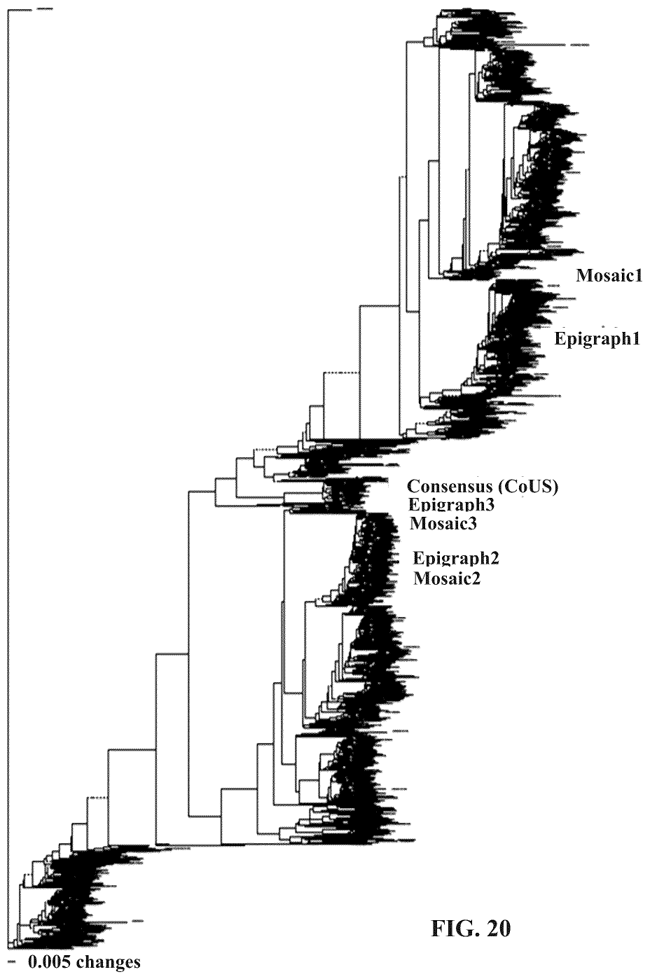

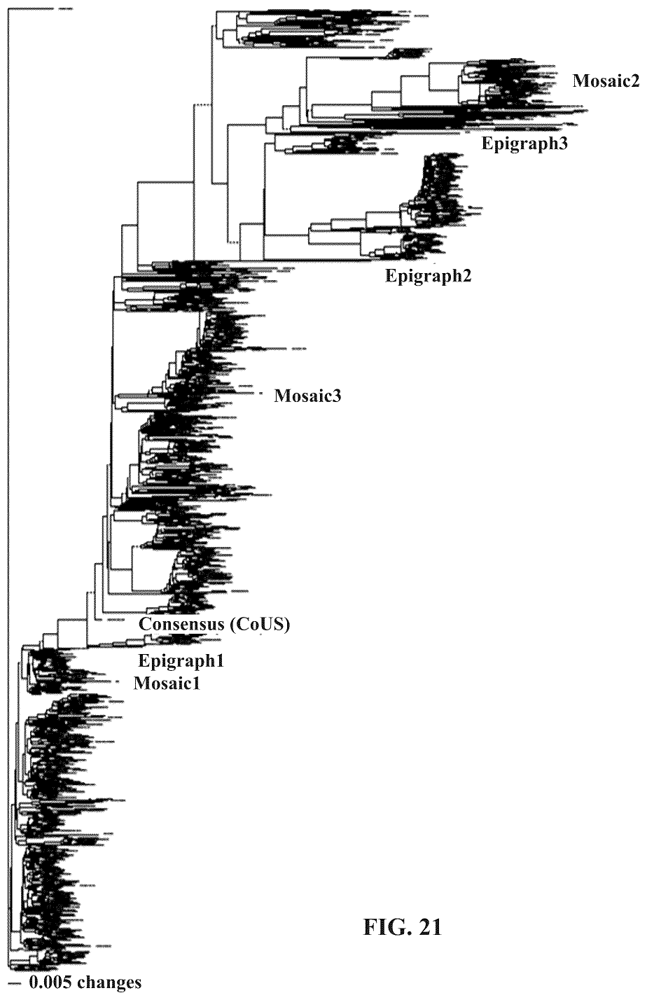

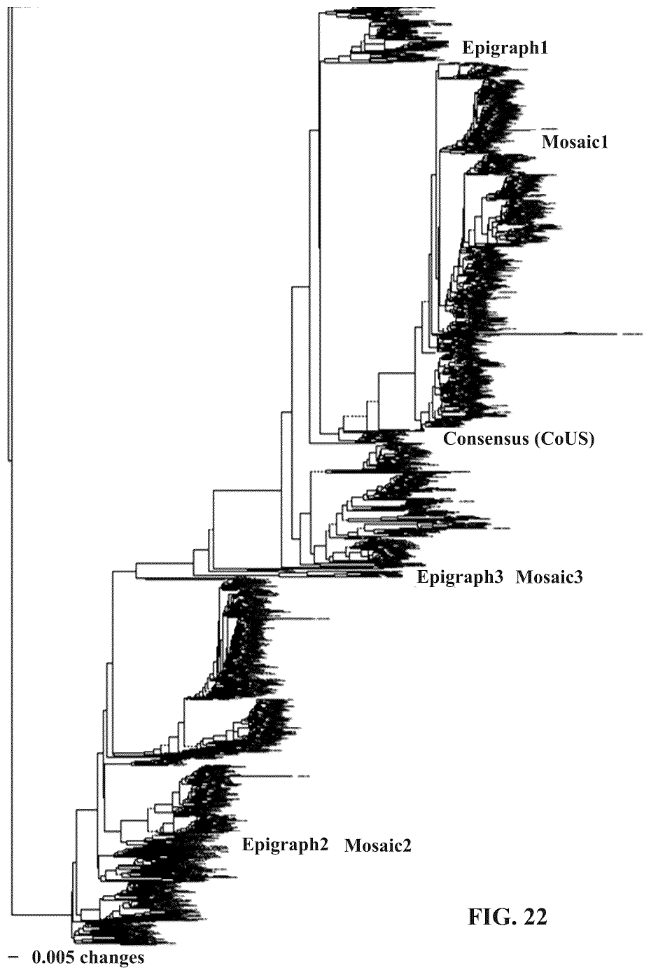

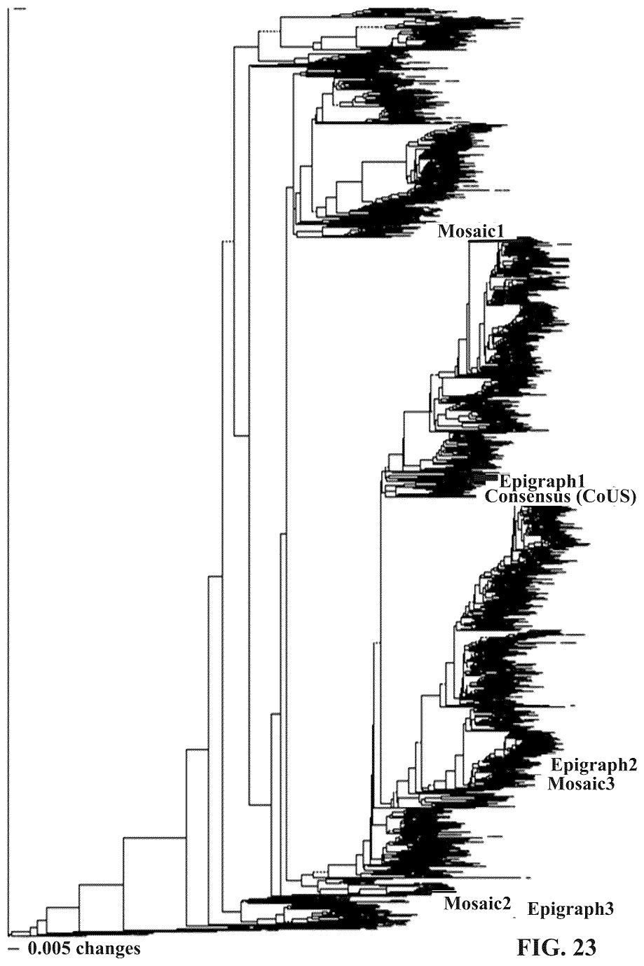

The genetic relationships between the vaccine genes described herein and wild type circulating genes are shown in FIGS. 9-23. Since the vaccine genes described herein were made using all unique HA sequences, they are not like any genes that would be made using other strategies such as Consensus, COBRA, Mosaics, and COT.

The unique universal vaccine genes described herein are for use against Human Influenza A Virus (H1, H2, H3, H5, N1 and N2 (SEQ ID NOs: 1-43)), Human Influenza B Virus (HA Yamagata-like, HA Victoria-like, NA Yamagata-like, NA Victoria-like (SEQ ID NOs: 44-74)) and Swine Influenza A Virus (H1, H3, N1 and N2 (SEQ ID NOs: 75-102)), and can be applied to the creation of a universal influenza vaccine that provides complete protection against all known influenza strains. Nucleic acid sequences encoding representative influenza vaccines are shown in SEQ ID NOs: 103-122.

### Example 2—Preliminary Results

IACUC. All animals were housed in the UNL Life Sciences Annex under the Association for Assessment and Accreditation of Laboratory Animal Care (AALAC) guidelines with animal use protocols approved by the corresponding the UNL IACUC protocol. All animal experiments were carried out according to the provisions of the Animal Welfare Act, PHS Animal Welfare Policy, the principles of the NIH Guide for the Care and Use of Laboratory Animals, and the policies and procedures of UNL.

Sex as a Biological Variable. Only female mice were used in the in vivo studies. However, all successful vaccine candidates were confirmed in studies using male mice.

Biohazard. All biohazard materials were subject to approval by the UNL Institutional Biosafety Committee (IBC). The UNL IBC was responsible for the safe use of infectious agents and recombinant DNA within UNL laboratories. This research did not use any reagents rated higher than BSL2.

Rigor and Transparency. In order to ensure statistical significance, groups of 5 mice and ferrets were used. In order to confirm reproducibility, all in vivo studies were repeated at least once. In addition, preliminary dose-dependent studies were performed that act as replicates within each experiment.

Production of Centralized COT H1, 2, 3, and 5 Genes. Comparison of select H1 HA proteins from 1933 to 2009 generated a phylogenetic tree with ˜21.0% sequence divergence across the branches. Due to high levels of genetic diversity, selecting a single wild type HA protein as a broadly cross-reactive vaccine was not thought to be feasible. Rather than select one wild type gene as a vaccine, a centralized gene was generated that mimics an ancestor of influenza infections during the past 78 years. The rationale for this was to produce an immunogen that is centrally located with respect to all other variants. Such a protein practically has lower sequence divergence with all of the variants as compared to any two randomly selected proteins. The centralized genes have been found to induce very effective levels of protection against divergent strains of influenza in BALB/c mice.

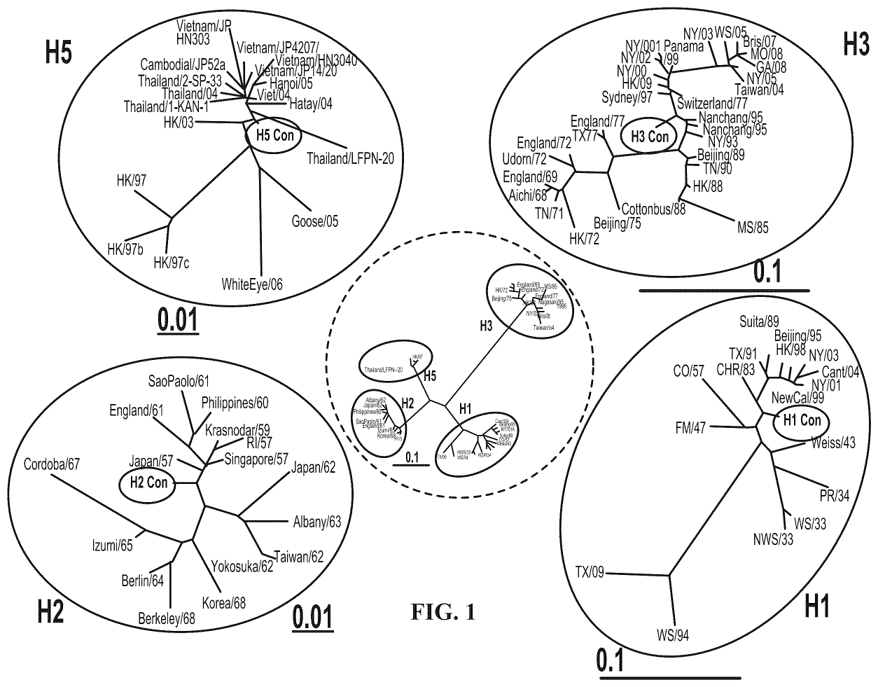

Phylogenetic Analyses of Centralized HA Genes. The relationship of the consensus COT genes to other wild type genes are shown in FIG. 1. All of the consensus genes localize to the central region of each of the respective trees. The average genetic distance of the H1, H2, H3 and H5 consensus genes to the wild type genes was 7.04%, 2.86%, 5.44% and 1.65% respectively. However, the maximum genetic distance between wild type H1, H2, H3 and H5 genes was 20.7%, 7.9%, 14.7% and 5.1% respectively. The centralized COT immunogens act to reduce genetic diversity to mismatched genes by being equidistant to all analyzed wild type genes (FIG. 1). The center tree shows the significant divergence between the influenza HA subtypes. The evolution of influenza allows for extreme level of plasticity in the HA genes, and the HA subtypes can be up to 60% divergent and still have identical functionality (FIG. 1).

In the case of mismatch, H1-COT induced better protection as compared to wild type immunogens.

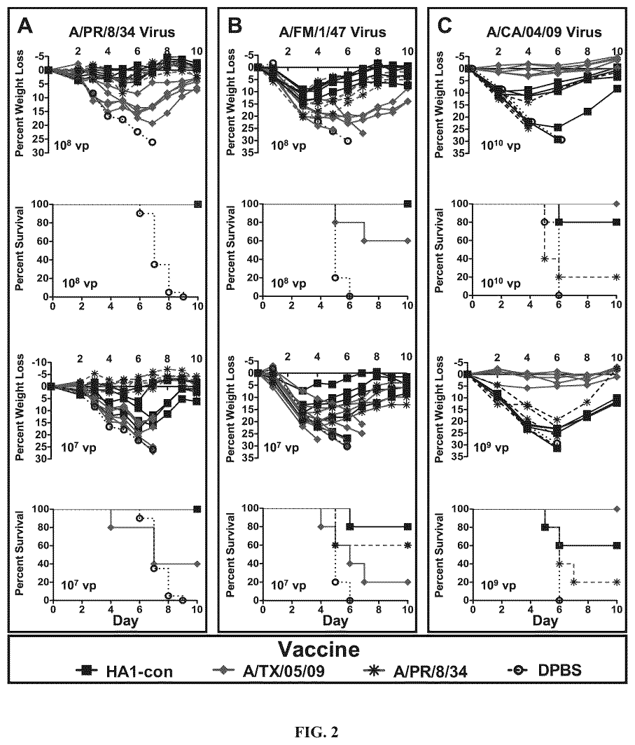

Protection Against Lethal A PR 8 34 Influenza. The homologous PR/8/34 vaccine was able to induce protective responses using the lowest dose of vaccine as compared to the mismatched vaccines, H1-COT and A/TX/05/09 (FIG. 2A). A/PR/8/34 immunized mice did not show any signs of disease or death with doses as low as 107 vp (FIG. 2A). However, mice vaccinated with H1-COT vaccine recovered and survived the challenge, whereas 60% of mice vaccinated with A/TX/05/09 at the same dose did not survive (FIG. 2A).

Protection Against Lethal A FM 1 47 Influenza. In this challenge study, all of the vaccines were mismatched to the challenge strain. The mice vaccinated with the H1-COT vaccine showed the least weight loss and disease as well as the highest level of survival against the lethal challenge (FIG. 2B). The centralized H1-COT vaccine was superior to the two wild type vaccine immunogens.

Protection Against Lethal A California 04 09. The matched CA/04/09 vaccine provided the best levels of protection against a lethal challenge with A/CA/04/09. However, when the vaccines were mismatched, the centralized H1-COT induced better protective immunity as compared to A/PR/8/34 (FIG. 2C). Importantly, 60% of H1-COT vaccinated mice survived, while only 20% of A/PR/8/34 vaccinated mice survived (FIG. 2C).

These data demonstrate that the centralized HA1-con immunogen is able to mediate cross-protection against a divergent influenza virus in three separate stringent challenge models, including 2009 H1N1.

This project will test the utility of centralized immunogens as foundation immunogens against other influenza strains.

Protection Against Divergent Lethal Challenge After Multivalent (H1+H2+H3+H5) (OT HA Vaccination. The ultimate proof of vaccine efficacy is a challenge study. Groups of mice were vaccinated with a multivalent vaccine consisting of four distinct Consensus COT HA subtypes, H1, H2, H3 and H5. It was successfully shown that multiple HA immunogens could be delivered simultaneously and the centralized immunogens provided 100% survival against 8 of 9 divergent lethal influenza viruses representing H1N1, H3N1, H3N2 and H5N1 influenza (Table 1). This challenge model was extremely stringent, and seven challenges consisted of 100 mouse lethal dose 50% (MLD50) and two challenges at 10 MLD50. In addition, there were no signs of disease in 7 out of 9 lethal challenges. This was very much in contrast to mice immunized with the traditional influenza vaccines, FluMist and FluZone. Mice immunized with these vaccines induced robust HI titers against 7 of 7 influenza viruses tested, however, they failed to provide any protection against disease in death when challenged with lethal influenza virus (Table 1). Interestingly, the universal multivalent vaccine also induced significantly higher T cell immunity and neutralizing antibody as compared to the traditional vaccines. The improved cellular and humoral immunity of the HA-COT vaccines can be selected for during the design and production of these universal vaccine immunogens.

TABLE 1
Summary of Supporting Data

| | Universal | | | | | |
|---|---|---|---|---|---|---|
| | Vaccine | | FluMist | | FluZone | |
| Hemmaglutination | 9/11 | (82%) | 7/7 | (100%) | 7/7 | (100%) |
| Inhibition Titers | | | | | | |
| Neutralization Titers | 6/6 | (100%) | 1/4 | (25%) | 1/4 | (25%) |
| Cellular Immunity | ++++ | | + | | + | |
| Linear Antibody Binding | ++ | | ++++ | | ++++ | |
| Linear Antibody Coverage | ++++ | | ++++ | | ++++ | |
| Protection Against Disease | 7/9 | (78%) | 0/5 | (0%) | 0/5 | (0%) |
| Survival | 8/9 | (89%) | 0/5 | (0%) | 0/5 | (0%) |

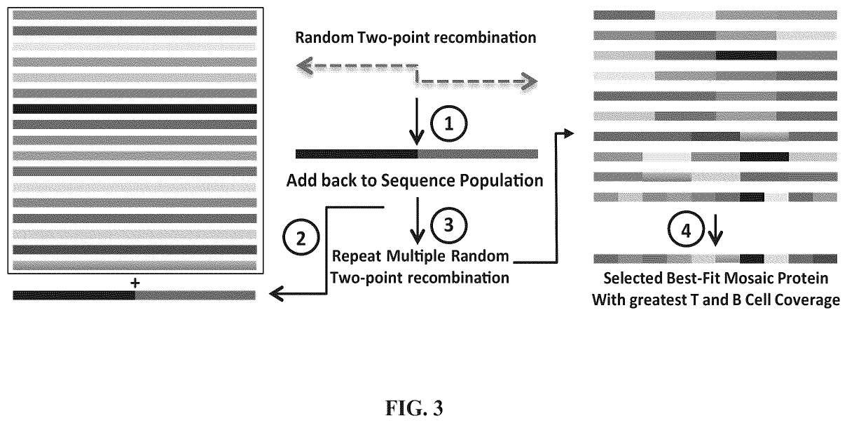

Construction of Human H1 and H3 COT, CoUS, Mosaic and Epigraph Candidate Vaccine Immunogens. Wild type vaccine immunogens are poorly immunogenic and provide strain-specific immunity (FIG. 4A). Center of tree (COT) strategies have been evaluated in which the vaccine immunogen is located to the central node of the phylogenetic tree and is, therefore, equidistant to all analyzed variants. This is advantageous since, the COT immunogen is most likely to be more closely related to a vaccine mismatch than any random wild type gene (FIG. 4B). The major disadvantage to this approach was that it gave equal weight to all major branches of the tree, some of which have only one or two unique strains. A second approach used only unique sequences to create the consensus immunogen, a consensus of unique sequences (CoUS). This had the advantage of giving equal weight to all know flu variants and was biased toward more contemporary strains. This strategy worked to localize the vaccine immunogen to give the greatest protection against the greatest number of unique sequences (FIG. 4C). A third universal vaccine strategy used in silico recombination with an algorithm that selected for recombinants that represent the most common repeated motifs. This Mosaic strategy is described in FIG. 3 and had the advantage of selecting for a universal vaccine immunogen that had been optimized for the greatest number of T and B cell epitopes (FIG. 4D). The final strategy utilized the same optimization of T and B cell epitopes as the Mosaic strategy, but, in this case, selected for a cocktail of vaccine immunogens that maximize vaccine coverage (FIG. 4E).

Parameters for creating the Human H1 candidate vaccine immunogens. In order to create the centralized human Influenza H1 HA genes, 6908 unique human HA protein sequences (duplicates excluded) were downloaded from the Influenza Research Database. The sequences were full-length and represent global coverage from the years 1918 to 2018. The consensus COT sequence was created as previously described (Weaver et al., 2011, PLOS One, 6: e18314). In order to create the consensus CoUS gene, only unique sequences were submitted to the Consensus Maker software program. In order to produce the Mosaic gene, the FASTA formatted sequences were submitted to the Mosaic Vaccine Designer at the Los Alamos National Laboratories and a Mosaic sequence was created using the following parameters: Cocktail Size: 1, Epitope Length: 9, Rare Threshold: 1, Run Time: 10 hrs, Population size: 200, Cycle Time: 10, Stall Time: 10, Internal Crossover Probability: 0.5. Mosaic Vaccine Sequence Coverage: 0.730394. The Epigraph sequences were created using the Epigraph vaccine design software with the following parameters: Algorithm: Unaligned sequence, Epitope Length: 9, Cocktail Size: 3. The consensus COT and CoUS, and optimized genes were introduced back into the original protein sequence file, realigned in ClustalW and the nexus output file was used to generate a neighbor-joining tree using PAUP 4.0a. The phylogenetic relationship of the centralized proteins to the natural proteins was identified.

Parameters for creating the Human H3 candidate vaccine immunogens. In order to create the consensus and optimized genes, 7313 unique human Influenza H3 HA protein sequences were downloaded from the Influenza Research Database. The sequences were full-length and represent global coverage from the years 1918 to 2018. The consensus COT was created as previously described (Weaver et al., 2011, PLOS One, 6: e18314). The unique H3 HA protein sequences were used to create the CoUS sequence using the Consensus Maker Software. The FASTA formatted sequences were submitted to the Mosaic Vaccine Designer at the Los Alamos National Laboratories and a mosaic sequence was created using the following parameters: Cocktail Size: 1, Epitope Length: 9, Rare Threshold: 1, Run Time: 10 hrs, Population size: 200, Cycle Time: 10, Stall Time: 10, Internal Crossover Probability: 0.5. Mosaic Vaccine Sequence Coverage: 0.827791. The Epigraph sequences were created using the Epigraph Software with the following parameters: Algorithm: Unaligned sequence, Epitope Length: 9, Cocktail Size: 3. The centralized genes were introduced back into the original protein sequence file, realigned in ClustalW and the nexus output file was used to generate a neighbor-joining tree using PAUP 4.0a. The phylogenetic relationship of the centralized proteins to the natural proteins was identified.

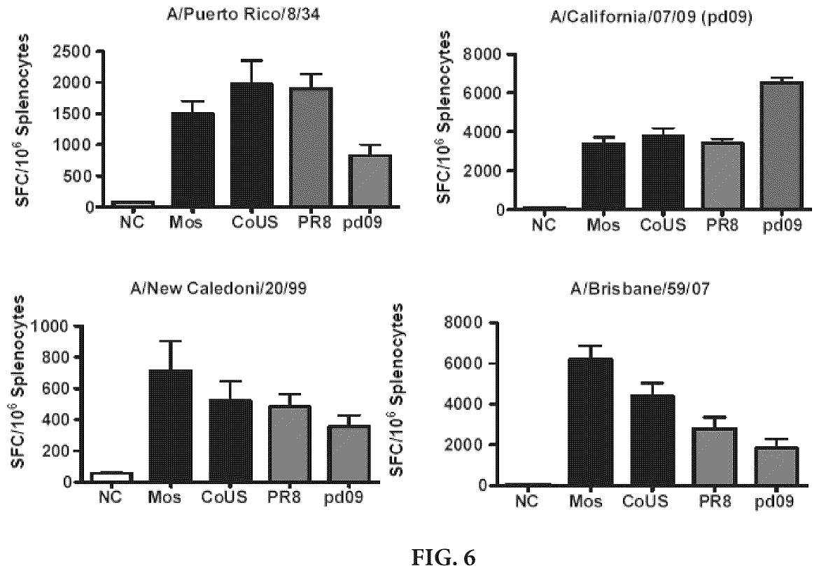

Improved Cellular Immune Responses In Novel HA Vaccine Immunogens. Mice were immunized with adenoviruses expressing novel human H1 Mosaic and CoUS genes and their ability to drive cellular immunity was compared against four divergent influenza viruses. When screened using an overlapping peptide library and ELISpot assay, it was found that in all cases of mismatch, the new Mosaic and CoUS genes induced greater levels of overall cellular immunity (FIG. 6). Interestingly, the Mosaic and CoUS genes induced T cell immunity against A/Puerto Rico/8/34 peptides equivalent to that of the vaccine expressing the homologous PR8 HA immunogen. In the case of the pd09 immune responses, the homologous HA vaccine induced the most robust immunity against the homologous peptides. However, in the case of vaccine mismatch, the Mosaic and CoUS HA vaccines induced equivalent or superior cellular immunity as compared to the wildtype PR8 and pd09 comparator vaccines (FIG. 6).

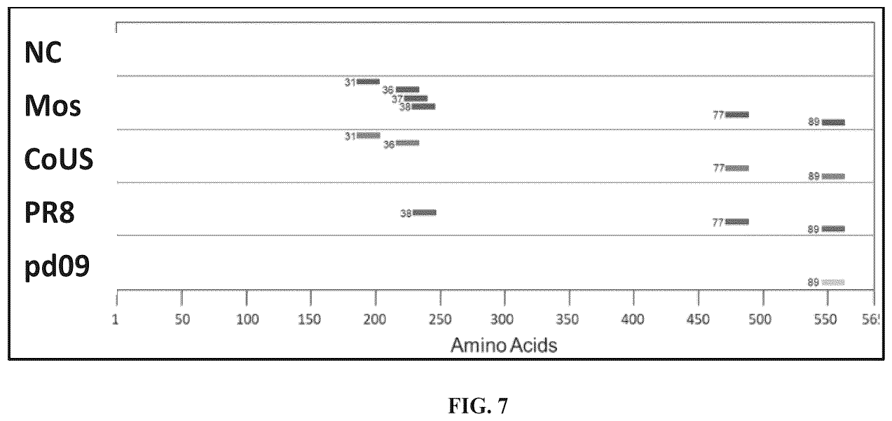

In addition, the T cell epitopes were mapped against the Brisbane peptide library. The Mosaic vaccine generated the greatest level of recognized peptides, followed by the CoUS vaccine immunogen with 6 and 4 peptides, respectively (FIG. 7). This was compared to the wild type responses to 3 and 1 peptides, respectively. The Mosaic, CoUS, PR8 and pd09 HA vaccines induced T cell responses against 5, 4, 3 and 1 epitopes, respectively (FIG. 7). This empirical data showed that the Mosaic vaccine strategy does indeed select for greatest T cell coverage, and likely B cell coverage, of all of the vaccine strategies.

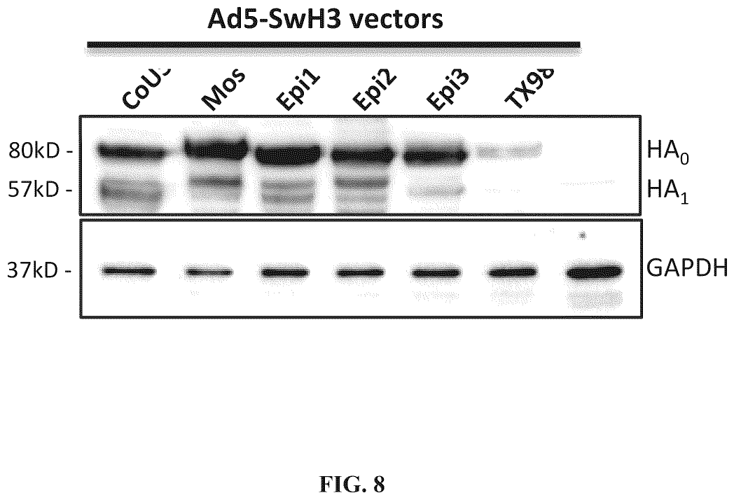

To determine if the unique immunogen design described herein would be better at inducing antibodies than wild type immunogens, cells were infected with equivalent infectious units of Ad-HA expressing viruses. The proteins were harvested and screened by Western blot. It was observed that all of the novel centralized and optimized vaccine immunogens were recognized more easily by serum from goats immunized with divergent A/swine/Indiana/0392/2011 virus as compared to the wild type TX98 HA protein. Since the novel vaccine immunogens were detected more readily as compared to the wild type TX/98 HA, this could indicate that these HA vaccines may be much better at presenting B cell epitopes and stimulating humoral immunity (FIG. 8).

Synthesis and cloning of wild type, COT, CoUS, Mosaic and Epigraph genes. The human HA Consensus COT, CoUS, Mosaic and Epigraph immunogens were computationally designed and analyzed. The genes were codon-optimized for humans since the target vaccines are human. The current 2018-19 Advisory Committee on Immunization Practices (ACIP) recommended wild type representative HA genes for both H1 and H3 be included. The wild type strain for use as the H1 comparator is A/Michigan/45/2015 (H1N1) pdm09-like. The wild type strain used as the H3 comparator was A/Singapore/INFIMH-16-0019/2016 (H3N2)-like virus. All of the codon-optimized HA genes were synthesized and cloned into pcDNA3.1 for T7 in vitro transcription (IVT). All of the vaccine genes are constructed synthetically by Genscript Corporation.

Production and quality control of mRNA and Lipid Nanoparticle (LNP) Vaccines. When comparing vaccine immunogens, it is essential that the quality of the vaccines are equivalent. Therefore, quality control of all mRNA-LNP vaccines were performed by analyzing the quality of mRNA by electrophoresis and densitometry analyses as well as quantitative protein expression by Western blot assays. All mRNA were produced using the HiScribe™ T7 ARCA mRNA Kit (with tailing).

Briefly, pcDNA3.1 plasmids containing the wild type and optimized vaccine immunogens were linearized using HindIII or XmaI, purified and used as template. RNA was transcribed from the template using the T7 RNA polymerase and incorporation of 5mCTP and pseudo-UTP for reduced innate immunity signaling. The RNA was capped using the ARCA capping enzyme and polyadenylated using the E. coli Poly (A) polymerase. The mRNA quality was confirmed by electrophoresis, quantitated by UV spectroscopy, aliquoted and stored at −80° C. The lipids DSPC, cholesterol (Sigma-Aldrich), DMAP-BLP, PEG-DMG2K and PEG-DSG2K (NOF America Corporation) were dissolved at a molar ratio of 50:10:39.5:0.5 in ethanol using microfluidic mixing. The mRNA was mixed with the lipid: ethanol solution at a 3:1 ratio in 50 mM Citrate (pH-4.0). The mRNA-LNP was dialyzed using 100K MWCO dialysis cartridges against PBS (pH-7.4) overnight and concentrated using Amicon Ultra Centrifuge filters. The final mRNA-LNP products were stored at 4° C. The mRNA-LNPs size and quality were confirmed by electron microscopy.

Dose titration of representative vaccine candidate in order to determine vaccine doses. Previous studies have shown that as little as 2 μg of mRNA-LNP can protect against a lethal Zika virus challenge. However, since we are using highly lethal influenza challenge models, we may need to immunize with higher doses of mRNA-LNP vaccine doses or use a prime boost strategy in order to achieve protective levels. We confirmed this protection by immunizing groups of 5 mice with a range of doses vaccine doses (2, 10 and 20 μg/mouse) and challenging at 5 weeks post-vaccination with homologous A/PR/8/34 virus. If complete protection was not observed at the highest dose, then a third group was primed and boosted with 20 μg/mouse. The mice were challenged 3 weeks post-boost with a lethal influenza challenge. If the prime/boost immunization failed to provide complete protection, then a prime/boost/boost vaccine strategy was explored in order to determine the optimal vaccine dose capable of providing complete protection.

Animals were anesthetized using ketamine/xylazine for all vaccinations and viral challenges. Intramuscular immunizations consisted of a 50 μl vaccine diluted in PBS and injected using a 27-gauge needle into both quadriceps in two 25 μl injections. Initially, all immunizations were done by the intramuscular route. All initial studies were done as single dose vaccine agents. Mice were challenged with 10-100 MLD50 of mouse-adapted influenza virus, and disease and death were monitored.

Immune correlates such as HI titers and T cell ELISpot assays. Immune correlates were analyzed against at least S-H1 and S-H3 influenza strains This data allowed us to extrapolate the true breadth and strength of the anti-influenza immunity induced by the various vaccines. Both cellular and humoral immunity were analyzed, as both play critical roles in protection and clearance. All candidate vaccine responses were compared to the commercial inactivated FluSure vaccine per manufacturer recommendations.

Hemagglutination Inhibition (HI) Assay. The primary determining factor for vaccine efficacy has always been the HI assay. Since all influenza viruses cannot be evaluated in mice, the HI assay allows the full evaluation of the cross-protective immune responses induced by the immunogens. Therefore, the HI assay was used to determine the induced cross-reactive antibodies.

Neutralization Assay. In order to determine the functional neutralization of the sera against homologous and heterologous sera, neutralization assays were performed.

Cellular Immunity (ELISpot). In order to determine the breadth and magnitude of the cellular immunity induced by the various vaccine immunogens, we epitope mapped and quantitated the absolute number of interferon secreting cells by IFN-γ ELISpot as previously described (REF 60) and shown in FIGS. 6 and 7.

Challenge study to determine most effective vaccine strategy. The most effective method of determining vaccine efficacy is the actual influenza challenge study. The H1 and H3 vaccines were tested separately in order to eliminate any potential interference between the vaccine immunogens. The vaccine groups consisted of single wild type, COT, CoUS or Mosaic HA immunogens. The epigraph immunogens were delivered as a cocktail. The HA-Epi vaccine consisted of the same final dose of mRNA-LNP as compared to the individual HA vaccines. Since the Epigraph vaccine was composed of a cocktail of 3 epigraph immunogens, each Epi mRNA was included in the vaccine at ⅓ of the final dose. For example, if the vaccine dose was 10 μg/mouse, the Epigraph vaccine consisted of 3.33 μg of each Epi mRNA-LNP vaccine. Groups of 5 control mice for each challenge virus were included. All of the vaccines were compared to a group that received the commercial quadrivalent FluZone at the mouse equivalent dose.

Determine if the vaccine candidates from the HI and H3 vaccine challenge studies can be combined into one universal vaccine against both subtypes. An ideal vaccine requires as few immunizations as possible and protects against all viral variants. Therefore, we determined if a single immunization comprised of the most effective approach for vaccinating against each influenza subtype could be combined to make a universal vaccine that induces immunity against H1N1 and H3N2 influenza virus. This assumes that one vaccine immunogen approach is superior for both H1 and H3 influenza subtypes. However, it is very likely that one approach is more effective than the other, so multivalent vaccines that are comprised of two different vaccine immunogens can be tested. For example, the epigraph approach may be more effective against H1N1 influenza, whereas the Mosaic approach may be more effective against H3N2 influenza. Therefore, the Ad-H1-Epi+Ad-H3-Mos multivalent vaccine were tested for efficacy against all six influenza viruses. Groups of 5 control mice for each challenge virus were included.

Immunization in the presence of prior immunity due to previous vaccination. These experiments simulate an immune system that has been primed by a seasonal inactivated vaccine and not a natural infection. These conditions more accurately resemble the immune systems of vaccinated children. Groups of five mice were immunized with a seasonal inactivated FluZone vaccine. The mice were re-immunized six months later with either wild type or the universal multivalent optimized vaccine. The mice were challenged with a panel of influenza viruses to determine the degree of cross-protective immunity. All mice studies were repeated.

Immunization in the presence of prior immunity due to natural infection. In order to simulate immunity derived from a natural infection, mice were infected with a low dose of wild type influenza virus. These conditions more accurately resemble the immune systems of adults. The strain-specific wild type and universal vaccines were tested for their ability to induce cross-protective immunity in the context of this immune repertoire in the same manner as previously described for animals with prior immunity due to vaccination. All mice studies were repeated.

Influenza animal model. The ferret model closely mimics influenza infection in humans. Ferrets have similar clinical symptoms and are equally sensitive to influenza infection. The ferret model is considered the best animal model to evaluate influenza pathogenicity, virulence, infectivity, and vaccine efficacy (Herlocher et al., 2001, J. Infect. Dis., 184:542-6; Maassab et al., 1982, J. Infect. Dis., 146:780-90; Sweet & Smith, 1980, Microbiol. Rev., 44:303-30). Also, since the preliminary evaluation of cross-protective immune responses is done using BALB/c mice, it is important to determine the immune responses in an outbred genetic background. Therefore, the ferret model was used for a final evaluation of the most efficacious vaccines. All ferret studies were repeated.

Ferrets were used in the final analysis of the most promising broadly protective vaccines. Groups of five ferrets were immunized with the wild type, universal, and FluZone vaccines. Sera and nasal washes were obtained three weeks post-immunization. These samples were assayed against a large panel of influenza viruses using HI and micro-neutralization assays for a broader analysis of cross-protective immune responses. The immunized ferrets were challenged with at least four representative viruses from each subtype. The ferrets were monitored daily for temperature and dehydration. Fever in ferrets rarely approaches dangerous levels and there is some indication that reducing fever actually increases viremia. If the animals were dehydrated, they were treated daily by subcutaneous injection of 10 ml of sterile saline. Moribund animals were sacrificed. Disease as measured by weight loss, clinical score and nasal virus shedding was used to evaluate the protective responses of each vaccine.

### Example 3—Representative Sequence Comparisons

The sequences of select vaccine polypeptides described herein were compared to one another as shown in Table 1.

TABLE 1
Comparison of Select Influenza H1 HA Protein Sequences

| | A/Brisbane/ | A/NC/ | A/PR/ | A/FM/ | | A/CA/ | A/TX/ | A/Nanachang/ | HuH1 |
|---|---|---|---|---|---|---|---|---|---|
| Strain | 59/07 | 20/99 | 8/34 | 1/47 | A/WS/33 | 07/09 | 05/09 | 1/99 | Mosaic |
| Names | Identity Scores (%) | | | | | | | | |
| A/Brisbane/ | 100.0 | 97.5 | 86.5 | 89.6 | 86.0 | 79.7 | 79.3 | | 97.2 |
| 59/07 | | | | | | | | | |
| A/NC/20/99 | 98.4 | 100.0 | 88.1 | 91.3 | 87.8 | 80.0 | 79.7 | | 95.1 |
| A/PR/8/34 | 92.0 | 93.3 | 100.0 | 90.8 | 92.2 | 81.6 | 81.3 | | 84.8 |
| A/FM/1/47 | 93.8 | 95.2 | 94.5 | 100.0 | 90.8 | 81.1 | 80.7 | | 87.2 |
| A/WS/33 | 91.7 | 93.1 | 95.1 | 94.7 | 100.0 | 82.2 | 81.8 | | 84.3 |
| A/CA/07/09 | 88.7 | 89.6 | 89.6 | 89.4 | 90.5 | 100.0 | 99.6 | | 77.3 |
| A/TX/05/09 | 88.5 | 89.4 | 89.4 | 89.2 | 90.3 | 99.8 | 100.0 | | 76.9 |
| A/Nanachang/ | | | | | | | | 100 | |
| 1/99 | | | | | | | | | |
| HuH1 | 98.4 | 96.8 | 90.9 | 92.3 | 90.6 | 87.2 | 87.1 | | 100 |
| Mosaic | | | | | | | | | |

Similarity Scores (%)

### Example 4—Western Blot for the Detection of Vaccine Immunogens

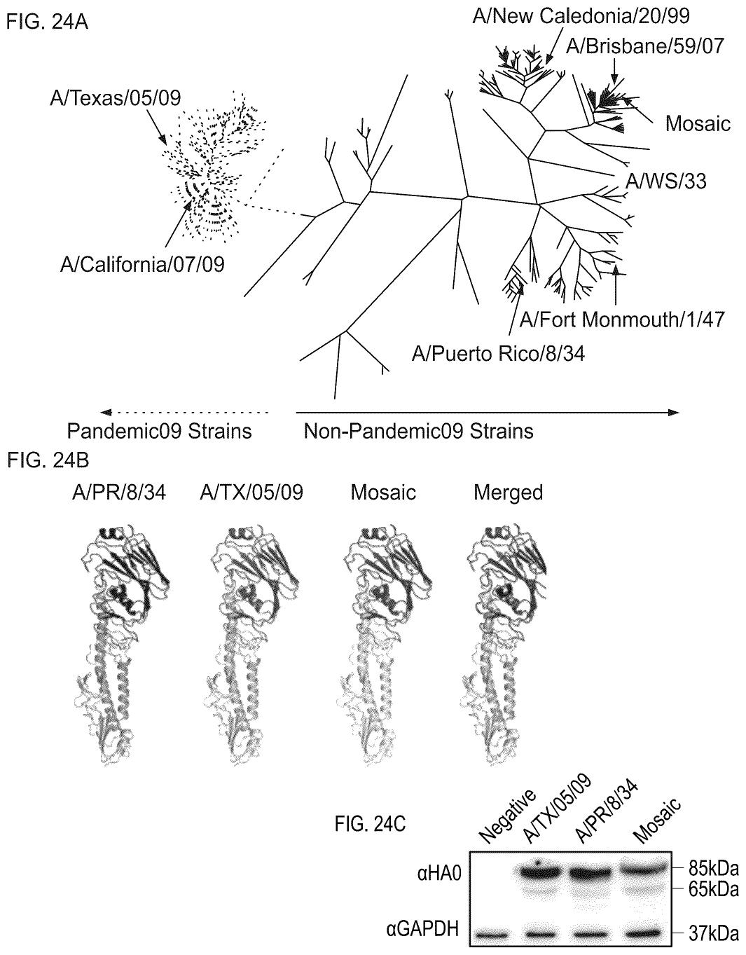

293 cells were infected with 500 virus particles per cell and harvested 48 hours later. The Western blot was probed with anti-HA0 antibody (NR-3148) from the A/PR/8/34 strain and anti-HA1 antibody as the primary antibodies. Both the HA0 (85 kDa) and HA1 (65 kDa) bands were observed for all three recombinant adenoviruses and were expressed at similar levels (FIG. 24C).

Female Balb/c mice were immunized with 10e10 virus particles of recombinant Ad5-H1-Mosaic-HA, Ad5-A/PR/8/34-HA, Ad5-A/TX/05/09-HA (pdm09), 150 ng of Fluzone HA, or PBS and sera was harvested 3 weeks later (n=8). Sera was treated with RDE and measured for antibodies using HI titers starting at a 1:10 dilution. Influenza strains A/CA/07/09 (FIG. 25A), A/NC/20/99 (FIG. 25B), A/PR/8/34 (FIG. 25C), A/WS/33 (FIG. 25D), and A/FM/1/47 (FIG. 25E) were measured for antibodies by hemagglutination inhibition assay. Results are shown as the reciprocal of the highest dilution that prevented agglutination, and negative results are shown as half the limit of detection.

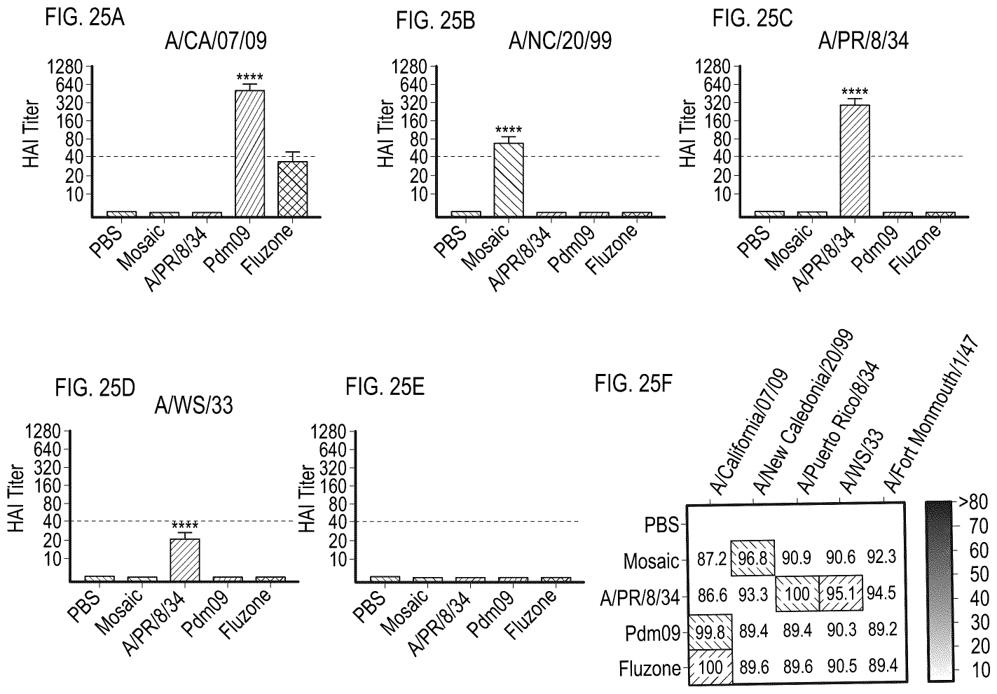

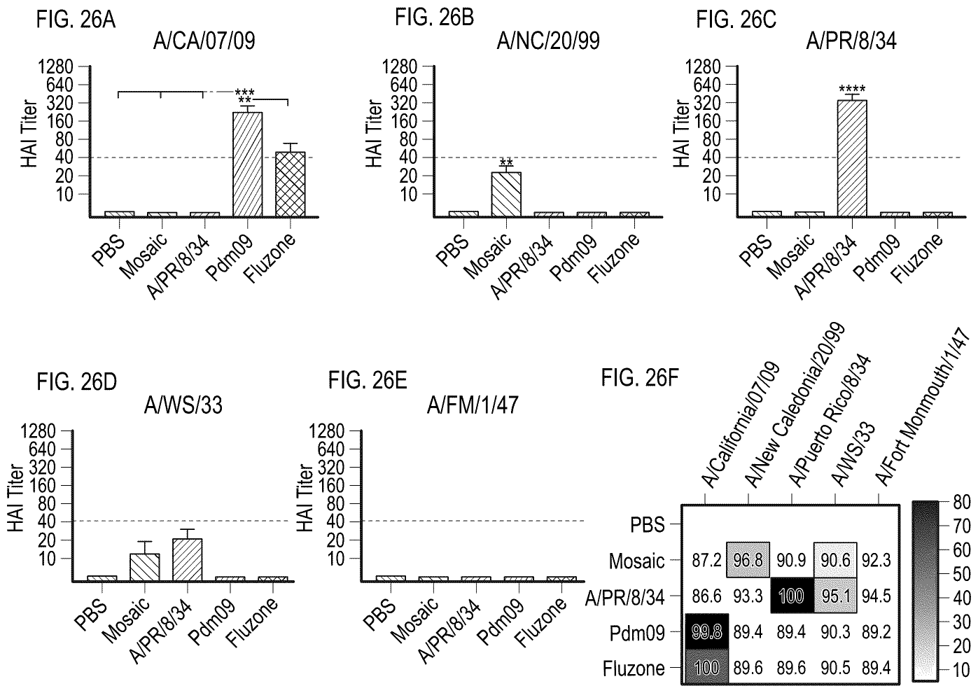

Female Balb/c mice were immunized with 10e10 virus particles, 150 ng of Fluzone HA, or PBS and boosted with the same dose 3 weeks later (n=5). Sera was harvested 10 days post-boost and treated with RDE to be measured for antibodies using HI titers starting at a 1:10 dilution. Influenza strains A/CA/07/09 (FIG. 26A), A/NC/20/99 (FIG. 26B), A/PR/8/34 (FIG. 26C), A/WS/33 (FIG. 26D), and A/FM/1/47 (FIG. 26E) were measured for antibodies by hemagglutination inhibition assay. Results are shown as the reciprocal of the highest dilution that prevented agglutination, and negative results are shown as half the limit of detection.

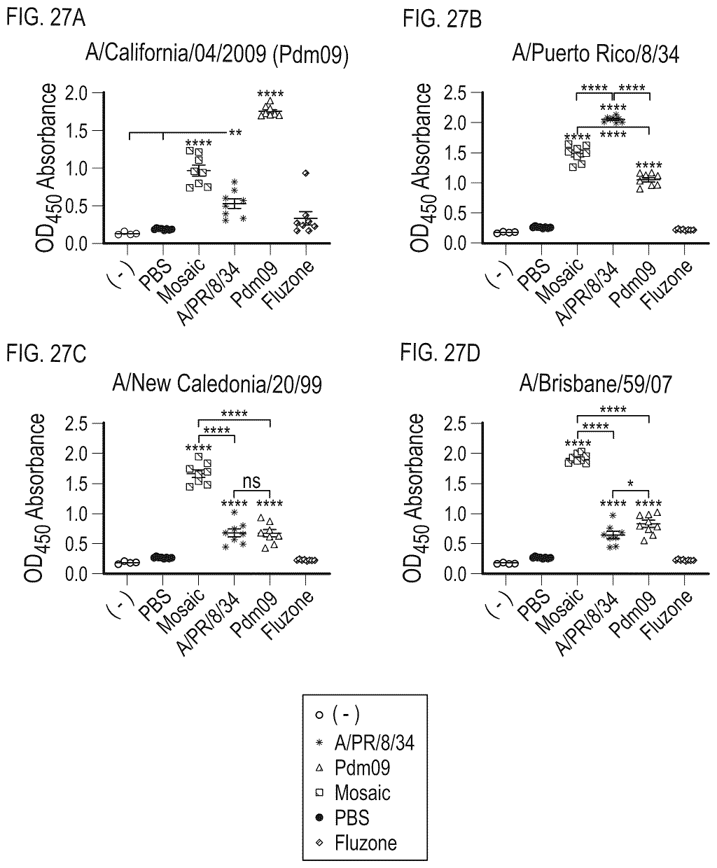

Female Balb/c mice were immunized with 10e10 virus particles of recombinant Ad5-H1-Mosaic-HA, Ad5-A/PR/8/34-HA, Ad5-A/TX/05/09-HA (pdm09), 150 ng of Fluzone HA, or PBS, and sera was harvested 3 weeks later (n=8). Sera was diluted 1:100 and measured for antibodies using ELISA assays. Plates were coated with 200 ng per well of recombinant HA protein from influenza strains A/CA/04/09 (FIG. 27A), A/PR/8/34 (FIG. 27B), A/NC/20/99 (FIG. 27C), and A/Brisbane/59/07 (FIG. 27D) and the absorbance at OD450 measured.

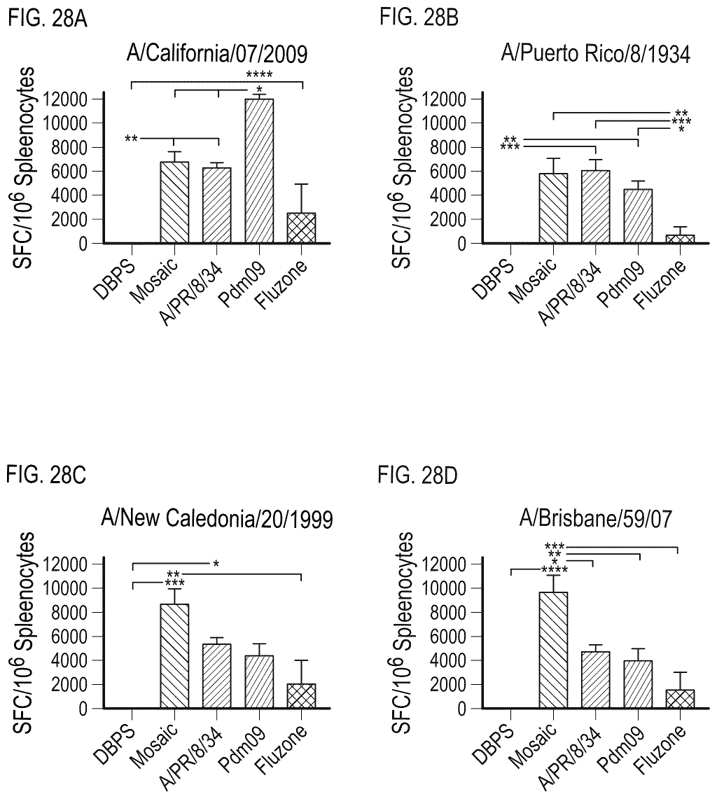

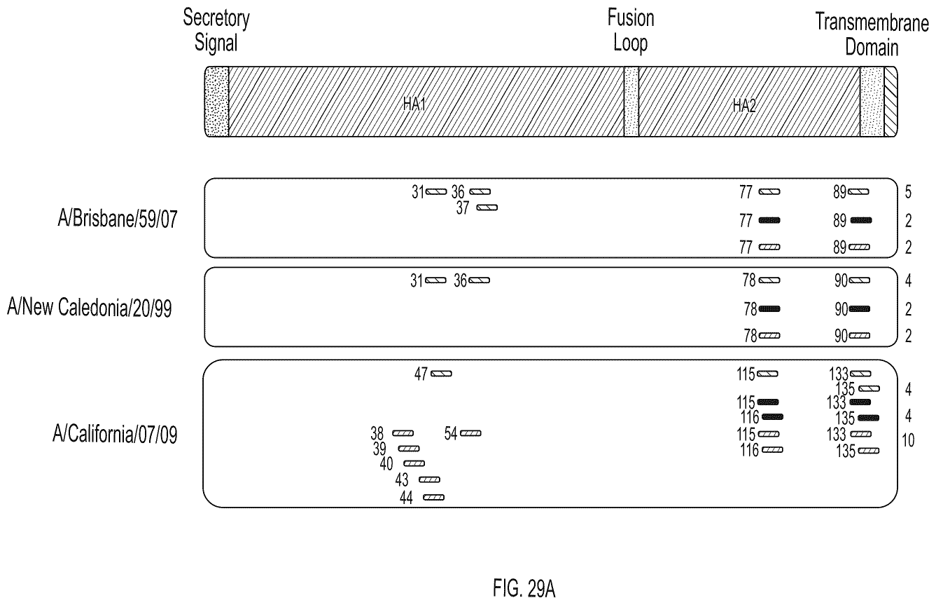

Female Balb/c mice were immunized with 10e10 virus particles of recombinant Ad5-H1-Mosaic-HA, Ad5-A/PR/8/34-HA, Ad5-A/TX/05/09-HA (pdm09), 150 ng of Fluzone HA, or PBS, and splenocytes were harvested 3 weeks later (n=8). Total T cell responses were measured by IFNgamma ELISpot assay using peptide arrays spanning the entire HA gene of A/California/07/09 (FIG. 28A), A/Puerto Rico/8/34 (FIG. 28B), A/New Caledonia/20/99 (FIG. 28C), or A/Brisbane/59/07 (FIG. 28D) strains and expressed as the mean number of spot forming cells (SFC) per million splenocytes.

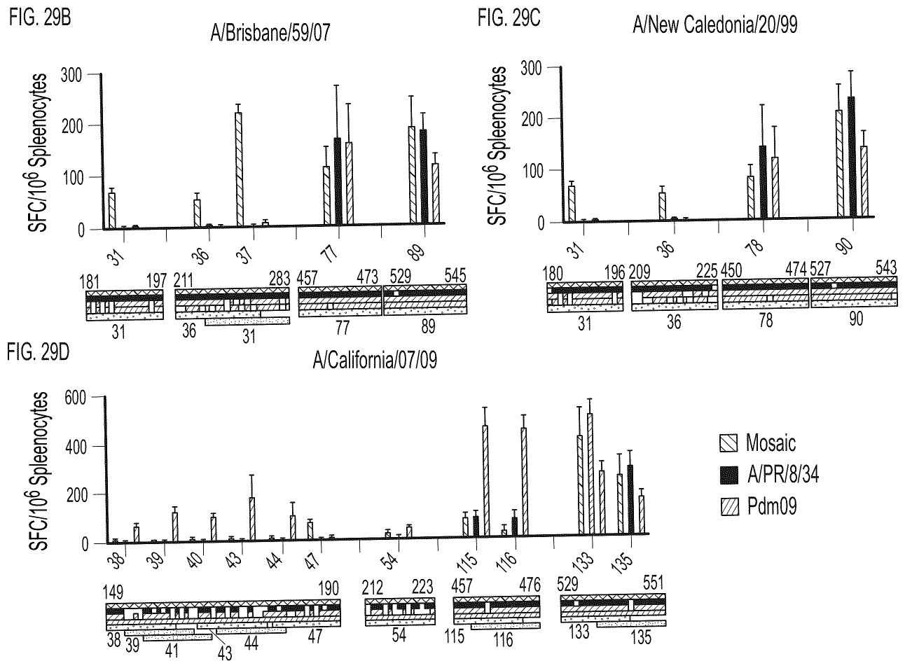

Female Balb/c mice were immunized with 10e10 virus particles of recombinant Ad5-H1-Mosaic-HA, Ad5-A/PR/8/34-HA, Ad5-A/TX/05/09-HA (pdm09), 150 ng of Fluzone HA, or PBS and splenocytes were harvested 3 weeks later (n=8). T cell epitopes were mapped using overlapping peptide libraries spanning the entire HA gene. Responses greater than 50 spot forming cells (SFC) per million splenocytes were considered positive and are shown in relation to the HA gene location (FIG. 29A). Positive peptides for A/Brisbane/59/07 (FIG. 29B), A/New Caledonia/20/99 (FIG. 29C), and A/California/07/09 (FIG. 29D) are quantified. An alignment of the peptide strain (grey), mosaic (green), A/PR/8/34 (dark blue), and A/TX/05/09 pdm09 (light blue) HA sequences are shown directly under the positive peptide indicating the possible epitope locations.

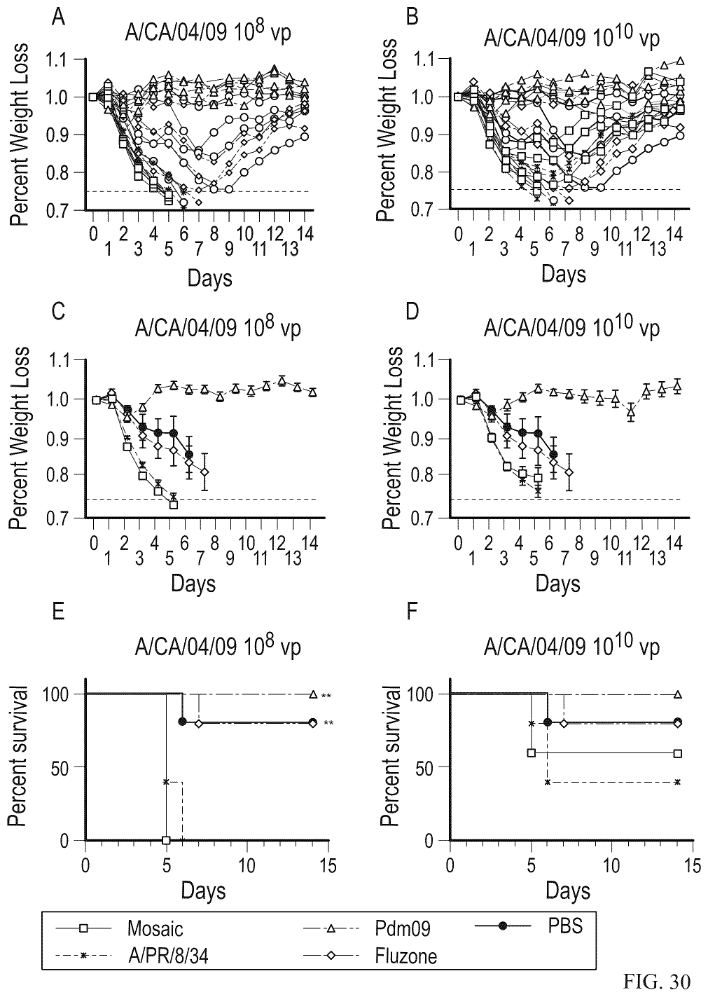

Female Balb/c mice were immunized with either 10e8 or 10e10 virus particles of recombinant Ad5-H1-Mosaic-HA, Ad5-A/PR/8/34-HA, Ad5-A/TX/05/09-HA (pdm09), 150 ng of Fluzone HA, or PBS and challenged with 100MLD50 3 weeks later (n=5). Mice were monitored 14 days post challenge and humanely euthanized if 25% weight loss was recorded. Individual mice were monitored for weight loss (FIG. 30A, 30B), average weight loss (FIG. 30C, 30D), and percent survival (FIG. 30E, 30F).

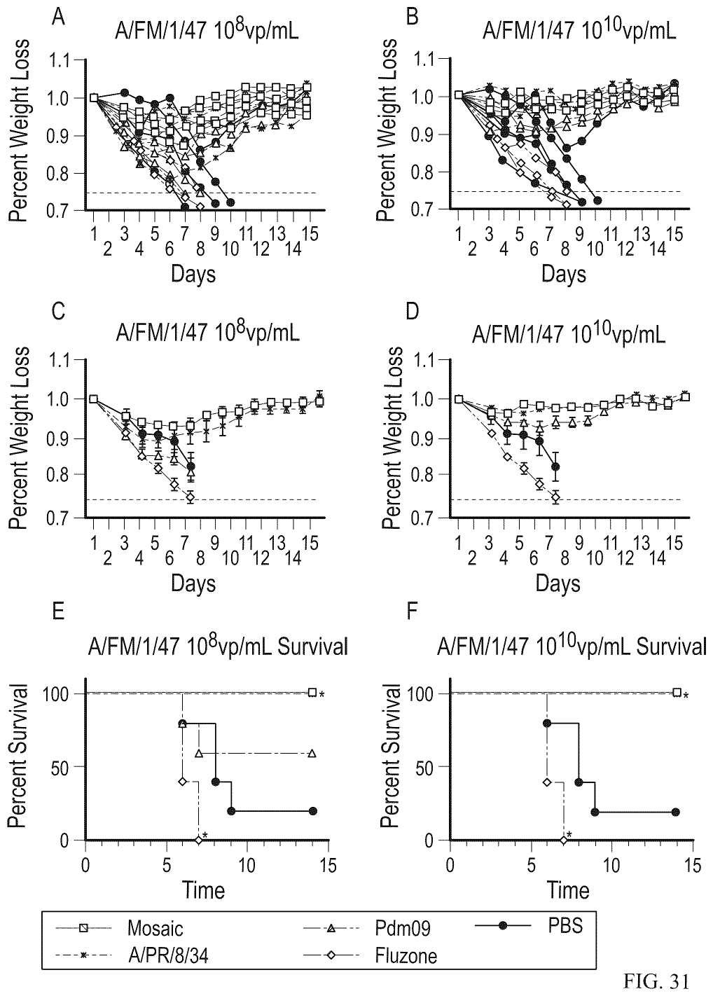

Female Balb/c mice were immunized with either 10e8 or 10e10 virus particles of recombinant Ad5-H1-Mosaic-HA, Ad5-A/PR/8/34-HA, Ad5-A/TX/05/09-HA (pdm09), 150 ng of Fluzone HA, or PBS and challenged with 100 mouse lethal dose (MLD) 50 3 weeks later (n=5). Mice were monitored 14 days post challenge and humanely euthanized if 25% weight loss was recorded. Weight loss for individual mice (FIG. 31A, 31B), average weight loss (FIG. 31C, 31D), and percent survival (FIG. 31E, 31F) were determined.

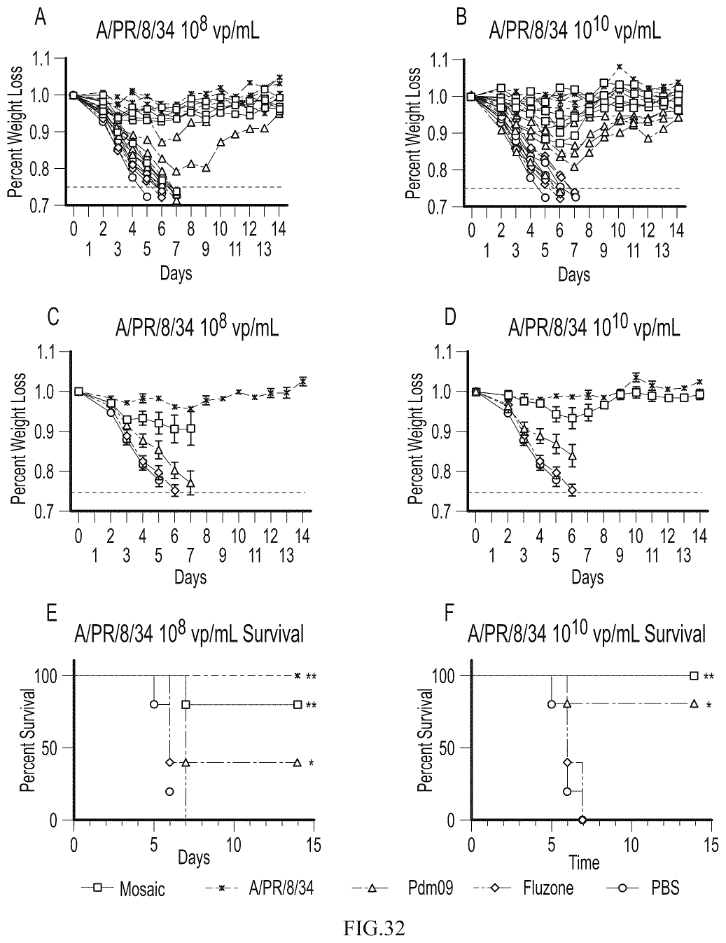

Female Balb/c mice were immunized with either 10e8 or 10e10 virus particles of recombinant Ad5-H1-Mosaic-HA, Ad5-A/PR/8/34-HA, Ad5-A/TX/05/09-HA (pdm09), 150 ng of Fluzone HA, or PBS and challenged with 100MLD50 3 weeks later (n=5). Mice were monitored 14 days post challenge and humanely euthanized if 25% weight loss was recorded. Weight loss for individual mice (FIG. 32A, 32B), average weight loss (FIG. 32C, 32D), and percent survival (FIG. 32E, 32F) were determined.

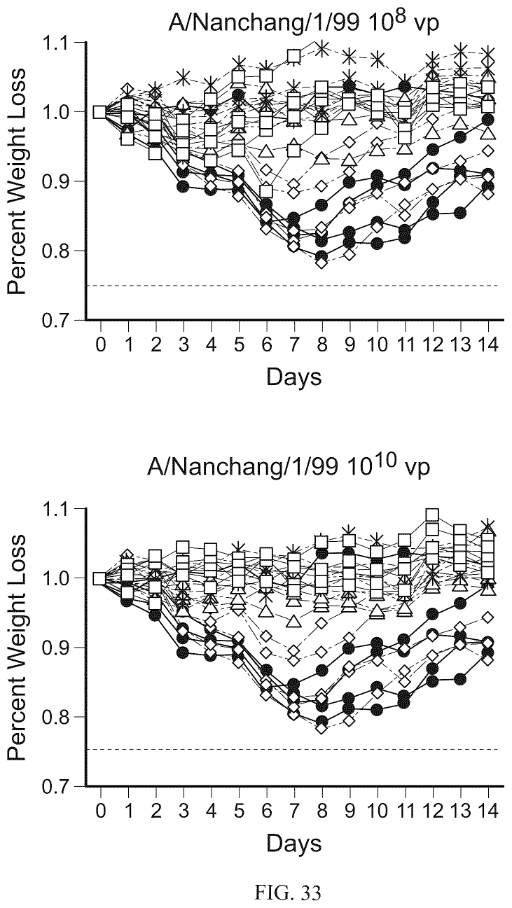

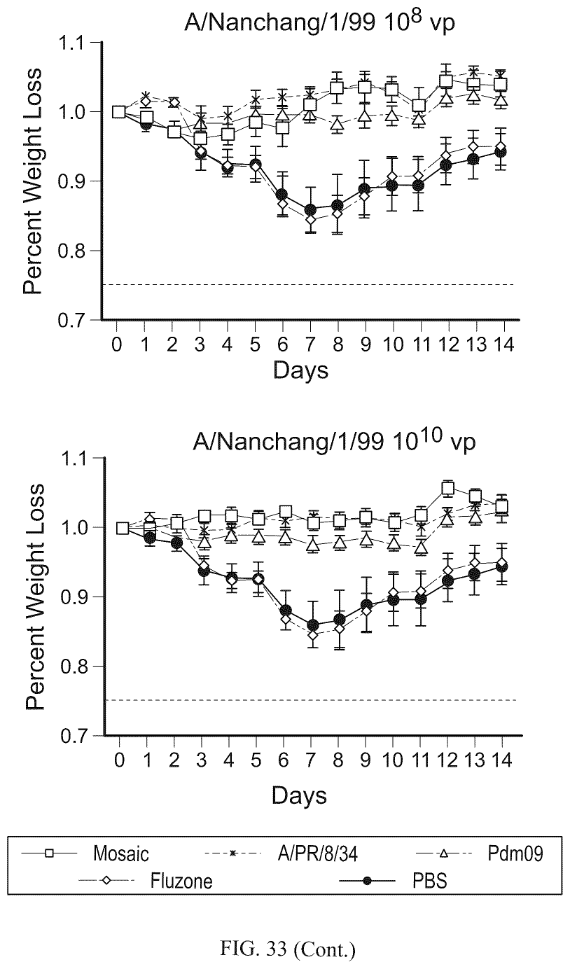

Female Balb/c mice were immunized with either 10e8 or 10e10 virus particles of recombinant Ad5-H1-Mosaic-HA, Ad5-A/PR/8/34-HA, Ad5-A/TX/05/09-HA (pdm09), 150 ng of Fluzone HA, or PBS and challenged with 100MLD50 3 weeks later (n=5). Mice were monitored 14 days post-challenge and humanely euthanized if 25% weight loss was recorded. Weight loss for individual mice (FIG. 33A, 33B), average weight loss (FIG. 33C, 33D), and percent survival (FIG. 33E, 33F) were determined.

### Example 5—Phylogenetic Tree of Swine Influenza and Universal Vaccine Hemagglutinins

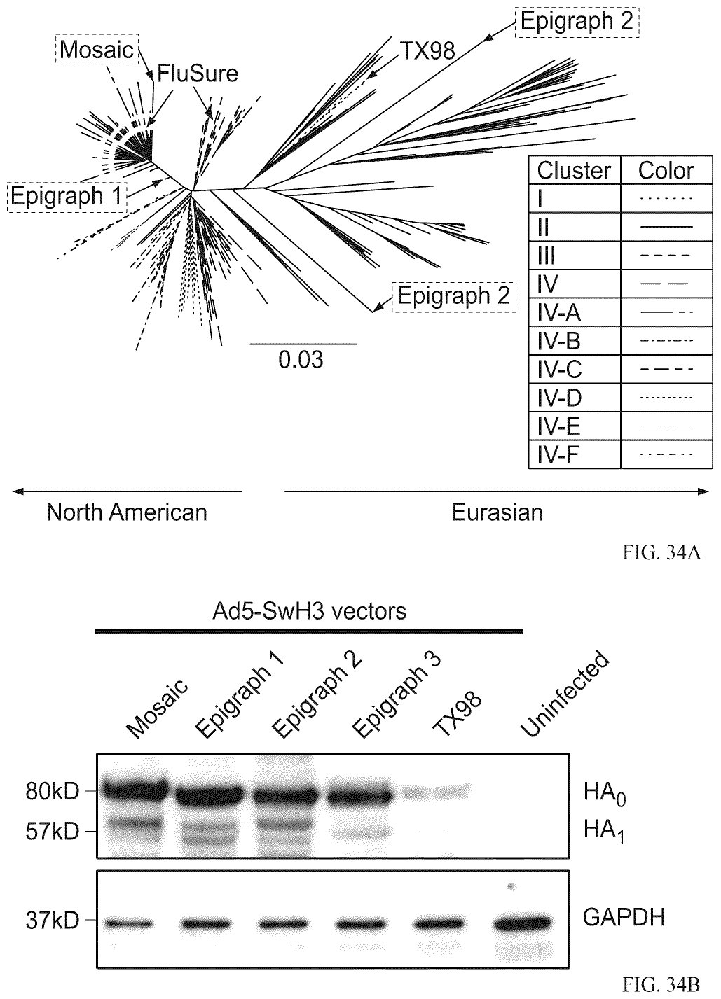

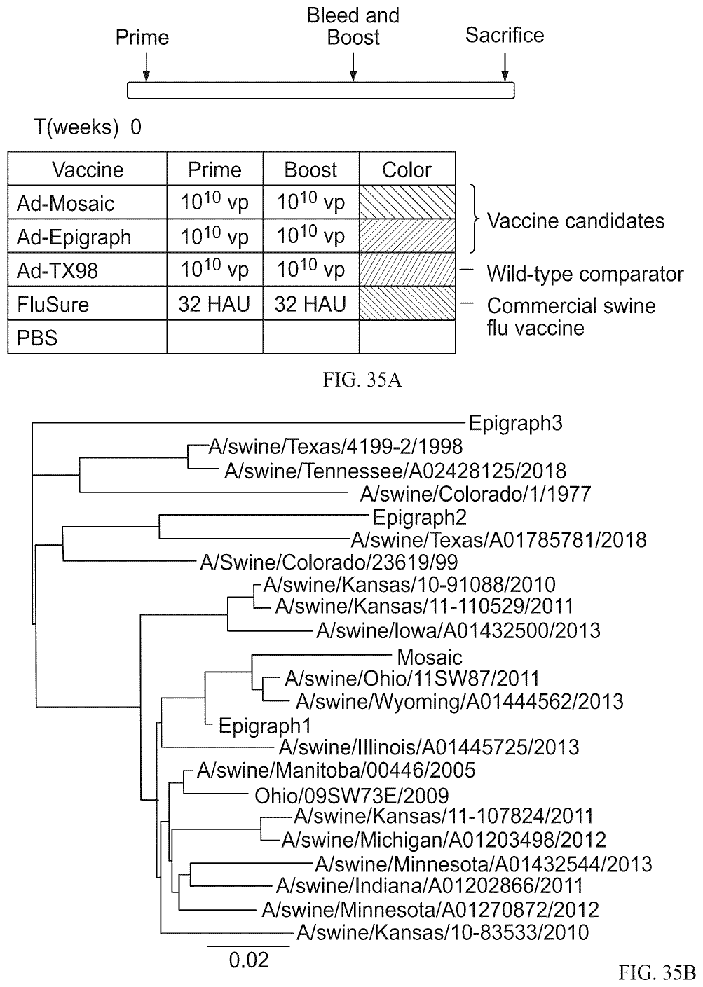

All 1,561 unique swine H3 Influenza virus HA proteins (excluding laboratory strains and duplicate strains) were downloaded from the Influenza Research Database. The Mosaic Vaccine Design and Epigraph programs available through the HIV database tools suite were used to create the Mosaic and Epigraph candidate vaccine proteins, respectively. The Mosaic and three Epigraph immunogens were then aligned with the original sequence population using ClustalW and a Jukes-Cantor neighbor joining tree was constructed (FIG. 34A). These universal immunogens were synthesized and cloned into a replication-defective Adenovirus 5 vector. 293 cells were infected with equivalent infectious units, using a wild type comparator HA gene (A/swine/Texas/4199-2/1998 [TX98]) as a control, and Hemagglutinin protein expression was detected by probing the Western blots with polyclonal Goat anti A/swine/Indiana/0392/2011 serum (FIG. 34B).

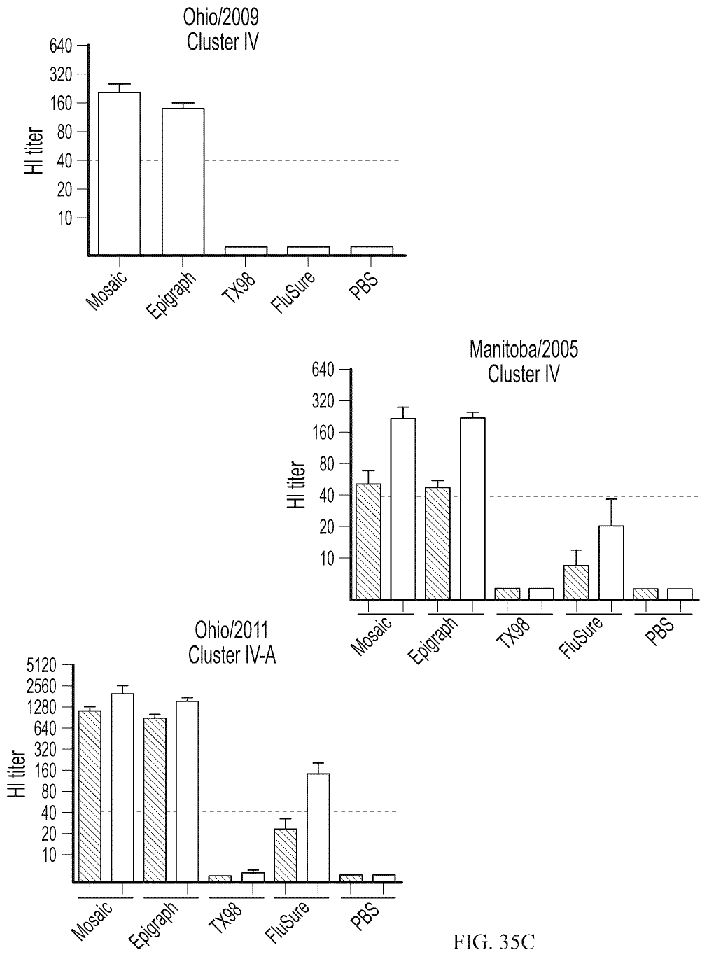

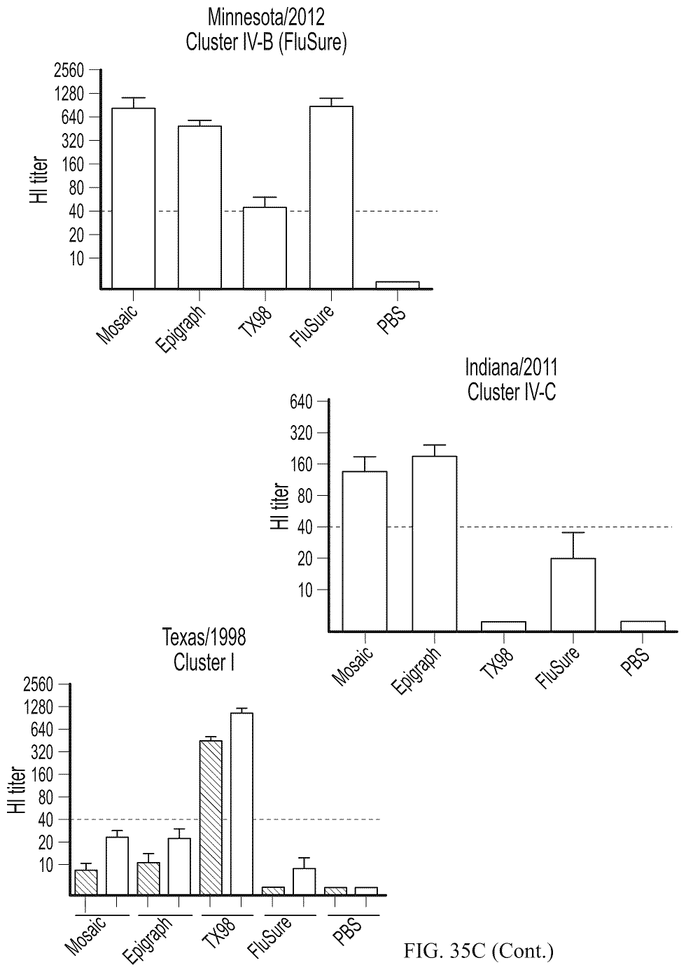

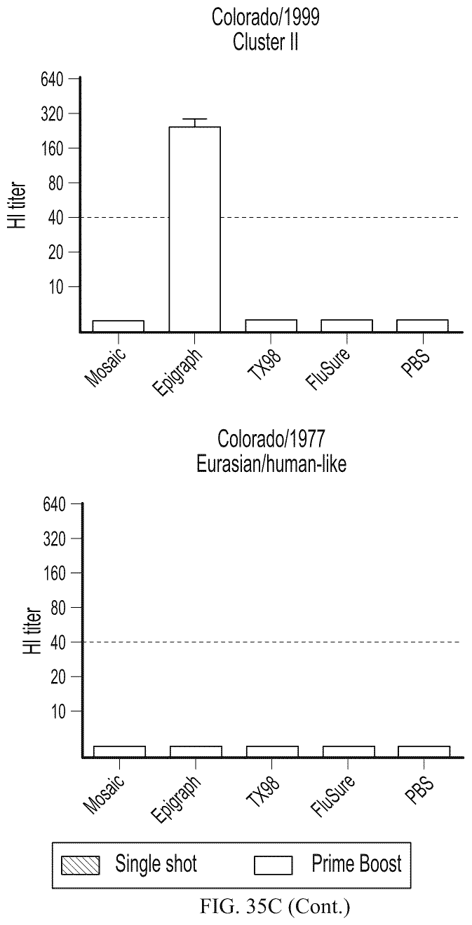

Groups of 5 to 10 mice were immunized with 1010 viral particles (vp) of Ad5-vectored vaccines expressing the Mosaic, Epigraph, or wild type TX98 HA immunogens (FIG. 35A). Additionally, mice were immunized with commercial FluSure at a dose equivalent to a 10× dose as compared to a 3-week old piglet. A submandibular bleed was performed at week 3 for evaluation of antibodies after a single immunization. Mice were then boosted and sera was harvested 14 days post-boost (FIG. 35A).

Representative divergent H3 swine influenza viruses were chosen for analysis of antibody development (FIG. 35B), and hemmaglutination inhibition (HI) were performed as described by the World Health Organization standard protocols (FIG. 35C). The dotted line represents the standard titer or 40 considered to confer protection against challenge.

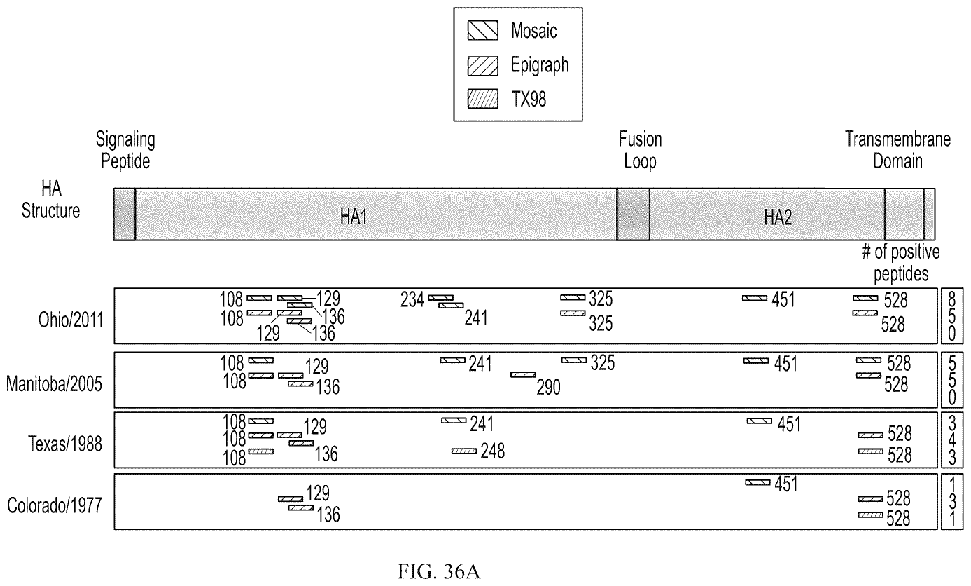

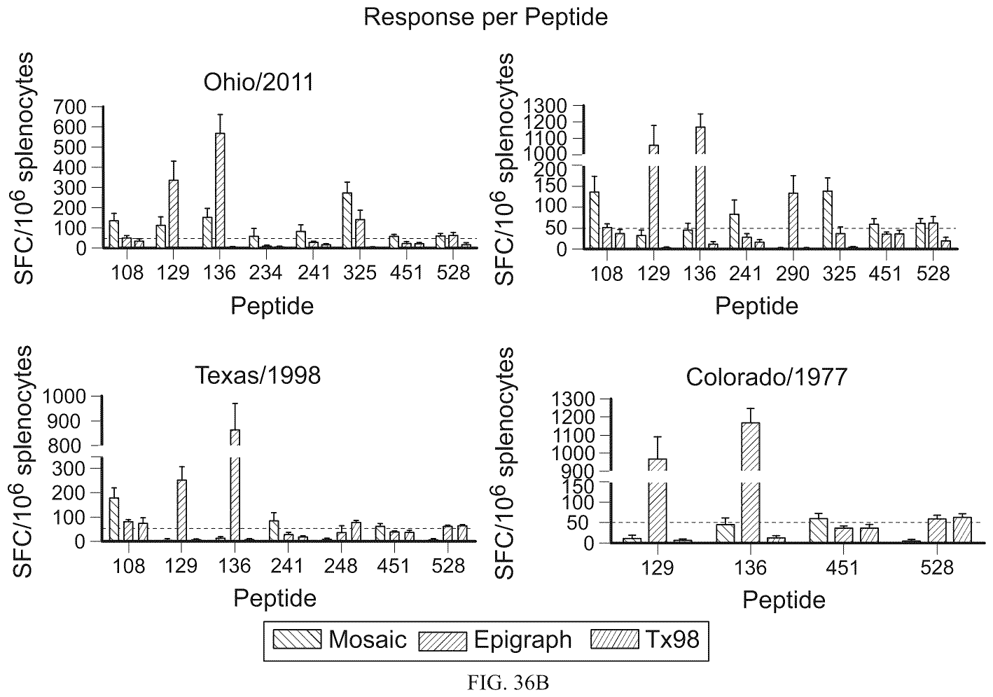

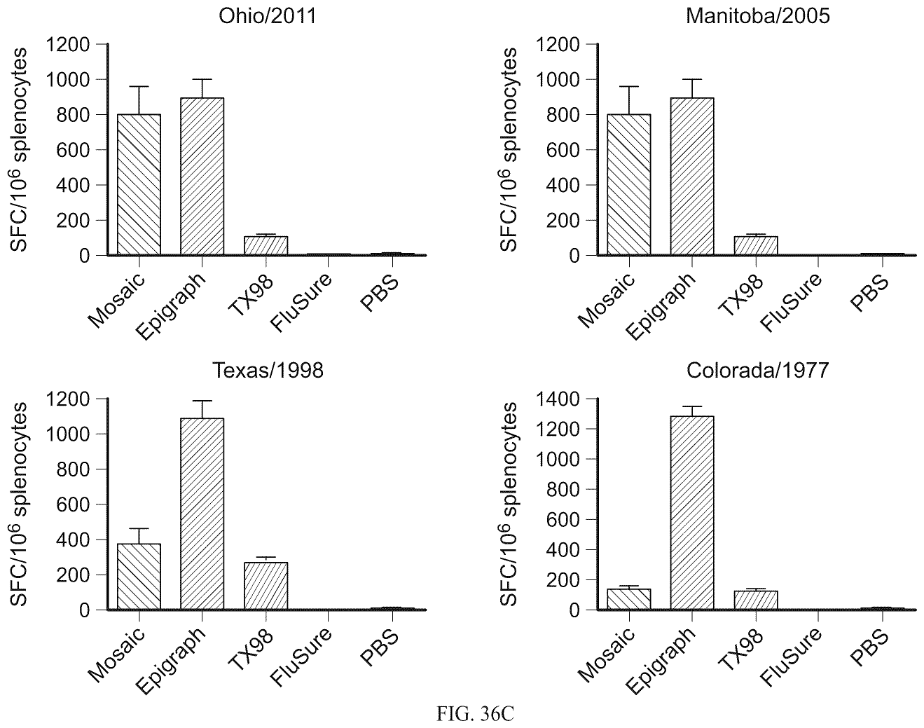

Balb/c mice (n=5) were vaccinated with Ad vectored vaccines expressing the Mosaic, Epigraph, and wild type TX98 HA immunogens as previously described in FIG. 35. Overlapping peptide libraries of 17-mers overlapping by 10 amino acids were used to determine the T cell epitopes induced by the vaccine immunogens to four representative divergent swH3 viruses. Interferon-γ inducing T cells were measured by ELISpot assay. A peptide was considered positive if the spot-forming cells (SFC) per million was greater than 50 (FIG. 36A). Individual responses to positive peptides (FIG. 36B) and total T-cell responses (FIG. 36C) were also measured.

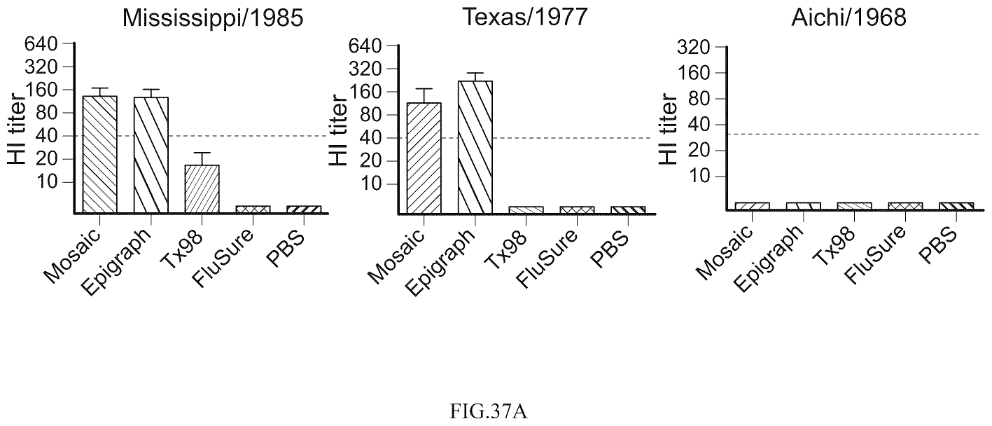

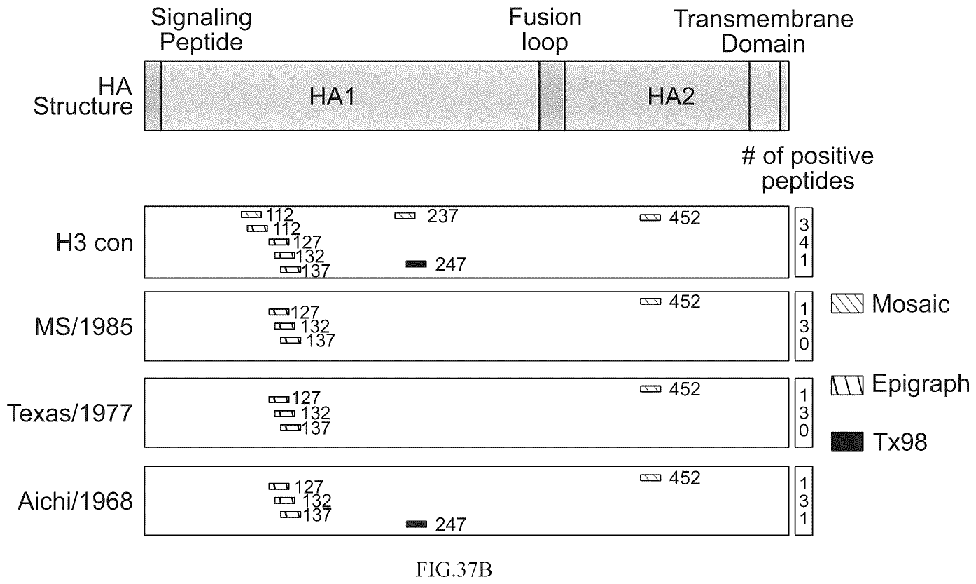

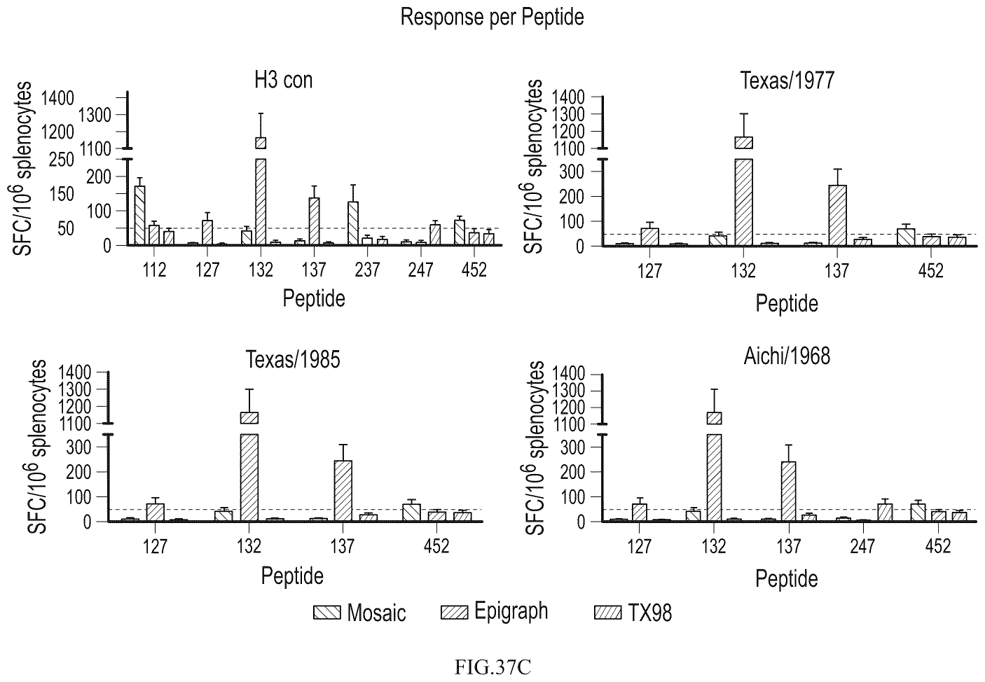

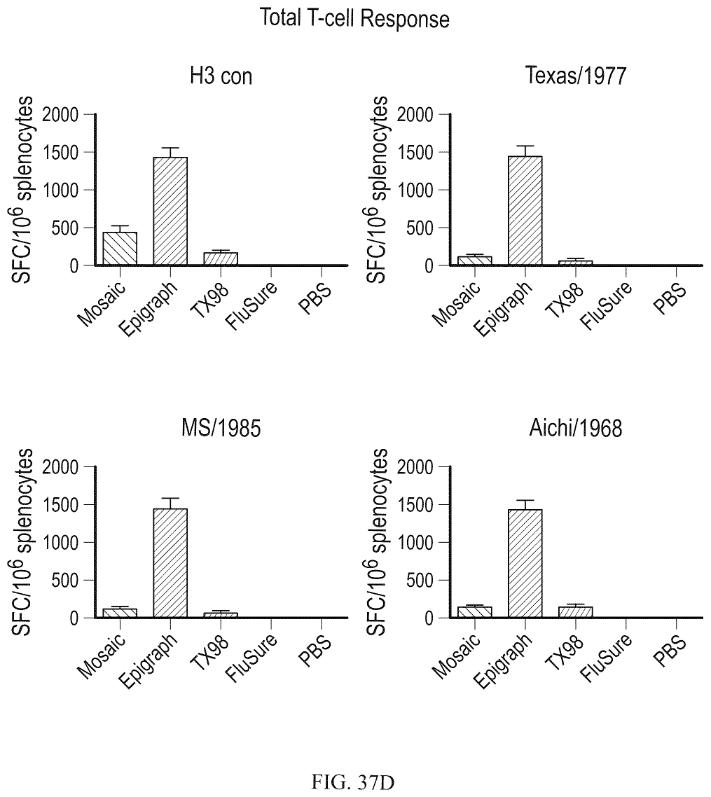

Balb/c mice (n=5) were immunized as previously described for FIG. 35. Sera from vaccinated mice was incubated with representative human H3N2 virus isolates and the geometric mean HI titer was determined±standard error (FIG. 37A). Splenocytes were evaluated for cross-reactive T-cell immune responses against four representative human influenza strains using overlapping peptide libraries and an IFN-γ ELISpot assay. A peptide was considered positive if the spot-forming cells (SFC) per million was greater than 50 (FIG. 37B). Individual responses to positive peptides (FIG. 37C) and total T-cell responses (FIG. 37D) were also measured.

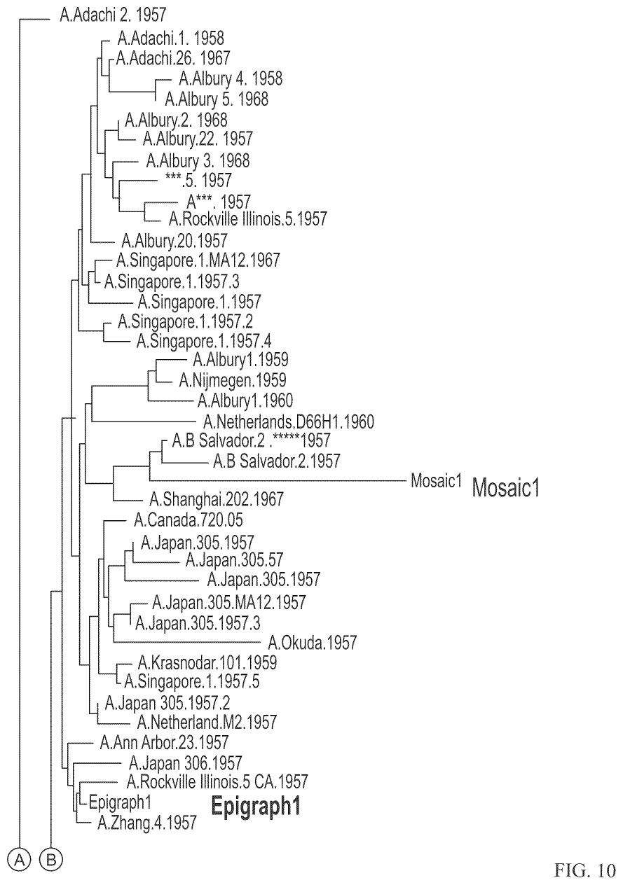

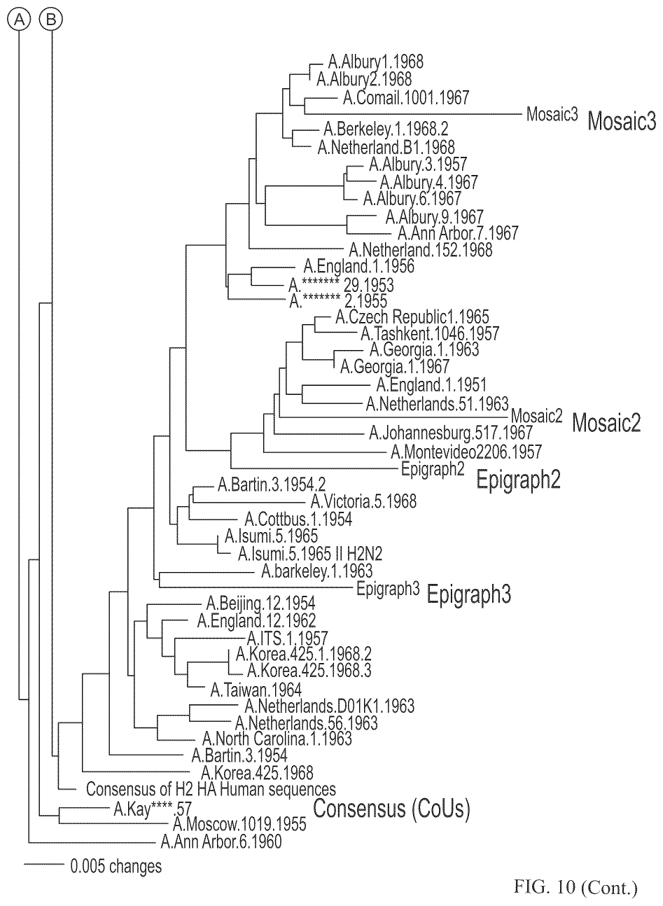

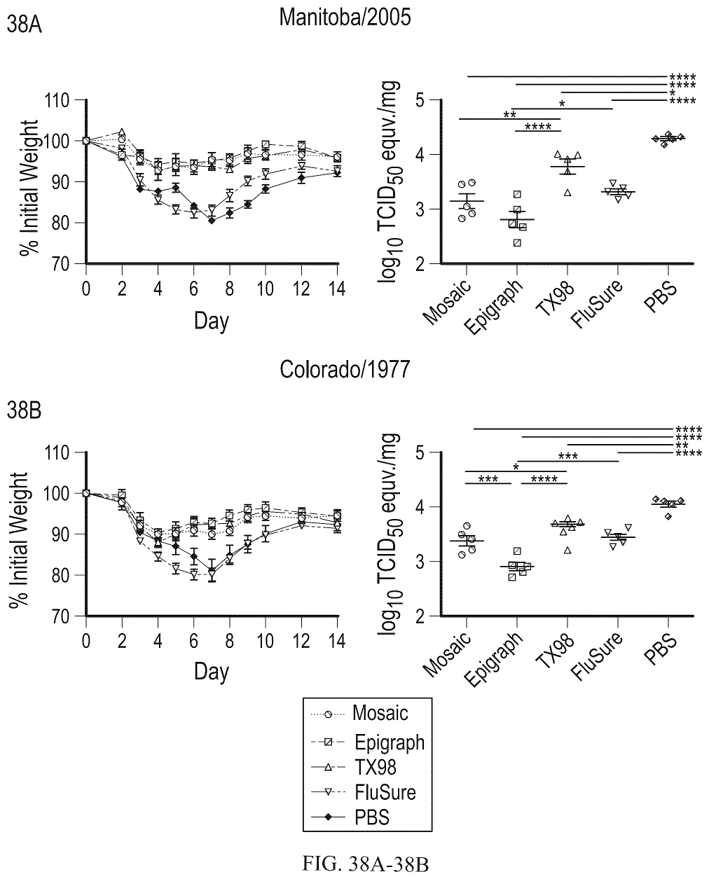

Balb/c mice (n=5) were vaccinated with a single dose of 10e10 vp of Ad5 vectored vaccines expressing the Mosaic, Epigraph, or TX98 immunogens and compared to FluSure vaccinated animals or sham vaccinated animals. Three weeks later mice were infected intranasally with 10e5 TCID50 of Manitoba/2005 (FIG. 38A) or 10e3.5 TCID50 of Colorado/1977 (FIG. 38B) and monitored for weight loss. In addition, group of mice (n=5) was vaccinated and infected as previously described but sacrificed on day 3 post infection for analysis of lung viral titer by qPCR (on the right).

It is to be understood that, while the methods and compositions of matter have been described herein in conjunction with a number of different aspects, the foregoing description of the various aspects is intended to illustrate and not limit the scope of the methods and compositions of matter. Other aspects, advantages, and modifications are within the scope of the following claims.

Disclosed are methods and compositions that can be used for, can be used in conjunction with, can be used in preparation for, or are products of the disclosed methods and compositions. These and other materials are disclosed herein, and it is understood that combinations, subsets, interactions, groups, etc. of these methods and compositions are disclosed. That is, while specific reference to each various individual and collective combinations and permutations of these compositions and methods may not be explicitly disclosed, each is specifically contemplated and described herein. For example, if a particular composition of matter or a particular method is disclosed and discussed and a number of compositions or methods are discussed, each and every combination and permutation of the compositions and the methods are specifically contemplated unless specifically indicated to the contrary. Likewise, any subset or combination of these is also specifically contemplated and disclosed.

What is claimed is:

1. A vaccine polypeptide having at least 90% sequence identity to an amino acid sequence selected from any of SEQ ID NOs: 6, 20, 34, 41, or 42.

2. The vaccine polypeptide of claim 1, wherein the polypeptide has at least 95% sequence identity to an amino acid sequence selected from any of SEQ ID NOs: 6, 20, 34, 41, or 42.

3. The vaccine polypeptide of claim 1, wherein the polypeptide has at least 99% sequence identity to an amino acid sequence selected from any of SEQ ID NOs: 6, 20, 34, 41, or 42.

4. The vaccine polypeptide of claim 1, wherein the polypeptide has 100% sequence identity to an amino acid sequence selected from any of SEQ ID NOs: 6, 20, 34, 41, or 42.

5. The vaccine polypeptide of claim 1, wherein the polypeptide having SEQ ID NO:6 or 20 is encoded by a nucleic acid sequence having at least 90% sequence identity to a nucleic acid sequence shown in SEQ ID NOs: 105 or 110, respectively.

6. The vaccine polypeptide of claim 1, wherein the polypeptide having SEQ ID NO:6 or 20 is encoded by a nucleic acid sequence having at least 95% sequence identity to a nucleic acid sequence shown in SEQ ID NOs: 105 or 110, respectively.

7. The vaccine polypeptide of claim 1, wherein the polypeptide having SEQ ID NO:6 or 20 is encoded by a nucleic acid sequence having at least 99% sequence identity to a nucleic acid sequence shown in SEQ ID NOs: 105 or 110, respectively.

8. The vaccine polypeptide of claim 1, wherein the polypeptide having SEQ ID NO:6 or 20 is encoded by a nucleic acid sequence having at least 100% sequence identity to a nucleic acid sequence shown in SEQ ID NOs: 105 or 110, respectively.

9. A vaccine composition comprising at least one of the vaccine polypeptides of claim 1 and a delivery vehicle.

10. The vaccine composition of claim 9, wherein the delivery vehicle is a virus.

11. The vaccine composition of claim 10, wherein the delivery vehicle virus is selected from the group consisting of an adenovirus, an adeno-associated virus, a retrovirus, an alphavirus, a paramyxovirus, and a rhabdovirus.

12. The vaccine composition of claim 9, wherein the delivery vehicle is a nanoparticle.

13. A method of vaccinating a subject, the method comprising:
administering the vaccine polypeptide of claim 1 to a subject in need of vaccination.

14. The method of claim 13, wherein the subject is selected from the group consisting of a human and a swine.

15. The method of claim 13, wherein the administering step is repeated more than once.
